(19) United States
(12) Patent Application Publication
(10) Pub. No.: US 2009/0192194 A1
(43) Pub. Date: Jul. 30, 2009

(54) Cyclic Amine Derivatives, Processes For Their Preparation, And Pharmaceutical Compositions Containing Them

(76) Inventors: Giuseppe Alvaro, Verona (IT); Luca Arista, Verona (IT); Francesca Cardullo, Verona (IT); Lucilla D'Adamo, Verona (IT); Aldo Feriani, Verona (IT); Riccardo Giovannini, Verona (IT); Catia Seri, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/869,945

(22) Filed: Oct. 10, 2007

Related U.S. Application Data
(63) Continuation of application No. 10/554,822, filed on Jun. 1, 2006.

(30) Foreign Application Priority Data
May 9, 2003 (GB) 0310724.0

(51) Int. Cl. A61K 31/445; C07D 401/02; C07D 211/22; A61P 25/22

(52) U.S. Cl. 514/321; 546/196; 546/197; 514/320

(57) ABSTRACT

A compound of formula (I)

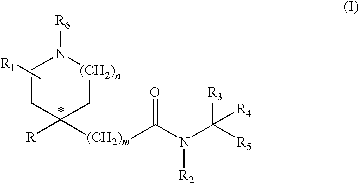

wherein R is a radical selected from

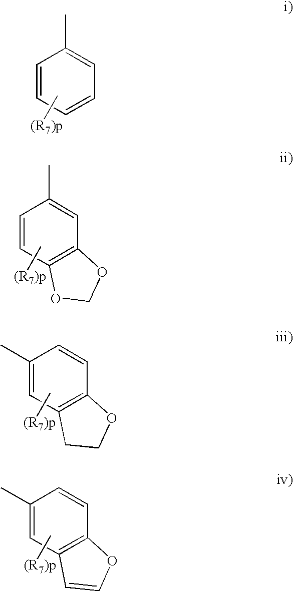

in which R₇ is halogen, cyano, C₁₋₄ alkyl, C₁₋₄ alkoxy, trifluoromethyl or trifluoromethoxy;
p is an integer from 0 to 3;
R₁ is hydrogen, halogen, cyano, C₂₋₄ alkenyl, C₁₋₄ alkyl optionally substituted by halogen, cyano or C₁₋₄ alkoxy;
R₂ is hydrogen or C₁₋₄ alkyl;
R₃ and R₄ independently are hydrogen, C₁₋₄ alkyl or R₃ together with R₄ is C₃₋₇ cycloalkyl;
R₅ is phenyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl, naphthyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl, a 9 to 10 membered fused bicyclic heterocyclic group substituted by 1 to 3 groups independently selected from trifluoromethyl, C₁₋₄ alkyl, cyano, C₁₋₄ alkoxy, trifluoromethoxy, halogen or (SO)rC₁₋₄ alkyl or R₅ is a 5 or 6 membered heteroaryl group substituted by 1 to 3 groups independently selected from trifluoromethyl, C₁₋₄ alkyl, cyano, C₁₋₄ alkoxy, trifluoromethoxy, halogen or (SO)rC₁₋₄ alkyl;
R₆ is hydrogen or (CH₂)qR₈;
R₈ is hydrogen, C₃₋₇ cycloalkyl, C₁₋₄ alkoxy, amine, C₁₋₄ alkylamine, (C₁₋₄ alkyl)₂amine, OC(O)NR₉R₁₀ or C(O)NR₉R₁₀;
R₉ and R₁₀ independently are hydrogen, C₁₋₄ alkyl or C₃₋₇ cycloalkyl;
m is zero or 1;
n is 1 or 2;
q is an integer from 1 to 4;
r is 1 or 2;
   or pharmaceutically acceptable salts and solvates thereof; processes for their preparation to pharmaceutical compositions containing them and their use in the treatment of conditions mediated by tachykinins and/or by selective inhibition of serotonin reuptake transporter protein.

The present invention relates to cyclic amine derivatives, to processes for their preparation, to pharmaceutical compositions containing them and to their medical use.

WO 20044005256 discloses certain cyclic amine derivatives as tachykinins receptors (especially Nk1 receptor) antagonists and as selective serotonin reuptake inhibitors (SSRIs). Such compounds are useful for the treatment of CNS disorders and psychotic disorders, in particular in the treatment or prevention of depressive states and/or in the treatment of anxiety.

However, in the above cited document there is neither disclosure nor suggestion of any compound as claimed herein.

Thus, the present invention provides compounds of formula (I)

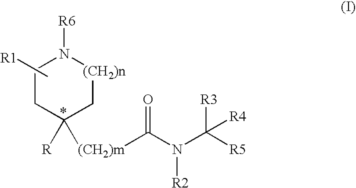

wherein R represents a radical selected from

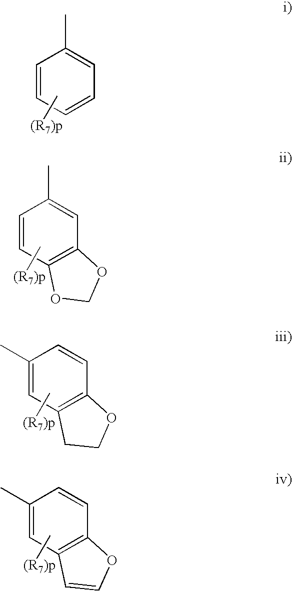

in which R₇ is halogen, cyano, C₁₋₄ alkyl, C₁₋₄ alkoxy, trifluoromethyl or trifluoromethoxy;
p is an integer from 0 to 3;
R₁ represents hydrogen, halogen, cyano, C₂₋₄ alkenyl, C₁₋₄ alkyl optionally substituted by halogen, cyano or C₁₋₄ alkoxy;
R₂ represents hydrogen or C₁₋₄ alkyl;
R₃ and R₄ independently represent hydrogen, C₁₋₄ alkyl or R₃ together with R₄ represent C₃₋₇ cycloalkyl;
R₅ represents:
phenyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl,
naphthyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl,
a 9 to 10 membered fused bicyclic heterocyclic group substituted by 1 to 3 groups independently selected from trifluoromethyl, C₁₋₄ alkyl, cyano, C₁₋₄ alkoxy, trifluoromethoxy, halogen or (SO)rC₁₋₄ alkyl or
R₅ is a 5 or 6 membered heteroaryl group substituted by 1 to 3 groups independently selected from trifluoromethyl, C₁₋₄ alkyl, cyano, C₁₋₄ alkoxy, trifluoromethoxy, halogen or (SO)rC₁₋₄ alkyl;
   R₆ represents hydrogen or (CH₂)qR₈;
   R₈ represents hydrogen, C₃₋₇ cycloalkyl, C₁₋₄ alkoxy, amine, C₁₋₄ alkylamine, (C₁₋₄ alkyl)₂amine, OC(O)NR₉R₁₀ or C(O)NR₉R₁₀;
   R₉ and R₁₀ independently represent hydrogen, C₁₋₄ alkyl or C₃₋₇ cycloalkyl;
   m represents zero or 1;
   n is 1 or 2;
   q is an integer from 1 to 4;
   r is 1 or 2;
   provided that when R₅ is phenyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl, R is not the radical i)

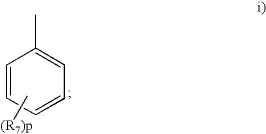

or pharmaceutically acceptable salts or solvates thereof.

A further embodiment of the invention provides compounds of formula (I) or pharmaceutically acceptable salts and solvates thereof wherein R represents a radical selected from

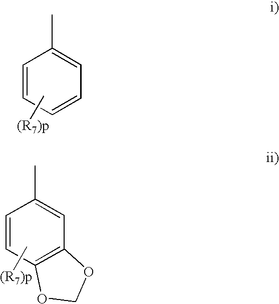

in which R₇ is halogen, cyano, C₁₋₄ alkyl, C₁₋₄ alkoxy, trifluoromethyl or trifluoromethoxy;
p is an integer from 0 to 3;
R₁ represents hydrogen, halogen, cyano, C₁₋₄ alkyl optionally substituted by halogen, cyano, C₁₋₄ alkoxy;
R₂ represents hydrogen or Cₗ₋₄ alkyl;
R₃ and R₄ independently represent hydrogen, C₁₋₄ alkyl or R₃ together with R₄ represent C₃₋₇ cycloalkyl;
R₅ represents substituted phenyl, substituted naphthyl, a substituted 9 to 10 membered fused bicyclic heterocyclic group or a substituted 5 or 6 membered heteroaryl group, wherein said groups are substituted by 1 to 3 groups independently selected from trifluoromethyl, C₁₋₄ alkyl, cyano, C₁₋₄ alkoxy, trifluoromethoxy, halogen or (SO)rC₁₋₄ alkyl;
R₆ represents hydrogen or (CH₂)qR₈;
R₈ represents hydrogen, C₃₋₇ cycloalkyl, C₁₋₄ alkoxy, amine, C₁₋₄ alkylamine, (C₁₋₄ alkyl)₂-amine, OC(O)NR₉R₁₀ or C(O)NR₉R₁₀;
R₉ and R₁₀ independently represent hydrogen, C₁₋₄ alkyl or C₃₋₇ cycloalkyl;
m represents zero or an integer from 1 to 4;
n is 1 or 2;
q is an integer from 1 to 4;
r is 1 or 2;
provided that when R₅ is phenyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl, R is not the radical i)

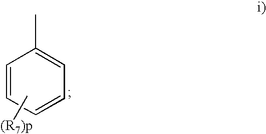

Suitable pharmaceutically acceptable salts of the compounds of general formula (I) include acid addition salts formed with pharmaceutically acceptable organic or inorganic acids, for example hydrochlorides, hydrobromides, sulphates, alkyl- or arylsulphonates (e.g. methanesulphonates or p-toluenesulphonates), phosphates, trifluoroacetates, acetates, citrates, succinates, tartrates, lactates, malates, fumarates and maleates.

The solvates may, for example, be hydrates.

References hereinafter to a compound according to the invention include both compounds of formula (I) and their pharmaceutically acceptable acid addition salts and their pharmaceutically acceptable solvates.

It will be appreciated by those skilled in the art that the compounds of formula (I), when n is 1 and R₁ is not hydrogen or when n is 2, contain at least one asymmetric carbon atom (namely the carbon atom shown as * in formula (I)) and may be represented by formula (1a) and (1b).

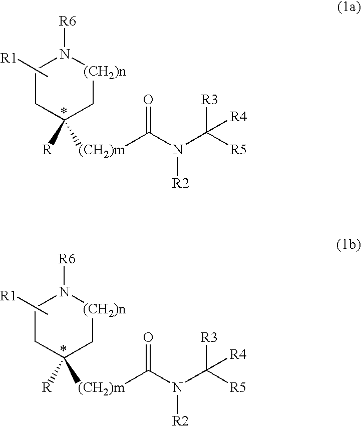

The wedge bond indicates that the bond is above the plane of the paper. The broken bond indicates that the bond is below the plane of the paper.

At least two asymmetric carbon atoms are present in the compounds of formula (I) when R₁ is different from hydrogen (namely the carbon atom shown as * in formula (I) and the carbon atom to which the group R₁ is attached) and may be represented by formula (1a) (1b), (1c) and (1d).

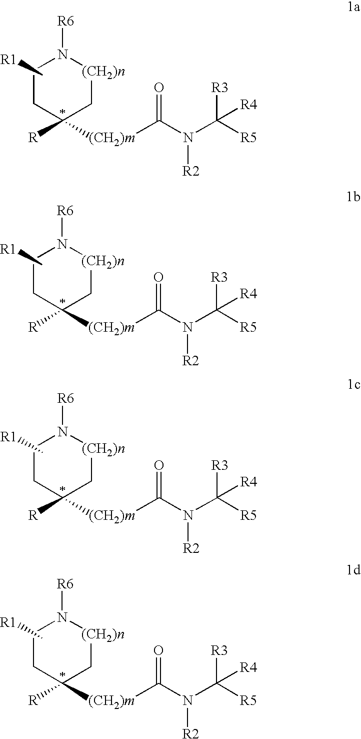

For compounds of the invention wherein m is 1, the configuration of the asymmetric carbon atoms of the compounds shown in formulae 1a and 1d is hereinafter referred to as syn isomer and in formulae 1b and 1c as the anti isomer.

For compounds of the invention wherein m is 0, the configuration of the asymmetric carbon atoms of the compounds shown in formulae 1b and 1c is hereinafter referred to as syn isomer and in formulae 1a and 1d as the anti isomer.

Further asymmetric carbon atoms are possible when R₃ and R₄ are not the same group namely the carbon atom identified as **in the formula (Ia)

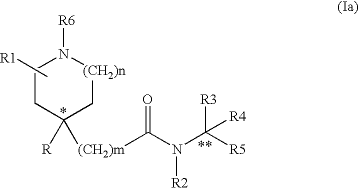

Thus, for example, when R₁ is a group different from hydrogen and R₃ and R₄ are not the same group, at least three asymmetric carbon atoms are present in the compounds of formula (I) and may be represented by formula (1e) (1f), (1 g), (1 h), 1(i), (1l), (1m) and (1n)

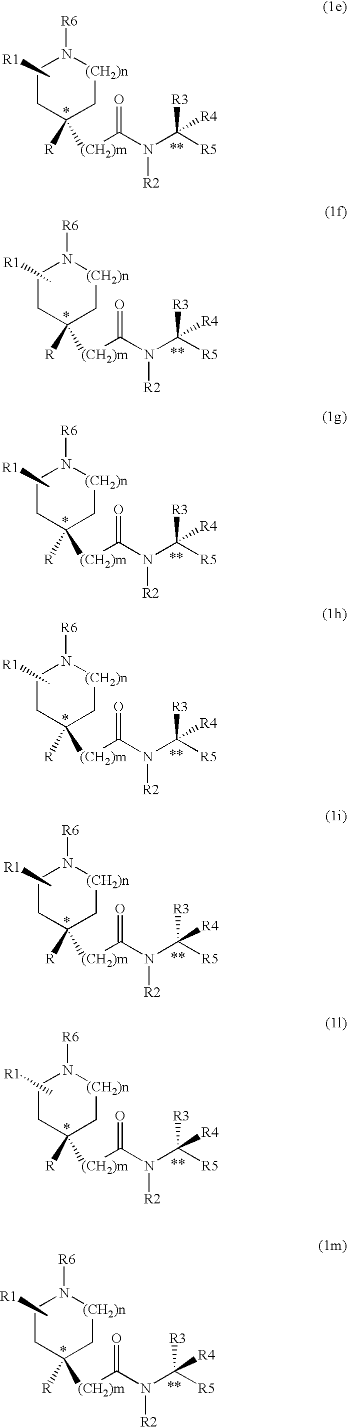

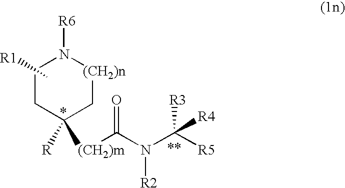

It is to be understood that all stereoisomeric forms including all enantiomers and diastereoisomers and mixtures thereof are encompassed within the scope of the present invention and the reference to compounds of formula (I) include all stereisomeric forms unless otherwise stated.

Furthermore, the compounds of formula (I) may exist in one or more crystalline forms and the crystalline forms of the compounds of structure (I) may exist as polymorphs, which are included in the present invention.

The present invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I and following of this invention can generally be prepared by carrying out the procedures disclosed in the Schemes and/or in the Examples below, by substituting a readily available isotopically labeled reagent for a non-isotopically labeled reagent.

The term C₁₋₄ alkyl as used herein as a group or a part of the group refers to a straight or branched alkyl group containing from 1 to 4 carbon atoms; examples of such groups include methyl, ethyl, propyl, isopropyl, n-butyl, isobutyl, tert-butyl.

The term C₂₋₄ alkenyl refers to a straight or branched alkylene group containing from 2 to 4 carbon atoms; examples of such groups include ethenyl, 1-propenyl, allyl, butenyl and the like.

The term halogen refers to fluorine, chlorine, bromine or iodine.

The term C₃₋₇ cycloalkyl group means a non aromatic monocyclic hydrocarbon ring of 3 to 7 carbon atoms such as, for example, cyclopropyl, cyclobutyl, cyclopentyl, cyclohexyl or cycloheptyl.

The term C₁₋₄ alkoxy group may be a straight chain or a branched chain alkoxy group, for example methoxy, ethoxy, propoxy, prop-2-oxy, butoxy, but-2-oxy or methylprop-2-oxy.

When R₅ is a 5 or 6 membered heteroaryl group according to the invention this includes furanyl, thiophenyl, pyrrolyl, imidazolyl, thiazolyl, oxazolyl, pyrazolyl, isoxazolyl, isothiazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl or 1,3,5-triazinyl and the like.

The term 9 to 10 membered fused bicyclic heterocyclic group refers to a 5,6/6, 5 or 6,6 bicyclic ring system, containing at least one heteroatom selected from oxygen, sulphur or nitrogen, which may be saturated, unsaturated or aromatic. The term 9 to 10 membered fused bicyclic heterocyclic group also refers to a phenyl fused to one 5 or 6 membered heterocyclic group. Example of such groups include benzofuranyl, benzothiophenyl, indolyl, benzoxazolyl, 3H-imidazo[4,5-c]pyridin-yl, dihydrophthazinyl, 1H-imidazo[4,5-c]pyridin-1-yl, imidazo[4,5-b]pyridyl, 1,3-benzo[1,3]dioxolyl, 2H-chromanyl, isochromanyl, 5-oxo-2,3-dihydro-5H-[1,3]thiazolo[3,2-a]pyrimidyl, 1,3-benzothiazolyl, 1,4,5,6-tetrahydropyridaziyl, 1,2,3,4,7,8-hexahydropteridinyl, 2-thioxo-2,3,6,9-tetrahydro-1H-purin-8-yl, 3,7-dihydro-1H-purin-8-yl, 3,4-dihydropyrimidin-1-yl, 2,3-dihydro-1,4-benzodioxinyl, benzo[1,3]dioxolyl, 2H-chromenyl, chromanyl, 3,4-dihydrophthalazinyl, 2,3-dihydro-1H-indolyl, 1,3-dihydro-2H-isoindol-2-yl, 2,4,7-trioxo-1,2,3,4,7,8-hexahydropteridinyl, thieno[3,2-d]pyrimidinyl, 4-oxo-4,7-dihydro-3H-pyrrolo[2,3-d]pyrimidinyl, 1,3-dimethyl-6-oxo-2-thioxo-2,3,6,9-tetrahydro-1H-purinyl, 1,2-dihydroisoquinolinyl, 2-oxo-1,3-benzoxazolyl, 2,3-dihydro-5H-1,3-thiazolo[3,2-a]pyrimidinyl, 5,6,7,8-tetrahydro-quinazolinyl, 4-oxochromanyl, 1,3-benzothiazolyl, benzimidazolyl, benzotriazolyl, purinyl, furylpyridyl, thiophenylpyrimidyl, thiophenylpyridyl, pyrrolylpiridyl, oxazolylpyridyl, thiazolylpiridyl, 3,4-dihydropyrimidin-1-yl imidazolylpiridyl, quinoliyl, isoquinolinyl, quinazolinyl, quinoxalinyl, naphthyridinyl, pyrazolyl[3.4]pyridine, 1,2-dihydroisoquinolinyl, cinnolinyl, 2,3-dihydro-benzo[1,4]dioxin-6-yl, 4,5,6,7-tetrahydro-benzo[b]thiophenyl-2-yl, 1,8-naphthyridinyl, 1,6-naphthyridinyl, 3,4-dihydro-2H-1,4-benzothiazine, 4,8-dihydroxy-quinolinyl, 1-oxo-1,2-dihydro-isoquinolinyl or 4-phenyl-[1,2,3]thiadiazolyl and the like.

In the compounds of formula (I) wherein n is 1 the group R₁ may be in position 2, 3, 5 or 6 of the piperidine ring as represented in formula (Ib). Wherein these compounds R₁ in the position 2 or 6 is preferred.

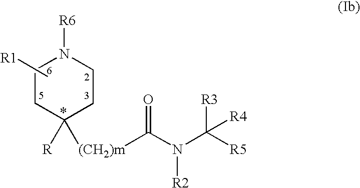

In the compounds of formula (I) wherein n is 2 the group R₁ may be in position 2, 3, 4, 6 or 7 of the ring as represented in formula (Ic)

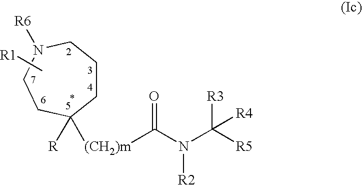

For compounds of formula (I) n is preferably 1.

For compounds of formula (I) m is preferably 1.

R is preferably phenyl in which R₇ is preferably halogen (e.g fluorine or chlorine), cyano, trifluoromethyl, C₁₋₄ alkyloxy(e.g methoxy), or C₁₋₄ alkyl (e.g methyl) and within this class p is preferably 0 or an integer from 1 to 2 or R is preferably a group selected from

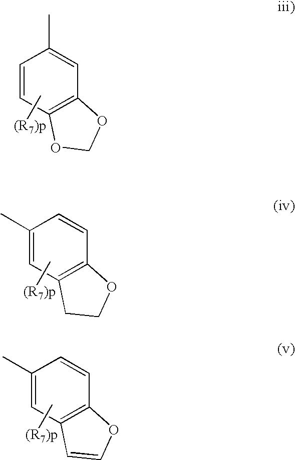

wherein p is 0.

R₁ is preferably hydrogen, C₂₋₄ alkenyl(e.g ethenyl), halogen (e.g fluorine) or C₁₋₄ alkyl (e.g methyl). Within this class those compounds wherein R₁ is in the 1 or 2 position of the piperidine ring are particularly preferred.

R₂ is preferably hydrogen or methyl.

R₃ is preferably hydrogen or methyl.

R₄ is preferably hydrogen or methyl.

When R₅ is substituted phenyl, this is preferably substituted by one or 2 groups selected from halogen (e.g fluorine, bromine or chlorine), cyano, trifluoromethyl or C₁₋₄ alkyl (e.g methyl)

When R₅ is substituted naphthyl, this is preferably a 1-naphthyl group substituted by one or 2 groups selected from halogen (e.g fluorine, bromine or chlorine), cyano, trifluoromethyl or C₁₋₄ alkyl (e.g methyl).

When R₅ is a substituted 9 to 10 membered fused bicyclic heterocyclic group this is preferably benzofuranyl (e.g-benzofuran-7-yl or benzofuran-4-yl), benzothiophenyl (e.g benzothiophen-4-yl or benzothiophen-7-yl) indolyl (indol-4-yl or indol-7-yl) or benzoxazolyl, wherein said groups are substituted by one group selected from halogen (e.g fluorine, bromine or chlorine), cyano, trifluoromethyl or C₁₋₄ alkyl (e.g methyl).

When R₅ is a substituted 5 or 6 membered heteroaryl group this is preferably furanyl (e.g furan-2-yl or furan-3-yl), thiophenyl or pyrrolyl, wherein said groups are substituted by one group selected from halogen (e.g fluorine, bromine or chlorine), cyano, trifluoromethyl or C₁₋₄ alkyl (e.g methyl).

R₆ is preferably hydrogen or C₁₋₄ alkyl (e.g methyl).

R₅ is more preferably phenyl substituted by one or two groups selected from fluorine, bromine, chlorine, cyano, or methyl, naphthyl substituted by one or two groups selected from fluorine, bromine, chlorine, cyano, or methyl, benzofuranyl substituted by one or two groups selected from fluorine, bromine, chlorine, cyano, or methyl, or R₅ is furanyl substituted by one or two groups selected from fluorine, bromine, chlorine, cyano, or methyl.

A preferred class of compounds of formula (I) includes those wherein n and m is 1.

A further preferred class of compounds is that wherein R₂, R₃ and R₄ are independently hydrogen or methyl.

A further preferred class of compounds is that wherein n is 1, m is 1, R₂, R₃ and R₄ are independently hydrogen or methyl and R₆ is hydrogen or C₁₋₄ alkyl.

A preferred group of compounds of formula (I) includes those wherein n is 1, m is 1, R₂ is hydrogen or methyl, R₃ is hydrogen, R₄ is hydrogen or methyl, R₆ is hydrogen or methyl and R₁ is hydrogen, C₂₋₄ alkenyl, halogen or C₁₋₄ alkyl at the 1 or 2 position of the piperidine ring.

A further preferred group of compounds of formula (I) includes those wherein n and m are 1, R₂ is hydrogen or methyl, R₃ is hydrogen, R₄ is hydrogen or methyl, R₅ is phenyl (substituted by one or two groups selected from fluorine, bromine, chlorine, cyano, or methyl), naphthyl (substituted by one or two groups selected from fluorine, bromine, chlorine, cyano, or methyl), benzofuranyl (substituted by one or two groups selected from fluorine, bromine, chlorine, cyano, or methyl), or R₅ is furanyl (substituted by one or two groups selected from fluorine, bromine, chlorine, cyano, or methyl), or R₅ is benzofuranyl (substituted by a fluorine, bromine, chlorine, cyano, or methyl), R₆ is hydrogen or methyl and R₁ is hydrogen, C₂₋₄ alkenyl, C₁₋₄ alkyl or halogen at the 1 or 2 position of the piperidine ring.

A further preferred group of compounds of formula (I) includes those wherein n and m are 1, R₂ is hydrogen or methyl, R₃ is hydrogen, R₄ is hydrogen or methyl, R₅ is phenyl (substituted by one or two groups selected from fluorine, bromine, chlorine, cyano, or methyl), naphthyl (substituted by one or two groups selected from fluorine, bromine, chlorine, cyano, or methyl), benzofuranyl (substituted by one or two groups selected from fluorine, bromine, chlorine, cyano, or methyl), or R₅ is furanyl (substituted by one or two groups selected from fluorine, bromine, chlorine, cyano, or methyl), or R₅ is benzofuranyl (substituted by a fluorine, bromine, chlorine, cyano, or methyl), R₆ is hydrogen or methyl, R₁ is hydrogen, C₂₋₄ alkenyl, C₁₋₄ alkyl or halogen at the 1 or 2 position of the piperidine ring and R is phenyl in which R₇ is halogen, trifluoromethyl, cyano, C₁₋₄ alkoxy or C₁₋₄ alkyl and p is 0 or an integer from 1 to 2 or R is a group selected from

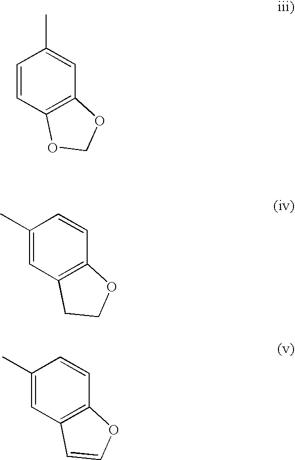

wherein p is 0.

A further preferred group of compounds of formula (I) includes those wherein n and m are 1, R₂ hydrogen or methyl, R₃ is hydrogen, R₄ is hydrogen or methyl, R₅ is phenyl (substituted by one or two groups selected from fluorine, bromine, chlorine, cyano, or methyl), naphthyl (substituted by one or two groups selected from fluorine, bromine, chlorine, cyano, or methyl), benzofuranyl (substituted by one or two groups selected from fluorine, bromine, chlorine, cyano, or methyl) or R₅ is benzofuranyl (substituted by a fluorine, bromine, chlorine, cyano, or methyl), R₆ is hydrogen or methyl, R₁ is hydrogen C₂₋₄ alkenyl, C₁₋₄ alkyl or halogen at the 1 or 2 position of the piperidine ring and R is phenyl in which R₇ is halogen, trifluoromethyl, cyano, C₁₋₄ alkoxy or C₁₋₄ alkyl and p is 0 or an integer from 1 to 2 or R is a group selected from

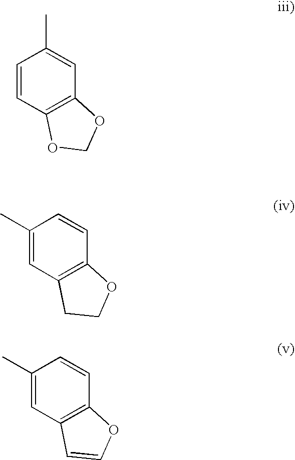

wherein p is 0.

Another further preferred group of compounds of formula (I) includes those wherein n and m are 1, R₂ is hydrogen or methyl, R₃ is hydrogen, R₄ is hydrogen or methyl, R₅ is phenyl substituted by one or two groups selected from fluorine, bromine or chlorine, cyano, or methyl, 1-naphthyl substituted by one or two groups selected from fluorine, bromine or chlorine, cyano, or methyl, or R₅ is benzofuran-7-yl substituted by a fluorine, bromine or chlorine, cyano, or methyl, R₆ is hydrogen or methyl, R₁ is hydrogen or ethenyl, fluorine or methyl at the 1 or 2 position of the piperidine ring and R is phenyl in which R₇ is fluorine, methoxy, cyano or methyl and p is 0 or an integer from 1 to 2 or R is a group selected from

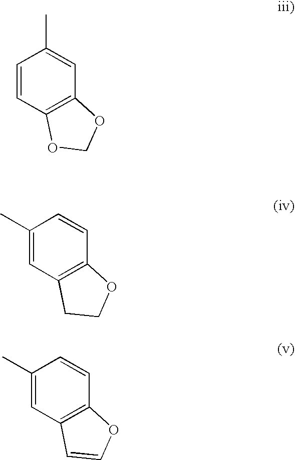

wherein p is 0.

Specific preferred compounds according to the invention are:
N-[1-(3-chloro-1-naphthalenyl)ethyl]-2-[4-(4-fluorophenyl)-1-methyl-4-piperidinyl]-N-methylacetamide (Enantiomer 1);
N-[1-(3-chloro-1-naphthalenyl)ethyl]-N-methyl-2-(1-methyl-4-phenyl-4-piperidinyl)acetamide (Enantiomer 1);
N-[1-(3-chloro-1-naphthalenyl)ethyl]-N-methyl-2-(1-methyl-4-phenyl-4-piperidinyl)acetamide (Enantiomer 2);
2-[4-(1-benzofuran-5-yl)-1-methyl-4-piperidinyl]-N-[1-(3-chloro-1-naphthalenyl)ethyl]-N-methylacetamide (Enantiomer 1);
N-[1-(3-chloro-1-naphthalenyl)ethyl]-N-methyl-2-{1-methyl-4-[4-(methyloxy)phenyl]-4-piperidinyl}acetamide (Enantiomer 1);
N-[1-(3-chloro-1-naphthalenyl)ethyl]-2-[4-(4-fluorophenyl)-1,2-dimethyl-4-piperidinyl]-N-methylacetamide (Syn isomer 2, chain enantiomer 1);
N-[1-(3-chloro-1-naphthalenyl)ethyl]-2-(1,2-dimethyl-4-phenyl-4-piperidinyl)-N-methylacetamide (Syn isomer 2, chain enantiomer 1);
   or pharmaceutically acceptable salts or solvates thereof.

It will be appreciated that the chemical compounds can be named in different ways and according to different naming conventions.

The compounds of the invention are antagonists of tachykinin receptors, including substance P and other neurokinins, both in vitro and in vivo and are thus of use in the treatment of conditions mediated by tachykinins, including substance P and other neurokinins.

Tachykinins are a family of peptides that share a common carboxyl-terminal sequence (Phe-X-Gly-Leu-Met-NH2). They are actively involved in the physiology of both lower and advanced lifeforms. In mammalian lifeforms the main tachykinins are substance P (SP), Neurokinin A (NKA) and Neurokinin B (NKB) which act as neurotransmitters and neuromodulators. Mammalian tachykinins may contribute to the pathophysiology of a number of human diseases.

Three types of tachykinins receptors have been identified, namely NK1 (SP-preferring), NK2 (NKA-preferring) and NK3 (NKB-preferring) which are widely distributed throughout the central nervous (CNS) and peripheral nervous system.

Particularly the compounds of the invention are antagonists of the NK1 receptor.

The compounds of the present invention also have activity as selective serotonin reuptake inhibitors (hereinafter referred to as SSRIs) and are thus of use in the treatment of conditions mediated by selective inhibition of the serotonin reuptake transporter protein.

Thus, the compounds of the present invention combine dual activity as tachykinin antagonists, including substance P and other neurokinins, and as SSRIs. In particular, the compounds of the invention combine dual activity as NK₁ receptor antagonists and as SSRIs.

NK₁-receptor binding affinity has been determined in vitro in a binding Scintillation proximity assay (SPA) by measuring the compounds' ability to displace [¹²⁵I]Tyr8-Substance P (SP) from recombinant human NK₁ receptors stably expressed in Chinese Hamster Ovary (CHO) cell membranes prepared by using a modification of the method described by Beattie D. T. et al. (Br. J. Pharmacol, 116:3149-3157, 1995). Briefly, polystrene Leadseeker WGA-SPA beads (Amersham Biosciences) were mixed with cell membranes in a bead/membrane ratio of 50:1 (w/w) in assay buffer (75 mM Tris pH 7.8, 75 mM NaCl, 4 mM MnCl2, 1 mM EDTA, 0.05% Chaps, 1 mM PMSF). The mixture was placed on ice for 30 minutes to allow the formation of membrane/bead complex before BSA was added to a final concentration of 1%. After another 30 minutes incubation on ice, the bead/membrane complex was washed twice and suspended in assay buffer. [¹²⁵I]Tyr8-Substance P (2200 Ci/mmol, PerkinElmer) was then added to the bead/membrane complex with a final concentration of 0.4 nM. 30 ul of the resulting mixture was then dispensed to each well of Nalgen NUNC 384-well plate with 1 ul compound pre-dispensed in DMSO. The plates were then sealed and pulse centrifuged at 1100 rpm. After 3 hours incubation at room temperature with shaking, the plates were centrifuged for 2 min at 1100 rpm and measured in Viewlux imager (PerkinElmer) for 5 minutes with a 618-nm filter. Inhibition of [¹²⁵I]Tyr8-Substance P binding to NK₁-receptors was measured by the reduction of luminescent signal. IC₅₀ values of each compound were determined by an 11-point 3×-dilution inhibition curve. pKᵢ values were calculated using the K_{D} of [¹²⁵I]Tyr8-Substance P determined in a separate experiment.

For preferred compounds of the invention NK₁-receptor binding affinity has also been determined in vitro using conventional filtration techniques by measuring the compounds' ability to displace [³H]-substance P SP from recombinant human NK₁ receptors expressed in CHO cell membranes prepared as described above. Briefly, ligand binding was performed in 0.2 ml of 50 mM HEPES, pH 7.4, containing 3 mM MnCl₂, 0.02% BSA, 0.5 nM [³H]-Substance P (30-56 Ci/mmol Amersham), a final membrane protein concentration of 30-50 μg/ml, and the test compounds. The incubation proceeded at room temperature for 40 min and was stopped by filtration. Non-specific binding was determined using excess of substance P (1 μM) and represents about 6-10% of the total binding.

Preferred compounds of the invention were further characterised in a functional assay for the determination of their effect to inhibit the intracellular calcium increase induced by SP in Human-NK₁—CHO cells using FLIPR technology. Briefly, after 30 minutes incubation with the cytoplasmic calcium indicator Fluo-4 AM (2 μM), cells were washed and incubated in the absence or presence of three or more different concentrations of antagonist for 60 minutes, at 37° C. in Hank's balanced salts with 20 mM Hepes, and then non-cumulative concentration-response curves of SP (2 pM-300 nM) was performed. The potency of the antagonist (pKₐ value) was calculated from Schild's analysis.

The action of the compounds of the invention at the NK₁ receptor and/or serotonin transporter may be determined by using conventional animal models.

Thus, the ability to bind at the NK₁ receptor and/or serotonin transporter was determined using the guinea pig pup isolation calls model as described by Pettijohn, Psychol. Rep., 1979 and Rupniak et al., Neuropharmacology, 2000.

The anti-anxiety activity obtained by the administration of a compound according to the invention can be demonstrated in the gerbil social interaction model, according to the method described by Cheeta et al. (Cheeta S. et al., 2001. Brain Research 915: 170-175).

SERT binding affinity has been determined in vitro by the compounds' ability to displace [3H]-citalopram from hSERT-LLCPK cell membranes. For the binding reaction, a final concentration of 0.25 nM of [³H] citalopram (84 Ci/mmol, Amersham) were incubated with 3-5 μg/ml of cell membrane and the compound to be tested at different concentrations (7 concentration points in duplicate) in 50 mM Tris HCl, pH 7.7, containing 120 mM NaCl, 5 mM KCl, 10 μM pargyline and 0.1% ascorbic acid. The reaction was performed for 120 min at 22° C. and was terminated through GF/B Unifilter (pre-soaked in 0.5% PEI) using a Cell Harvester (Tomtec). Scintillation fluid was added to each filtered spot and radioactivity was determined using a scintillation counter (TopCount (Packard)). Non-specific binding was determined using paroxetine (10 μM) and represents about 2-5% of the total binding. Competition experiments were conducted with duplicate determination for each point. Msat601 software package was used to elaborate the competition binding data. IC₅₀ values were converted to Kᵢ values using the Cheng-Prusoff equation and by using the K_{D} of [³H]citalopram determined in separate experiments.

For preferred compounds of the invention, the inhibitory activity of the compounds at the human serotonin transporter (hSERT) has been determined in vitro using porcine LLCPK cells (ATCC.) stably transfected with the hSERT (hSERT-LLCPK). The cells have been plated onto 96-well plates (10000 cells/well). After 24 hr, cells have been washed in uptake buffer (Hank's balanced salt solution+20 mM Hepes) and pre-incubated for 10 minutes at 30° C. with 50 μl of buffer containing the test compounds. 50 μl of 50 nM [³H] Serotonin (5-HT) solution (final concentration: 25 nM [³H] 5-HT) have been added and plates have been incubated for 7 min at 30° C., during which cells take up radiolabelled 5-HT. Aspirating the solution and rapidly washing the cells with cold buffer has terminated the uptake. The amount of radioactive 5-HT incorporated in the cells has then been measured by adding the scintillation cocktail directly onto the cells and reading the plate in the Top Count. The data have been digitally processed to obtain the pIC₅₀ values of the uptake inhibitors.

Compounds of the invention are useful in the treatment of CNS disorders and psychotic disorders, in particular in the treatment or prevention of depressive states and/or in the treatment of anxiety as defined in, but not restricted to, Diagnostic Statistical of Mental Disorder (DSM) IV edition edit by American Psychiatric Association and International Classification Diseases 10th revision (ICD10).

Thus, for example, depressive states depression includes depressive mood episodes, depressive disorders, bipolar disorders, other mood, psychotic, adjustment disorders, premenstrual and dysphroic disorder (PMDD). Thus, for example, depressive mood episodes include major depressive episodes and mixed episodes. Depressive disorders include Major Depressive Disorder (MDD), single or recurrent episodes (with or without psychotic features, catatonic features, melancholic features, atypical features, anxious depression, or postpartum onset), dysthymic disorder (with early or late onset and with or without atypical features) and depressive disorder not otherwise specified. Bipolar disorders include bipolar I and II disorders, cyclothymic disorder and bipolar disorder not otherwise specified. Other mood, psychotic and adjustment disorders include neurotic depression; mood disorders due to general medical conditions including, but not limited to, myocardial infarction, diabetes, miscarriage, abortion, dementia of the Alzheimer's type (with early or late onset) with depressed mood, vascular dementia with depressed mood; substance-induced mood disorders including, but not limited to, depression induced by alcohol, amphetamines, cocaine, hallucinogens, inhalants, opioids, phencyclidines, sedatives, hypnotics, anxiolytics and other substances; schizoaffective disorder of the depressed type; adjustment disorder with depressed mood; adjustment disorder with mixed anxiety and depressed mood.

The term anxiety includes panic attacks, agoraphobia, anxiety disorders, adjustment disorders and separation anxiety disorder and premenstrual dysphroic disorder (PMDD). Thus, for example, anxiety disorders include panic disorder with or without agoraphobia, agoraphobia without a history of panic disorder, specific phobia, social phobia (social anxiety disorder), obsessive-compulsive disorder, Acute and posttraumatic stress disorders, generalised anxiety disorders, anxiety disorder due to a general medical condition, substance-induced anxiety disorder, anxiety disorder not otherwise specified and mixed anxiety-depression disorders. Adjustment disorders include adjustment disorder with anxiety and adjustment disorder with mixed anxiety and depressed mood. Compounds of the invention are useful as analgesics. In particular, they are useful in the treatment of traumatic pain such as postoperative pain; traumatic avulsion pain such as brachial plexus; chronic pain such as arthritic pain such as occurring in osteo-, rheumatoid or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, chemotherapy-induced neuropathy, AIDS related neuropathy, occipital neuralgia, geniculate neuralgia, glossopharyngeal neuralgia, reflex sympathetic dystrophy, phantom limb pain; various forms of headache such as migraine, acute or chronic tension headache, temporomandibular pain, maxillary sinus pain, cluster headache; odontalgia; cancer pain; pain of visceral origin; gastrointestinal pain; nerve entrapment pain; sport's injury pain; dysmennorrhoea; menstrual pain; meningitis; arachnoiditis; musculoskeletal pain; low back pain e.g. spinal stenosis; prolapsed disc; sciatica; angina; ankylosing spondyolitis; gout; burns; scar pain; itch and thalamic pain such as post stroke thalamic pain.

Compounds of the invention are also useful in the treatment of sleep disorders or sleep disturbances including dysomnia, insomnia, sleep apnea, narcolepsy, and circadian ritmic disorders or in the treatment of sleep disorders and/or sleep disturbances related or due to other disorders.

Compounds of the invention are also useful in the treatment or prevention of the cognitive disorders. Cognitive disorders include dementia, amnestic disorders and cognitive disorders not otherwise specified.

Furthermore, compounds of the invention are also useful as memory and/or cognition enhancers in healthy humans with no cognitive and/or memory deficit.

Compounds of the invention are also useful in the treatment of tolerance to and dependence on a number of substances. For example, they are useful in the treatment of dependence on nicotine, alcohol, caffeine, phencyclidine (phencyclidine like compounds) or in the treatment of tolerance to and dependence on opiates (e.g. cannabis, heroin, morphine) or benzodiazepines; in the treatment of addiction to cocaine, sedative ipnotic, amphetamine or amphetamine-related drugs (e.g. dextroamphetamine, methylamphetamine) or a combination thereof.

Compounds of the invention are also useful as anti-inflammatory agents. In particular, they are useful in the treatment of inflammation in asthma, influenza, chronic bronchitis and rheumatoid arthritis; in the treatment of inflammatory diseases of the gastrointestinal tract such as Crohn's disease, ulcerative colitis, inflammatory bowel disease and non-steroidal anti-inflammatory drug induced damage; inflammatory diseases of the skin such as herpes and eczema; inflammatory diseases of the bladder such as cystitis and urge incontinence; and eye and dental inflammation.

Compounds of the invention are also useful in the treatment of allergic disorders, in particular allergic disorders of the skin such as urticaria, and allergic disorders of the airways such as rhinitis.

Compounds of the invention are also useful in the treatment or prevention of schizophrenic disorders including paranoid schizophrenia, disorganised schizophrenia, catatonic schizophrenia, undifferentiated schizophrenia, residual schizophrenia.

Compounds of the invention are also useful in the treatment of emesis, i.e. nausea, retching and vomiting. Emesis includes acute emesis, delayed emesis and anticipatory emesis. The compounds of the invention are useful in the treatment of emesis however induced. For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiation of the thorax or abdomen, such as in the treatment of cancer; poisons; toxins such as toxins caused by metabolic disorders or by infection, e.g. gastritis, or released during bacterial or viral gastrointestinal infection; pregnancy; vestibular disorders, such as motion sickness, vertigo, dizziness and Meniere's disease; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); opioid analgesics, such as morphine; and gastro-oesophageal reflux disease (GERD) such as erosive GERD and symptomatic GERD or non erosive GERD, acid indigestion, over-indulgence of food or drink, acid stomach, sour stomach, waterbrash/regurgitation, heartburn, such as episodic heartburn, nocturnal heartburn, and meal-induced heartburn, dyspepsia and functional dyspepsia.

Compounds of the invention are also useful in the treatment of gastrointestinal disorders such as irritable bowel syndrome, gastro-oesophageal reflux disease (GERD) such as erosive GERD and symptomatic GERD or non erosive GERD, acid indigestion, over-indulgence of food or drink, acid stomach, sour stomach, waterbrash/regurgitation, heartburn, such as episodic heartburn, nocturnal heartburn, and meal-induced heartburn, dyspepsia and functional dyspepsia (such as ulcer-like dyspepsia, dysmotility-like dyspepsia and unspecified dyspepsia) chronic constipation; skin disorders such as psoriasis, pruritis and sunburn; vasospastic diseases such as angina, vascular headache and Reynaud's disease; cerebral ischeamia such as cerebral vasospasm following subarachnoid haemorrhage; fibrosing and collagen diseases such as scleroderma and eosinophilic fascioliasis; disorders related to immune enhancement or suppression such as systemic lupus erythematosus and rheumatic diseases such as fibrositis; and cough.

The compounds of the invention are also useful in premenstrual dysphoric disorder (PMDD), in chronic fatigue syndrome and Multiple sclerosis.

Compounds of the invention have been found to exhibit anxiolytic and antidepressant activity in conventional tests. For example, in Guinea pig pups separation-induced vocalisations (Molewijk et al., 1996) and in the gerbil social interaction model, according to the method described by Cheeta et al. (Cheeta S. et al., 2001. Brain Research 915: 170-175).

The invention therefore provides a compound of formula (I) or a pharmaceutically acceptable salt or solvate thereof for use in therapy, in particular in human medicine.

There is also provided as a further aspect of the invention the use of a compound of formula (I) or a pharmaceutically acceptable salt or solvate thereof in the preparation of a medicament for use in the treatment of conditions mediated by tachykinins (including substance P and other neurokinins) and/or by selective inhibition of serotonin reuptake.

There is also provided as a further aspect of the invention the use of a compound of formula (I) or a pharmaceutically acceptable salt or solvate thereof in the treatment of conditions mediated by tachykinins (including substance P and other neurokinins) and/or by selective inhibition of the serotonin reuptake transporter protein.

In a further aspect there is provided the use of a compound of formula (I) or a pharmaceutically acceptable salt or solvate thereof in the preparation of a medicament for use in the treatment of depression and/or anxiety.

In a further aspect there is provided the use of a compound of formula (I) or a pharmaceutically acceptable salt or solvate thereof in the treatment of depression and/or anxiety.

In an alternative or further aspect there is provided a method for the treatment of a mammal, including man, in particular in the treatment of conditions mediated by tachykinins, including substance P and other neurokinins and/or by selective inhibition of the serotonin reuptake transporter protein comprising administration of an effective amount of a compound of formula (I) or a pharmaceutically acceptable salt thereof.

In a further aspect of the present invention there is provided a method for the treatment of a mammal, including man, in particular for the treatment of depression and/or anxiety which method comprises administration of an effective amount of a compound of formula (I) or a pharmaceutically acceptable salt or solvate thereof.

It will be appreciated that reference to treatment is intended to include prophylaxis as well as the alleviation of established symptoms.

Compounds of formula (I) may be administered as the raw chemical but the active ingredient is preferably presented as a pharmaceutical formulation.

Accordingly, the invention also provides a pharmaceutical composition which comprises at least one compound of formula (I) or a pharmaceutically acceptable salt thereof and formulated for administration by any convenient route. Such compositions are preferably in a form adapted for use in medicine, in particular human medicine, and can conveniently be formulated in a conventional manner using one or more pharmaceutically acceptable carriers or excipients.

Thus, compounds of formula (I) may be formulated for oral, buccal, parenteral, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or formulated in conventional manner.

The compounds of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa buffer or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device.

A proposed dose of the compounds of the invention is 1 to about 1000 mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus, for parenteral administration a daily dose will typically be in the range of 1 to about 100 mg, preferably 1 to 80 mg per day. For oral administration a daily dose will typically be within the range 1 to 300 mg e.g. 1 to 100 mg.

Compounds of formula (I), and salts and solvates thereof, may be prepared by the general methods outlined hereinafter. In the following description, the groups R, R₁, R₂, R₃, R₄, R₅, R₆, R₇, R₈, R₉, R₁₀, m, n, p and q have the meaning as previously defined for compounds of formula (I) unless otherwise stated.

Compounds of formula (I) may be prepared by reaction of an activated derivative of the carboxylic acid (II), wherein R₆ is a nitrogen protecting group or (CH₂)qR₈, with amine (III)

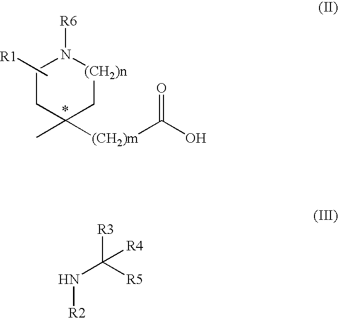

wherein R₂ is hydrogen, C₁₋₄ alkyl or nitrogen protecting group, followed where necessary by removal of any nitrogen protecting group.

Suitable activated derivatives of the carboxyl group include the acyl halide, mixed anhydride, activated ester such as thioester or the derivative formed between the carboxylic acid group and a coupling agent such as that used in peptide chemistry, for example carbonyl diimidazole or dicyclohexylcarbodiimide.

The reaction is preferably carried out in an aprotic solvent such as hydrocarbon, halohydrocarbon such as dichloromethane or an ether such as tetrahydrofuran.

The activated derivatives of the carboxylic acid (II) may be prepared by conventional means. A particular suitable activated derivative for use in this reaction is O-(Benzotriazol-1-yl)-N,N,N′,N′-tetramethyluronium tetrafluoroborate.

The reaction is suitably carried out in a solvent such as NN-dimethylformamide.

Compounds of formula (I), wherein R₂ is C₁₋₄ alkyl may be prepared by reaction of a compound of formula (I), in which R₂ is hydrogen, with (C₁₋₄ alkyl)L wherein L is a suitable leaving group selected from iodine, bromine in the presence of a base, conveniently in the presence of an inorganic base (e.g sodium hydride).

The reaction is conveniently carried out in a solvent such as N,N-dimethylformamide or tetrahydrofuran.

Compounds of formula (II), wherein m is 1 may be prepared by reaction of a derivative (IV), wherein R₁₁ is CH(CN)CO₂R₁₂ in which R₁₂ is a suitable carboxyl protecting group,

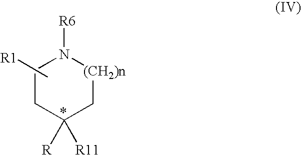

with an acid such as for example concentrated sulfuric acid, followed (if it is still necessary) by removal of the carboxyl protecting group R₁₂.

The reaction is conveniently carried out in a solvent such as acetic acid and heating the reaction mixture up to 150°.

Alternatively compounds of formula (II), wherein m is 1, may be prepared by reaction of a derivative (IV), wherein R₁₁, is the group (V),

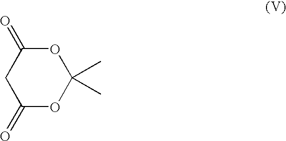

in 3-pentanone and water by heating the reaction mixture to reflux. Alternatively, the reaction can be carried out in the presence of an acid such as for example hydrochloric acid and a solvent such as tetrahydrofuran by heating the reaction mixture to reflux.

Compounds of formula (II), wherein m is zero, may be prepared by hydrolysis of a cyano derivative (VI) in the presence of a base such as alkaline base (i.e potassium hydroxide).

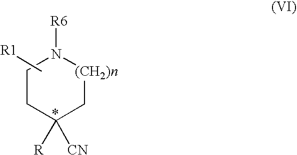

The reaction is suitably carried out in aqueous solvent and with heating.

Compounds of formula (IV), wherein m is 1, R₁₁ is CH(CN)CO₂R₁₂, in which R₁₂ is a suitable carboxyl protecting group, may be prepared by reaction of a compound of formula (VII) with a R-MgL (VIII), wherein L is a halogen group (i.e bromine)

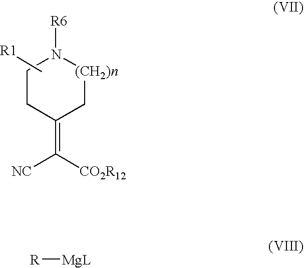

The reaction conveniently takes place in an aprotic solvent such as a hydrocarbon (e.g toluene), ethers (e.g tetrahydrofuran) and at a temperature within the range 0-25° C., optionally in the presence of Cupper(I) salts such as for example Cupper Iodide.

Suitable carboxyl protecting groups R₁₂ for use in the above reactions include alkyl, such as methyl or ethyl, trichloroalkyl, trialkylsilylalkyl, or arylmethyl groups such as benzyl, nitrobenzyl or trityl.

Compounds of formula (IV), wherein R₁₁ is the group (V) may be prepared by reaction of a compound of formula (IX)

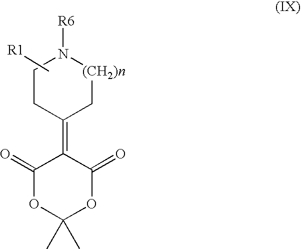

with a compound of formula (VIII), wherein L is a halogen group (e.g bromine) or a compound of RW(VIIIa) in which W is an alkali metal base such as for example lithium or magnesium.

The reaction conveniently takes place in an aprotic solvent such as a hydrocarbon (e.g toluene), ethers (e.g tetrahydrofuran) and at a temperature within the range −80-25° C., optionally in the presence of Cupper(I) salts such as for example Cupper Iodide.

Compounds of formula (VII) may be prepared by reaction of a compound of formula (X) with a cyano derivative (XI) wherein R₁₂ has the meaning defined above.

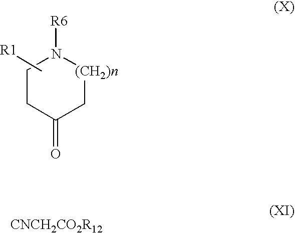

Compounds of formula (IX) may be prepared by reaction of a compound of formula (X) with the derivative (V).

Compounds of formulae (VI) and (X) may be prepared with analogous methods to those used for known compounds. Thus, compounds of formula (VI) may be prepared according to the procedure described in Cammack et al., Heterocyclic 23, 73 (1986).

Compounds of formula (X) may be prepared according to the procedure described in WO 2001/000206.

When R₆ and/or R₂ are a nitrogen protecting group, examples of suitable groups include alkoxycarbonyl e.g. t-butoxycarbonyl, benzyloxycarbonyl, arylsulphonyl e.g. phenysulphonyl or 2-trimethylsilylethoxymethyl.

Protection and deprotection may be effected using conventional techniques such as those described in “Protective Groups in Organic Synthesis 2^{nd} Ed.” by T. W. Greene and P. G. M. Wuts (John Wiley and Sons, 1991) and as described in the examples hereinafter.

When a specific enantiomer or diastereoisomer of a compound of general formula (I) is required, this may be obtained for example by resolution of a corresponding enantiomeric or diastereoisomeric mixture of a compound of formula (I) using conventional methods. Thus, for example, specific enantiomers or diastereoisomers of the compounds of formula (I) may be obtained from the corresponding enantiomeric or diastereoisomeric mixture of a compound of formula (I) using chiral chromatographic methods such as for example chiral HPLC or chiral SFC (Supercritical Fluid Chromatography).

Alternatively a specific enantiomer or diastereoisomer of a compound of general formula (I) may be synthesised from the appropriate optically active intermediates using any of the general processes described herein.

Thus, in one embodiment of the invention a specific enantiomer or diastereoisomer of compounds of formula (I) may be prepared by reaction of a chiral amine (III) using any of the processes described above for preparing compounds of formula (I) from amine (III). Thus, for example a specific diastereoisomer of compounds of formula (I), wherein R₂ is hydrogen and R₃ and R₄ are not the same group, may be obtained by reaction of a syn or anti isomer of a compound of formula (II) with a chiral amine of formula (III), wherein R₃ and R₄ are not the same group.

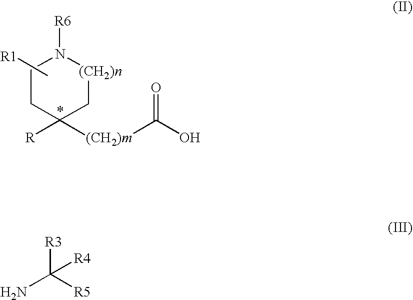

The chiral amine (III) may be prepared from the corresponding racemic amine (III) using any conventional procedures such as salt formation with a suitable optically active acid such as for example (S)-methoxyphenylacetic acid or (R)-methoxyphenylacetic acid, or using chiral HPLC procedure.

Where it is desired to isolate a compound of formula (I) as a salt, for example a pharmaceutically acceptable salt, this may be achieved by reacting a compound of formula (I) in the form of the free base with an appropriate amount of suitable acid and in a suitable solvent such as an alcohol (e.g. ethanol or methanol), an ester (e.g. ethyl acetate) or an ether (e.g. diethyl ether, tert-butylmethyl ether or tetrahydrofuran).

In the Intermediates and Examples unless otherwise stated:

Melting points (m.p.) were determined on a Buchi m.p. apparatus and are uncorrected. rt refers to room temperature. Infrared spectra (IR) were measured in chloroform or nujol solutions on a FT-IR instrument. Proton Magnetic Resonance (NMR) spectra were recorded on Varian instruments at 300, 400 or 500 MHz, on Bruker instrument at 300 MHz, chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at temperature ranging from 25 to 90° C.; when more than one conformer was detected the chemical shifts for the most abundant one is reported. Mass spectra (MS) were taken on a 4 II triple quadrupole Mass Spectrometer (Micromass UK) or on a Agilent MSD 1100 Mass Spectrometer, operating in ES (+) and ES (−) ionization mode or on a Agilent LC/MSD 1100 Mass Spectrometer, operating in ES (+) and ES (−) ionization mode coupled with HPLC instrument Agilent 1100 Series [LC/MS-ES (+): analysis performed on a Supelcosil ABZ+Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [CH₃CN] in 5 min, finally under these conditions for 2 min; T=40° C.; flux=1 mL/min; LC/MS-ES (−): analysis performed on a Supelcosil ABZ+Plus (33×4.6 mm, 3 μm) (mobile phase: 100% [water+0.05% NH₃] for 1 min, then from 100% [water+0.05% NH₃ to 5% [water+0.05% NH₃] and 95% [CH₃CN] in 5 min, finally under these conditions for 2 min; T=40° C.; flux=1 mL/min]. In the mass spectra only one peak in the molecular ion cluster is reported. Optical rotations were determined at 20° C. with a Jasco DIP360 instrument (I=10 cm, cell volume=1 mL, λ=589 nm). Flash silica gel chromatography was carried out over silica gel 230-400 mesh supplied by Merck AG Darmstadt, Germany or over Varian Mega Be—Si pre-packed cartridges or over pre-packed Biotage silica cartridges.

HPLC (walk-up) refers to HPLC analysis performed on a Luna C18 (mobile phase: from 100% [water+0.05% TFA] to 5% [water+0.05% TFA] and 95% [CH₃CN+TFA 0.05%] in 8 min; T=40° C.; flux=1 mL/min).

T.I.c. refers to thin layer chromatography on 0.25 mm silica gel plates (60F-254 Merck) and visualized with UV light. For phase separations performed by using microfiltration devices: phase separation cartridge with polypropylene frit by Whatman or Alltech. SCX means: SCX-cartridges (loading 0.75 mmol\g) by Varian.

Solutions were dried over anhydrous sodium sulphate.

Methylene chloride was redistilled over calcium hydride and tetrahydrofuran was redistilled over sodium.

The following abbreviations are used in the text: AcOEt=ethyl acetate, CH=cyclohexane, DCM=methylene chloride, DIPEA=N,N-diisopropylethylamine, DMF=N,N′-dimethylformamide, Et2O=diethyl ether, EtOH=ethanol, MeOH=methanol, TEA=triethylamine, THF=tetrahydrofuran, TFA=trifluoroacetic acid, CH3CN=acetonitrile, TBTU=O-(benzotriazol-1-yl)-N,N,N′N′-tetramethyluronium tetrafluoroborate, std=saturated.

In the text:

Enantiomer 1 or Enantiomer 2 refers to a single enantiomer whose absolute stereochemistry was not characterised.

Chain enantiomer I or chain enantiomer 2 refers to a compound of the invention or an intermediate thereof wherein R₃ and R₄ are not the same group, having a single but not determinated configuration at the carbon atom shown as ** in the formula (Ia)

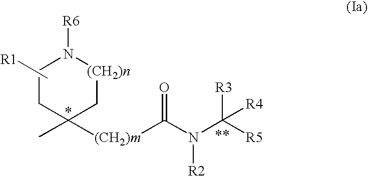

For compounds of the invention wherein m is 1, anti isomer refers to compounds of the invention or intermediate thereof in which the group R₁ is different from hydrogen and wherein the configuration of the carbon atom to which the group R₁ is attached and the configuration of the carbon atom shown as * are represented by formula 1(b) and 1(c).

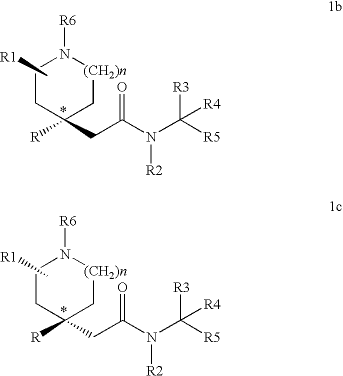

For compounds of the invention wherein m is 1, syn isomer refers to compounds of the invention or intermediate thereof in which the group R₁ is different from hydrogen and wherein the configuration of the carbon atom to which the group R₁ is attached and the configuration of the carbon atom shown as * are represented by formula 1(a) and 1(d).

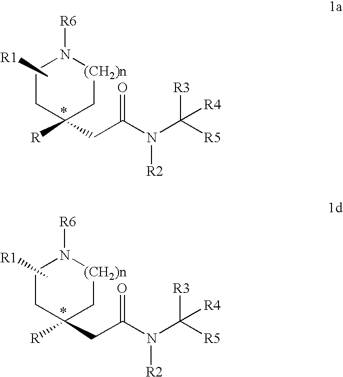

Syn isomer 1 or Syn isomer 2 refers to a single isomer having formula (1a) or (1d)

### Intermediate 1

### Methyl 4-bromo-7-fluoro-2-naphthalenecarboxylate

Isoamylnitrite (9.8 mL) dissolved in dimethoxyethane (59 mL) and a solution of 2-amino-4-fluorobenzoic acid (11.5 g) in dimethoxyethane (59 mL) were both added in separate streams at matching rate over 40 min to a refluxing solution of 3-bromo-coumalic acid methyl ester (3.3 g) in dimethoxyethane (55 mL) and catalytic amount of trichloroacetic acid (30 mg). The reaction mixture was heated under reflux for a further 1 h after the end of the additions in order to ensure complete reaction. Then the temperature was decreased to 50° C. and toluene (77 mL) was added. The mixture was then cooled to rt, the phases were separated and the organic one was extracted with aqueous 2M NaOH (110 mL), aqueous 5% sodium bisolfite (110 mL), water (110 mL), aqueous 2M HCl (110 mL) and finally water (110 mL).

Solvent was then removed by evaporation under reduced pressure to give a crude which was purified by Biotage Flash Chromatography eluting with CH:AcOEt=9:1 to give the title compound (650 mg) as a yellow oil.

NMR (d₆-DMSO): δ (ppm) 8.66 (s, 1H); 8.23 (dd, 1H); 8.18 (d, 1H); 8.09 (dd, 1H); 7.73 (dt, 1H); 3.89 (s, 3H).

### Intermediate 2

### 4-bromo-7-fluoro-2-naphthalenecarboxylic Acid

Intermediate 1 (970 mg) was dissolved in THF (20 mL) and water (10 mL) and then LiOH.H₂O (577 mg) was added. The mixture was heated at 80° C. for 2 h. Then it was cooled to rt and aqueous 2M HCl was added. The aqueous phase was extracted with AcOEt and the organic extracts were dried and evaporated under vacuum to give the title compound (850 mg) as a yellow solid.

NMR (d₆-DMSO): δ (ppm) 13.4 (bs, 1H); 8.63 (s, 1H); 8.23 (dd, 1H); 8.18 (s, 1H); 8.07 (dd, 1H); 7.71 (td, 1H).

### Intermediate 3

### 4-bromo-7-fluoro-N-hydroxy-2-naphthalenecarboxamide

Intermediate 2 (850 mg) was dissolved in DMF (3 mL) and then TBTU (1.32 g) and DIPEA (1.9 mL) were added. The mixture was stirred for 30 min under a nitrogen atmosphere and then hydroxylamine hydrochloride (286 mg) was added; after stirring for 2 h aqueous std NH₄Cl was added and the aqueous phase was extracted with AcOEt. The organic phase was then washed with aqueous std NaHCO₃, dried and evaporated under vacuum to give a crude which was triturated with pentane to afford the title compound (360 mg) as a withish solid.

MS (ES/+): m/z=284 [M+H]⁺.

### Intermediate 4

### 4-bromo-7-fluoro-2-naphthalenecarbonitrile

Intermediate 3 (360 mg) was suspended in fluoro benzene (11 mL) under nitrogen atmosphere at rt and phosphorous tribromide (358 μL) was dropped on the mixture over 5 min. The suspension was refluxed at 80° C. for 18 h; then it was cooled to rt and aqueous std NaHCO₃ was added and the aqueous phase extracted with AcOEt. The organic extracts were collected, dried and evaporated under vacuum to give a crude which was purified by biotage flash cromathography eluting with CH:AcOEt=98:2 to afford the title compound (200 mg) as a pale brown solid.

NMR (d₆-DMSO): δ (ppm) 8.66 (s, 1H); 8.32 (dd, 1H); 8.28 (d, 1H); 8.01 (dd, 1H); 7.84 (dt, 1H).

### Intermediate 5

### 4-ethenyl-7-fluoro-2-naphthalenecarbonitrile

A solution of intermediate 4 (25 mg), TETRAKIS (triphenylphosphine) Palladium (0) (5 mg), tributyl(ethenyl)stannane (32 μL) and one crystal of hydroquinone in dry toluene (1 mL) was heated at 110° C. for 4 h. The mixture was then cooled to rt and aqueous std NaHCO₃ and AcOEt were added; the organic phase was separated, washed with aqueous 10% KF, dried and evaporated under vacuum to give the crude. It was then purified by flash chromatography eluting by CH:AcOEt=9:1, to give the title compound (14 mg) as a yellow solid.

NMR (d₆-DMSO): δ (ppm) 8.51 (s, 1H); 8.40 (dd, 1H); 7.98 (d, 1H); 7.92 (dd, 1H); 7.70 (td, 1H); 7.57 (dd, 3H); 6.07 (d, 1H); 5.65 (d, 1H).

### Intermediate 6

### 7-fluoro-4-formyl-2-naphthalenecarbonitrile

Intermediate 5 (14 mg) was dissolved in THF (1.5 mL) and water (0.3 mL); aqueous 4% osmium tetroxide solution (22 μL) and sodium periodate (30 mg) were added and the solution was vigorously stirred at rt and under nitrogen atmosphere for 4 h. Then a 5% solution of sodium methabisolfite in aqueous std NaHCO₃ was added; the organic phase was extracted with AcOEt, dried and evaporated under vacuum to give the title compound (14 mg) as a pale yellow solid.

NMR (d₆-DMSO): δ (ppm) 10.38 (s, 1H); 9.23 (dd, 1H); 8.90 (s, 1H); 8.50 (s, 1H); 8.03 (dd, 1H); 7.87 (td, 1H).

### Intermediate 7

### 7-fluoro-4-[(methylamino)methyl]-2-naphthalenecarbonitrile

Intermediate 6 (124 mg) was suspended in dry MeOH (6 mL) under a nitrogen atmosphere and then methylamine 2.0M solution in MeOH (1.6 mL) was added. The mixture was stirred at rt for 2 h; then potassium boron hydride (66 mg) was added in three portions and the solution was stirred for further 2 h. Aqueous std NH4Cl and AcOEt were added, the organic phase separated, dried and evaporated under vacuum to give a crude which was purified by SCX cartridge to afford the title compound (100 mg) as a yellow solid.

MS (ES/+): m/z=215 [M+H]⁺.

### Intermediate 8

### 3-bromo-N-methyl-N-(methyloxy)-1-naphthalenecarboxamide

A solution of 3-bromo-1-naphthalenecarboxylic acid (1 g), O-(7-Azabenzotriazol-1-yl)-N,N,N′,N′-tetramethyluronium hexafluorophosphate (1.97 g) and DIPEA (2.35 mL) in anhydrous DMF (5 ml) was stirred at rt for 30 min under a Nitrogen atmosphere. N,O-dimethylhydroxylamine hydrochloride (465 mg) was added and the mixture stirred at rt for 2 h. The mixture was washed with aqueous 5% NaHCO₃, the organic layer was dried, concentrated in vacuo and the residue purified by flash chromatography (CH/AcOEt 2:8) to give the title compound (986 mg) as a white foam.

NMR (acetone-d₆): δ (ppm) 8.22 (s, 1H); 7.97-7.60 (m, 4H); 7.65 (d, 1H); 3.50 (bs, 3H); 3.38 (bs, 3H).

### Intermediate 9

### 1-(3-bromo-1-naphthalenyl)ethanone

Intermediate 8 (986 mg) was dissolved in dry THF (3 mL) at 0° C. under nitrogen atmosphere and then methyl magnesium bromide 3.0M solution in Et2O (2.8 mL) was added; the solution was stirred under these conditions for 2 h. Aqueous std NH4Cl and AcOEt were added, the organic phase separated, dried and evaporated under vacuum to give a crude which was purified by flash chromatography eluting with CH:AcOEt=9:1 to afford the title compound (753 mg) as a solid.

NMR (CDCl₃): δ (ppm) 8.6 (d, 1H); 8.2 (s, 1H); 8.0 (s, 1H); 7.8 (d, 1H); 7.6 (m, 2H); 2.8 (s, 3H).

### Intermediate 10

### 4-acetyl-2-naphthalenecarbonitrile

Intermediate 9 (367 mg) was dissolved in dry DMF (2.5 mL) and then pyridine (360 μl) and copper cyanide (396 mg) were added. The mixture was heated at 150° C. for 48 h.

Aqueous std NH4Cl, aqueous NH₄OH (1 mL), and AcOEt were added, the organic phase separated, dried and evaporated under vacuum to give a crude which was purified by flash cromathography eluting with CH:AcOEt=9:1 to afford the title compound (122 mg) as a yellow solid.

NMR (CDCl₃): δ (ppm) 8.7 (d, 1H); 8.4 (s, 1H); 8.0 (s, 1H); 7.9 (d, 1H); 7.8 (t, 1H); 7.6 (t, 1H); 2.8 (s, 3H).

### Intermediate 11

### 4-[1-(methylamino)ethyl]-2-naphthalenecarbonitrile

Intermediate 10 (210 mg) was suspended in dry MeOH (3 mL) under a nitrogen atmosphere and then methylamine 2.0M solution in MeOH (2.7 mL) was added. The mixture was stirred at rt overnight; then potassium boron hydride (59 mg) was added in three portions and the solution was stirred for further 1.5 h. Aqueous std NH4Cl and AcOEt were added, the organic phase separated, dried and evaporated under vacuum to give a crude which was purified by SCX cartridge to afford the title compound (128 mg) as a yellow oil.

MS (ES/+): m/z=211 [M+H]⁺.

### Intermediate 12 and Intermediate 13

### 4-[1-(methylamino)ethyl]-2-naphthalenecarbonitrile (Enantiomer 1)

### 4-[1-(methylamino)ethyl]-2-naphthalenecarbonitrile Enantiomer 2)

To a solution of intermediate 11 (2.6 g) in acetone (18 mL), a solution of (S)-methoxyphenylacetic acid (2.0 g) in acetone (18 mL) was added. The thick suspension was heated at 56° C. for 40 minutes then it was stirred at rt overnight. The slurry was filtered and the solid residue (2.15 g) was triturated in acetone (12 mL) by heating to reflux 1 h and cooling to rt. The suspension was filtered and the solid residue (1.8 g) was triturated again twice as described above with acetone to give (S)-methoxyphenylacetic acid salt of 4-[1-(methylamino)ethyl]-2-naphthalenecarbonitrile (1.3 g). The solid was stirred in a mixture of aqueous 1 μM NaOH (20 mL) and DCM (20 mL). The organic phase was washed with brine (20 mL), dried and concentrated in vacuo to give the title compound intermediate 12 (0.760 g) as a colourless oil.

The mother liquors from the precipitation and first trituration were collected, concentrated in vacuo, treated with aqueous 1 M NaOH (20 mL) and extracted with DCM (20 mL). The organic phase was dried and concentrated in vacuo to give a colourless oil (1.49 g); it was then treated with (R)-methoxyphenylacetic acid (1.18 g) in acetone (2×5 mL) as described above (one precipitation and two triturations) to give (R)-methoxyphenylacetic acid salt of 4-[1-(methylamino)ethyl]-2-naphthalenecarbonitrile (1.2 g). This solid was stirred in a mixture of aqueous 1 M NaOH (10 mL) and DCM (10 mL). The organic phase was washed with brine (20 mL), dried and concentrated in vacuo to give the title compound intermediate 13 (0.720 g) as colourless oil.

### Intermediate 12 (Enantiomer 1):

NMR (CDCl₃): δ (ppm) 8.22 (d, 1H); 8.13 (s, 1H); 7.93 (dd, 1H); 7.84 (d, 1H); 7.66 (td, 1H); 7.59 (td, 1H); 4.50 (q, 1H); 2.4 (s, 3H); 1.48 (d, 3H).

MS (ES/+): m/z=211 [M+H]⁺.

[α]_{D}=+119.6 (c=0.98, CH₃CN)

HPLC analytical conditions: column: Chiralcel OD 5 μM, 25×4.6 mm; mobile phase: A: n-hexane; B: Isopropanol+0.1% Isopropylamine; gradient isocratic 3% B; flow rate=1 mL/min; UV wavelength range: 200400 nm; analysis time: 30 min; retention time=14.6 minutes; purity (a/a %)=98.6%.

### Intermediate 13 (Enantiomer 2):

NMR (CDCl₃): δ (ppm) 8.22 (d, 1H); 8.13 (s, 1H); 7.93 (dd, 1H); 7.84 (d, 1H); 7.66 (td, 1H); 7.59 (td, 1H); 4.50 (q, 1H); 2.4 (s, 3H); 1.48 (d, 3H).

MS (ES/+): m/z=211 [M+H]⁺.

[α]_{D}=−118.6 (c=1.095, CH₃CN)

HPLC analytical conditions: column: Chiralcel OD 5 μM, 25×4.6 mm; mobile phase: A: n-hexane; B: Isopropanol+0.1% Isopropylamine; gradient isocratic 3% B; flow rate=1 mL/min; UV wavelength range: 200-400 nm; analysis time: 30 min; retention time=17.6 minutes; purity (a/a %)=98.4%.

### Intermediate 14

### [1-(3-Chloro-1-naphthalenyl)ethyl]amine

A solution of 3-chloro-naphthalenecarbaldehyde (1.93 g) in dry THF (12 mL) was added dropwise to lithium bis(trimethylsilyl)-amide 1M solution in THF (10.1 mL) at −30° C. under a Nitrogen atmosphere. The resulting yellow mixture was stirred under a Nitrogen atmosphere from −30° C. to −5° C. for 1 h, then it was cooled down to −60° C. and methyllithium 1.6M solution in Et2O (11 mL) was added keeping the internal temperature of the reaction mixture <−55° C.

The resulting dark violet reaction mixture was stirred for 40 minutes at −50° C. under a Nitrogen atmosphere, then it was carefully quenched at −50° C. with aqueous 2M HCl (30 mL) until pH=2. The reaction was concentrated in vacuo and the aqueous residue was washed with 1:1 CH/Et2O (50 mL). The separated aqueous phase was then made basic (pH=14) at 0° C. with NaOH pellets. This basic aqueous phase was extracted with Et2O (3×60 mL), the collected organic layers were dried and concentrated in vacuo to give the title compound (1.12 g) as a yellow oil.

T.I.c.: AcOEt/MeOH 8:2, Rf=0.25 (detection with ninhydrine).

NMR (d₆-DMSO): δ (ppm) 8.14 (dd, 1H); 7.94-7.85 (m, 2H); 7.73 (d, 1H); 7.58-7.50 (m, 2H); 4.80 (q, 1H); 1.35 (d, 3H).

MS (ES/+): m/z=189 [M-NH₂]⁺.

### Intermediate 15 and Intermediate 16

### [1-(3-Chloro-1-naphthalenyl)ethyl]amine (Enantiomer 2) and [1-(3-chloro-1-naphthalenyl)ethyl]amine Enantiomer 1)

To a solution of intermediate 14 (1.12 g) in acetone (10 mL), a solution of (S)-methoxyphenylacetic acid (0.9 g) in acetone (10 mL) was added. The thick suspension was heated at 56° C. for 40 minutes then it was stirred at rt overnight. The slurry was filtered and the solid residue washed with acetone (10 mL). The solid (0.87 g) was triturated in acetone (10 mL) by heating to reflux for 1 h, cooling to rt and stirring overnight. The suspension was filtered and the solid residue (0.6 g) washed with acetone (10 mL) and triturated once again as described above to give (S)-methoxyphenylacetic acid salt of [1-(3-chloro-naphthalen-1-yl)-ethyl]amine (0.45 g). The solid was stirred in a mixture of aqueous std NaHCO₃ (20 mL) and DCM (20 mL). The organic phase was washed with brine (20 mL), dried and concentrated in vacuo to give the title compound intermediate 15 (0.25 g) as a colourless oil.

The mother liquors from the precipitation and first trituration were collected, concentrated in vacuo, treated with aqueous std NaHCO₃ (20 mL) and extracted with DCM (20 mL). The colourless oil thus obtained (1 g) was treated with (R)-methoxyphenylacetic acid (0.8 g) in acetone (8 mL) as described above (one precipitation and two triturations) to give (R)-methoxyphenylacetic acid salt of 1-(3-chloro-naphthalen-1-yl)-ethylamine (0.43 g). A portion of this solid (200 mg) was stirred in a mixture of aqueous std NaHCO₃ (10 mL) and DCM (10 mL). The organic phase was washed with brine (20 mL), dried and concentrated in vacuo to give the title compound intermediate 16 (0.100 g) as colourless oil.

### Intermediate 15 (Enantiomer 2):

NMR (d₆-DMSO): δ (ppm) 8.14 (dd, 1H); 7.94-7.85 (m, 2H); 7.73 (d, 1H); 7.58-7.50 (m, 2H); 4.80 (q, 1H); 1.35 (d, 3H).

MS (ES/+): m/z=189 [M-NH₂]⁺.

[α]_{D}=+69.7 (c=0.96, CH₃CN)

SFC (Gilson) analytical conditions: column: Chiralcel OD 25×4.6 mm; mobile phase: CO₂/Ethanol+0.1% Isopropanol 92/8 v/v; flow rate=2.5 mL/min; P=180 bar; T=35° C.; detection: λ=225 nm): retention time=13.8 minutes; purity (a/a %)>99%.

### Intermediate 16 (Enantiomer 1):

NMR (d₆-DMSO): δ (ppm) 8.14 (dd, 1H); 7.94-7.85 (m, 2H); 7.73 (d, 1H); 7.58-7.50 (m, 2H); 4.80 (q, 1H); 1.35 (d, 3H).

MS (ES/+): m/z=189 [M-NH₂]⁺.

[α]_{D}=−66.9 (c=1.065, CH₃CN)

SFC (Gilson) analytical conditions: column: Chiralcel OD 25×4.6 mm; mobile phase: CO₂/Ethanol+0.1% Isopropanol 92/8 v/v; flow rate=2.5 mL/min; P=180 bar; T=35° C.; detection: λ=225 nm): retention time=12.4 minutes; purity (a/a %)>99%.

### Intermediate 17

### 1,1-dimethylethyl [1-(3-chloro-1-naphthalenyl)ethyl]carbamate

### (Enantiomer 2)

Intermediate 15 (0.6 g) was dissolved in dry DCM (20 mL), then TEA (1.094 mL) and di-t-butyl-dicarbonate (820 mg) were added. The mixture was stirred overnight and then the solvent was removed under vacuum to give a crude which was purified by flash cromathography (eluting with CH:AcOEt=9:1) to afford the title compound (1.17 g) as a yellow oil.

T.I.c.: CH:AcOEt 9:1, Rf=0.32.

MS (ES/+): m/z=328 [M+Na]⁺.

### Intermediate 18

### 1,1-dimethylethyl [1-(3-chloro-1-naphthalenyl)ethyl]methylcarbamate

### (Enantiomer 2)

Intermediate 17 (1.16 g) was dissolved in dry DMF (7 mL), then NaH 60% dispersion in mineral oil (200 mg) was added under a nitrogen atmosphere and the mixture was stirred at rt for 15 min. Then methyl iodide (2.3 mL) was added and the solution was heated at 50° C. for 2 h. Water and AcOEt were added, the organic phase was separated, washed with brine, dried and evaporated under vacuum to give a crude which was purified by flash chromatography (eluting with CH:AcOEt=99:1 to 95:5) to afford the title compound (614 mg) as a yellow oil.

T.I.c.: CH/AcOEt 9:1, Rf=0.48.

MS (ES/+): m/z=342 [M+Na]⁺.

### Intermediate 19

### 1-(3-chloro-1-naphthalenyl)-N-methylmethanamine (Enantiomer 2)

To a solution of intermediate 18 (614 mg) in dry DCM (30 mL) at 0° C. and under nitrogen atmosphere, TFA (7.5 mL) was added and the solution was stirred under these conditions for 2 h. Then aqueous std NaHCO₃ solution was added, the organic phase separated, dried and evaporated under vacuum to give the title compound (446 mg) as a colourless oil

T.I.c.: DCM/MeOH 9:1, Rf=0.40.

MS (ES/+): m/z=189 [M-NHMe+H]⁺.

### Intermediate 20

### 7-formyl-1-benzofuran-5-carbonitrile

To a solution of 5-bromo-1-benzofuran-7-carbaldehyde (2.0 g) in DMF (15 mL) under Nitrogen atmosphere, pyridine (1.08 mL) and CuCN (1.2 g) were added. The mixture was heated and stirred at 140° C. for two days. An additional amount of CuCN (800 mg) was added and the mixture was stirred for further 4 h under these conditions. AcOEt was added, the solution was filtered on a gooch and washed three times with aqueous std NaHCO₃; the organic extracts were dried and evaporated under vacuum to give a crude which was purified by Biotage flash cromathography eluting with CH:AcOEt=4:1 to afford the title compound (400 mg) as a yellow solid.

T.I.c.: CH:AcOEt 7:3, Rf=0.26.

NMR (CDCl₃): δ (ppm) 10.45 (s, 1H); 8.15 (d, 1H); 8.07 (d, 1H); 7.89 (d, 1H); 6.95 (d, 1H).

### Intermediate 21

### 7-[(methylamino)methyl]-1-benzofuran-5-carbonitrile

Intermediate 20 (180 mg) was suspended in dry MeOH (2 mL) under a nitrogen atmosphere and then methylamine 2.0M solution in MeOH (2.1 mL) was added. The mixture was stirred at rt for 2 h; then potassium boron hydride (84 mg) was added and the solution was stirred overnight. AcOEt was added and the solution was washed with aqueous std NaHCO₃; the organic extracts were dried over and evaporated under vacuum to give a crude which was purified by SCX cartridge to afford the title compound (176 mg) as a white solid.

NMR (CDCl₃): δ (ppm) 7.78 (s, 1H); 7.65 (s, 1H); 7.45 (s, 1H); 6.79 (s, 1H); 3.99 (s, 2H); 2.44 (s, 3H).

MS (ES/+): m/z=187 [M+H]⁺.

### Intermediate 22

### 7-(1-hydroxyethyl)-1-benzofuran-5-carbonitrile

To a stirred solution of 5-bromo-1-benzofuran-7-carbaldehyde in dry THF (10 mL), cooled at −65° C. and under a nitrogen atmosphere, methyl magnesium bromide 3.0M solution in Et2O (1.52 mL) was added dropwise and the solution was stirred under these conditions for 2 h. Then aqueous std NH₄Cl was added and the mixture was extracted with AcOEt (3×20 mL). The organic extracts were collected, dried and evaporated under vacuum to give a crude which was purified by flash cromathography (eluting with CH:AcOEt=8:2) to afford the title compound (225 mg) as a yellow oil.

T.I.c.: CH:AcOEt 8:2, Rf=0.2.

NMR (CDCl₃): δ (ppm) 7.8 (s, 1H); 7.7 (s, 1H); 7.6 (d, 1H); 6.8 (d, 1H); 5.4 (d, 1H); 2.2 (m, 1H); 1.6 (m, 3H).

### Intermediate 23

### 7-acetyl-1-benzofuran-5-carbonitrile

To a solution of intermediate 22 (225 mg) in DCM (3 mL), Dess-Martin periodinane Reagent (561 mg) was added and the mixture was stirred for 2 h at rt under nitrogen atmosphere Aqueous std NaHCO₃ was added, together with aqueous 5% sodium thiosulfate solution, and the resulting mixture was stirred for 20 min; then it was extracted with DCM, dried and evaporated under vacuum to give a crude which was purified by Biotage flash chromatography eluting with CH:AcOEt=9:1 to afford the title compound (200 mg) as pale yellow oil.

NMR (CDCl₃): δ (ppm) 8.2 (s, 1H); 8.1 (s, 1H); 7.8 (s, 1H); 6.9 (s, 1H); 2.8 (s, 3H).

### Intermediate 24

### 7-[1-(methylamino)ethyl]-1-benzofuran-5-carbonitrile

Intermediate 23 (197 mg) was suspended in dry MeOH (8 mL) under a nitrogen atmosphere and then methylamine 2.0M solution in MeOH (2.7 mL) was added. The mixture was stirred at rt overnight; then potassium boron hydride (84 mg) was added and the solution was stirred at rt for 2 h. Water was added at 0° C., then MeOH was removed by evaporation under vacuum and the resulting aqueous phase was extracted with DCM; the organic extracts were collected, dried and evaporated under vacuum to give a crude which was purified by SCX cartridge to afford the title compound (183 mg) as a colourless oil.

NMR (CDCl₃): δ (ppm) 7.80 (s, 1H); 7.70 (s, 1H); 7.55 (s, 1H); 6.80 (s, 1H); 4.15 (q, 1H); 2.30 (s, 3H); 1.45 (d, 3H).

### Intermediate 25

### 1-(5-bromo-1-benzofuran-7-yl)ethanol

5-Bromo-1-benzofuran-7-carbaldehyde (800 mg) was dissolved in dry THF (50 mL) and to this solution, previously cooled at −78° C. and under nitrogen atmosphere, methyl magnesium bromide 3.0M solution in diethyl ether (2.4 mL) was slowly added. The solution was allowed to warm up to −50° C. and then aqueous NH4Cl std and AcOEt were added, the organic phase separated, washed with water and brine, and evaporated under vacuum to give a crude which was purified by Biotage flash cromathography eluting with CH:AcOEt=9:1 to afford the title compound (450 mg) as a yellow solid.

T.I.c.: CH:AcOEt 1:1, Rf=0.70.

### Intermediate 26

### [1-(5-bromo-1-benzofuran-7-yl)ethyl]methylamine

To a solution of intermediate 25 (450 mg) in CH₂Cl₂ (8 mL), Dess-Martin periodinane Reagent (800 mg) was added and the mixture was stirred for 1 h at rt under nitrogen atmosphere. Aqueous std NaHCO₃ was added, together with aqueous 5% sodium thiosulfate solution, and the resulting mixture was stirred for 20 min; then it was extracted with DCM dried and evaporated under vacuum to give the crude compound intermediate [T.I.c.: CH:AcOEt=7:3, Rf=0.5 (detection with 2,4-dinitrophenylhydrazine)]. This compound intermediate (250 mg) was suspended in dry methanol (5 mL) under a nitrogen atmosphere and then methylamine 2.0M solution in MeOH (2.6 mL) was added. The mixture was stirred at rt for 1 h; then potassium boron hydride (84 mg) was added and the solution was stirred at rt for 0.5 h. MeOH was removed by evaporation under vacuum and the crude was purified by SCX cartridge to afford the title compound as a pale yellow oil (130 mg).

MS (ES/+): m/z=254-256 [M+H]⁺.

### Intermediate 27

### [(5-bromo-1-benzofuran-7-VI)methyl]methylamine Hydrochloride

5-bromo-1-benzofuran-7-carbaldehyde (5 g) was suspended in dry MeOH (20 mL) under a nitrogen atmosphere and then methylamine 2.0M solution in MeOH (16.7 mL) was added. The mixture was stirred at rt for 1 h; then potassium boron hydride (1.79 g) was added and the solution was stirred for 30 min. MeOH was removed by evaporation under vacuum and DCM (300 mL) was added to dilute the crude; brine was used to wash the organic phase and then HCl 1.0M solution in diethyl ether was added (25 mL) to afford the title compound as a white solid (5 g).

MS (ES/+): m/z=240, 242 [M+H]⁺.

### Intermediate 28

### [(3-Chloro-1-naphthalenyl)methyl]methylamine

Methylamine (2M solution in MeOH—7 mL) was added to a solution of 3-chloro-naphthalene-1-carbaldeyde (750 mg) in MeOH (20 mL) under a Nitrogen atmosphere. The mixture was stirred at rt. for 2 hours, then it was cooled to 0° C. and potassium borohydride (290 mg) was added. The mixture was stirred at 0° C. for 2 hours, then it was quenched with water and extracted with DCM. The organic layer was dried, concentrated in vacuo and the residue purified on SCX-cartridge (loaded with DCM, washed with MeOH, eluted with NH3 0.25M solution in MeOH, followed by MeOH). Solvent evaporation gave the title compound (650 mg) as a yellow oil.

T.I.c.: AcOEt/MeOH 9:1, Rf=0.2 (detection with ninhydrine).

MS (ES/+): m/z=206 [M+H]⁺.

### Intermediate 29

### [(3-Bromo-1-naphthalenyl)methyl]methylamine

Methylamine 2M solution in MeOH (2.46 mL) was added to a solution of 3-bromo-naphthalene-1-carbaldehyde (290 mg) in anhydrous MeOH (12 mL) under a Nitrogen atmosphere and the solution was stirred at rt for 2 h. Potassium borohydride (100 mg) was added at 0° C. and the resulting mixture was stirred at rt overnight, then it was cooled to 0° C. and quenched by adding water (15 mL) and extracted with DCM (3×15 mL). The combined organic extracts were washed with brine, dried and concentrated in vacuo to give the title compound (288 mg) as a yellow oil.

T.I.c.: CH/AcOEt 3:7, Rf=0.1 (detection with ninhydrine).

MS (ES/+): m/z=250, 252 [M+H]⁺.

### Intermediate 30

### 4-Formyl-2-naphthalenecarbonitrile

To a solution of 4-(hydroxymethyl)-2-naphthalenecarbonitrile (87 mg) in anhydrous DCM (4 mL) Dess Martin periodinane (222 mg) was slowly added under a Nitrogen atmosphere. The reaction mixture was stirred at rt for 2 hours then it was diluted with Et2O (5 mL) and quenched by adding sodium thiosulphate (375 mg) dissolved in aqueous std NaHCO₃ (5 mL). The resulting mixture was stirred for additional 15 min then it was extracted with Et2O (3×5 mL). The combined organic extracts were dried and concentrated in vacuo to give the title compound (96.4 mg) as a white solid.

T.I.c: CH/AcOEt 6:4, Rf=0.7.

NMR (CDCl₃): δ (ppm) 10.40 (s, 1H); 9.25 (d, 1H); 8.45 (s, 1H); 8.10 (s, 1H); 8.00 (d, 1H); 7.85 (t, 1H); 7.75 (m, 1H).

MS (ES/+): m/z=182 [M+H]⁺.

### Intermediate 31

### 4-[(Methylamino)methyl]-2-naphthalenecarbonitrile

Methylamine 2.0M solution in MeOH (1.06 mL) was added to a solution of intermediate 6 (96 mg) in anhydrous MeOH (10 mL) under a Nitrogen atmosphere and the solution was stirred at rt for 2 h. Potassium borohydride (43.0 mg) was added at 0° C. and the resulting mixture was stirred at rt overnight, then it was cooled to 0° C. and quenched by adding water (5 mL) and extracted with DCM (3×5 mL). The combined organic extracts were washed with brine, dried and concentrated in vacuo. The residue was purified by SCX-cartridge (loaded with MeOH, washed with MeOH, eluted with NH3 0.25 M in MeOH) to give the title compound (79.5 mg) as a yellow oil.

T.I.c.: DCM/MeOH 8:2, Rf=0.61 (detection with ninhydrine).

MS (ES/+): m/z=197 [M+H]⁺.

### Intermediate 32

### 1,1-dimethylethyl 4-(1,3-benzodioxol-5-yl)-4-(2,2-dimethyl-4,6-dioxo-1,3-dioxan-5-yl)-1-piperidinecarboxylate

A solution of 5-bromo-1,3-benzodioxole (1.11 mL) in anhydrous THF (6 mL) was dropped into a suspension of magnesium turnings (250 mg) and few crystals of iodine in anhydrous THF (2.5 mL) under a Nitrogen atmosphere. The mixture refluxed for 30 minutes, then it was allowed to cool to rt and added drop-wise to a mixture of 1,1-dimethylethyl 4-(2,2-dimethyl-4,6-dioxo-1,3-dioxan-5-ylidene)-1-piperidinecarboxylate (1 g) and copper iodide (351 mg) in anhydrous THF (15 mL) previously cooled to 0° C. under a Nitrogen atmosphere. The mixture was allowed to warm to rt and stirred at 23° C. for 2 h. The mixture was treated with aqueous std NH4Cl solution and aqueous NH4OH (1 mL) and extracted with AcOEt. The combined organic extracts were collected, dried and concentrated under vacuum. The residue was purified by Biotage flash chromatography (CH/AcOEt 8:2) to give the title compound (720 mg) as a white foam.

T.I.c.: CH:AcOEt 1:1, Rf=0.52 (detection with ninhydrine).

MS (ES/+): m/z=470 [M+Na]⁺.

Following the same procedure described for intermediate 32, intermediate 33, 34, 35, 36, 37 were obtained.

### Intermediate 33

### 1,1-dimethylethyl 4-(2,2-dimethyl-4,6-dioxo-1,3-dioxan-5-yl)-4-[3-fluoro-4-(methyloxy)phenyl]-1-piperidinecarboxylate

Staring from 4-bromo-2-fluoro-1-(methyloxy)benzene (6 g) and using 1,1-dimethylethyl 4-(2,2-dimethyl-4,6-dioxo-1,3-dioxan-5-ylidene)-1-piperidinecarboxylate (4 g), 700 mg of the title compound were obtained.

T.I.c.: CH:AcOEt 1:1, Rf=0.41 (detection with ninhydrine).

MS (ES/−): m/z=450 [M−H]⁻.

### Intermediate 34

### 1,1-dimethylethyl 4-(2,2-dimethyl-4,6-dioxo-1,3-dioxan-5-yl)-4-(3-fluoro-4-methylphenyl)-1-piperidinecarboxylate

By adding (3-fluoro-4-methylphenyl)magnesiumbromide 0.5M solution in THF (24.6 mL) and starting from 1,1-dimethylethyl 4-(2,2-dimethyl-4,6-dioxo-1,3-dioxan-5-ylidene)-1-piperidinecarboxylate (2 g), 3.12 g of the title compound as a yellow foam were obtained without any chromatographic purification.

MS (ES/−): m/z=434 [M−H]⁻.

### Intermediate 35

### 1,1-dimethylethyl 4-(2,2-dimethyl-4,6-dioxo-1,3-dioxan-5-yl)-4-[4-(methyloxy)phenyl]-1-piperidinecarboxylate

By adding [4-(methyloxy)phenyl]magnesiumbromide 0.5M solution in THF (6 mL) and starting from 1,1-dimethylethyl 4-(2,2-dimethyl-4,6-dioxo-1,3-dioxan-5-ylidene)-1-piperidinecarboxylate (500 mg), 770 mg of the title compound were obtained without any chromatographic purification.

MS (ES/−): m/z=432 [M−H]⁻.

### Intermediate 36

### 1,1-dimethylethyl 4-(2,3-dihydro-1-benzofuran-5-yl)-4-(2,2-dimethyl-4,6-dioxo-1,3-dioxan-5-yl)-1-piperidinecarboxylate

Starting from 5-bromo-2,3-dihydro-1-benzofuran (2.98 g) and using 1,1-dimethylethyl 4-(2,2-dimethyl-4,6-dioxo-1,3-dioxan-5-ylidene)-1-piperidinecarboxylate (1 g), 2 g of the title compound were obtained without any chromatographic purification.

HPLC (walk-up): t_{R}=5.02 min.

### Intermediate 37

### 1,1-dimethylethyl 4-(1-benzofuran-5-yl)-4-(2,2-dimethyl-4,6-dioxo-1,3-dioxan-5-yl)-1-piperidinecarboxylate

Starting from 5-bromo-1-benzofuran (824 mg) and using 1,1-dimethylethyl 4-(2,2-dimethyl-4,6-dioxo-1,3-dioxan-5-ylidene)-1-piperidinecarboxylate (1 g), 940 mg of the title compound were obtained without any chromatographic purification.

HPLC (walk-up): t_{R}=5.023 min.

### Intermediate 38

### 1,1-dimethylethyl 4-(3-cyanophenyl)-4-(2,2-dimethyl-4,6-dioxo-1,3-dioxan-5-yl)-1-piperidinecarboxylate

A solution of 2-bromopropane 1.0M solution in THF (4.7 mL) in anhydrous THF (30 mL) was dropped into a suspension of magnesium turning (1.46 g) and in anhydrous THF (20 mL) under a Nitrogen atmosphere. The mixture refluxed for 45 min, then it was allowed to cool to rt and added drop-wise to a mixture of 3-iodobenzonitrile (2.11 g) in dry THF (20 mL) previously cooled at −40° C. under a nitrogen atmosphere. After stirring for 1 h under these conditions, a portion (6.5 mL) of the solution was dropped over 1,1-dimethylethyl 4-(2,2-dimethyl-4,6-dioxo-1,3-dioxan-5-ylidene)-1-piperidinecarboxylate (700 mg) and copper iodide (123 mg) in anhydrous THF (10 mL) previously cooled to 0° C. under a Nitrogen atmosphere. The mixture was allowed to warm to rt and stirred at 23° C. for 1.5 h. The mixture was treated with aqueous std NH4Cl solution and extracted with AcOEt. The combined organic extracts were collected, dried and concentrated under vacuum to give the title compound (1.9 g) as a white foam.

MS (ES/−): m/z=427 [M−H]⁻.

### Intermediate 39

### (4-(1,3-benzodioxol-5-yl)-1-{[(1,1-dimethylethyl)oxy]carbonyl}-4-piperidinyl)acetic Acid

A mixture of intermediate 32 (620 mg) in 3-pentanone (80 mL), and water (40 mL) was heated to 102° C. for 72 hours. The solution was allowed to cool to rt and the organic phase was separated. The aqueous phase was acidified to pH=3 and extracted with AcOEt (2×100 mL).

The combined organic phases were dried and concentrated in vacuo to give the crude which was purified by Biotage flash chromatography (elution with CH:AcOEt=9:1 to 1:1) to give the title compound (310 mg) as a yellow oil.

T.I.c.: CH:AcOEt=1:1, Rf=0.25 (detection with ninhydrine).

MS (ES/−): m/z=362 [M−H]⁻.

Following the same procedure described for intermediate 39, intermediates 40 and 41 were obtained.

### Intermediate 40

### {1-{[(1,1-dimethylethyl)oxy]carbonyl}-4-[3-fluoro-4-(methyloxy)phenyl]-4-piperidinyl}acetic Acid

Starting from intermediate 33 (800 mg), 620 mg of the title compound were obtained.

T.I.c.: CH:AcOEt=1:1, Rf=0.13 (detection with ninhydrine).

MS (ES/−): m/z=366 [M−H]⁻.

### Intermediate 41

### [1-{[(1,1-dimethylethyl)oxy]carbonyl}-4-(3-fluoro-4-methylphenyl)-4-piperidinyl]acetic Acid

Starting from intermediate 34 (3.12 g), 1.78 g of the title compound were obtained.

T.I.c.: CH:AcOEt 1:1, Rf=0.13 (detection with ninhydrine).

NMR (CDCl₃): δ (ppm) 7.16 (t, 1H); 7.02-6.96 (m, 2H); 3.67 (bd, 2H); 3.17 (bt, 2H); 2.59 (s, 2H); 2.27 (s, 3H); 2.23 (bd, 2H); 1.91 (t, 2H); 1.46 (s, 9H).

### Intermediate 42

### {1′-{[(1,1-dimethylethyl)oxy]carbonyl}-4-[4-(methyloxy)phenyl]-4-piperidinyl}acetic Acid

A mixture of intermediate 35 (600 mg) in 3-pentanone (6 mL), and water (2 mL) was processed by microwave irradiation at 150° C. for 12 min (2 cycles). The solution was allowed to cool to rt, the organic phase was separated and evaporated under vacuum. The crude was then dissolved in a mixture of CH:Et₂O=1:1 and aqueous 1.0 M NaOH was added; the aqueous phase was separated and washed again with CH:Et₂O=1:1. Then it was acidified to pH=5 and extracted with AcOEt. The organic phase was dried and concentrated in vacuo to give the title compound (262 mg) as a yellow brown oil.

MS (ES/−): m/z=348 [M−H]⁻.

Following the same procedure described for intermediate 42, intermediates 43, 44, 45 were obtained.

### Intermediate 43

### (4-(2,3-dihydro-1-benzofuran-5-yl)-1-{[(1,1-dimethylethyl)oxy]carbonyl}-4-piperidinyl)acetic Acid

Starting from intermediate 36 (2 g), 630 mg of the title compound were obtained as a yellow foam.

NMR (CDCl₃): δ (ppm) 7.17 (d, 1H); 7.06 (dd, 1H); 6.75 (d, 1H); 4.57 (m, 2H); 3.67 (bd, 2H); 3.21 (m, 2H); 3.16 (bd, 2H); 2.57 (s, 2H); 2.25 (bd, 2H); 1.89 (m, 2H); 1.46 (s, 9H).

### Intermediate 44

### (4-(1-benzofuran-5-yl)-1-[([(1,1-dimethylethyl)oxy]carbonyl]-4-piperidinyl)acetic Acid

Starting from intermediate 37 (940 mg), 170 mg of the title compound were obtained as a yellow-brown foam.

MS (ES/−): m/z=358 [M−H]⁻.

### Intermediate 45

### (4-(3-cyanophenyl)-1-{[(1,1-dimethylethyl)oxy]carbonyl}-4-piperidinyl)acetic Acid

Starting from intermediate 38 (1.9 mg), 263 mg of the title compound were obtained as a yellow oil.

NMR (CDCl₃): δ (ppm) 7.64 (d, 1H); 7.62 (td, 1H); 7.57 (td, 1H); 7.5 (t, 1H); 3.64 (bm, 2H); 3.26 (tm, 2H); 2.67 (s, 2H); 2.24 (dm, 2H); 1.99 (tm, 2H); 1.47 (s, 9H).

### Intermediate 46

### Phenylmethyl 2-ethenyl-4-oxo-3,4-dihydro-1(2H)-pyridinecarboxylate

4-(methyloxy)pyridine (4.52 g) was dissolved in dry THF (100 mL) at rt under a nitrogen atmosphere; and a solution of phenylmethyl chloridocarbonate (6.4 mL) in dry THF (75 mL) was added dropwise. Then the mixture was cooled at −78° C., and vinyl magnesium bromide 1.0M solution in THF (50 mL) was added. After stirring for 2 h aqueous 10% HCl was added and the mixture was allowed to warm to rt. The mixture was stirred for 1 h and then the organic phase was separated, washed with aqueous std NaHCO₃, with brine, dried and concentrated under vacuum to give the crude which was purified by Biotage flash cromathography (elution with CH:AcOEt=75:25) to afford the title compound (8.7 g) as pale yellow oil.

MS (ES/+): m/z=258 [M+H]⁺.

### Intermediate 47

### 1,1-dimethylethyl 2-ethenyl-4-oxo-3,4-dihydro-1 (2H)-pyridinecarboxylate

To a solution of intermediate 46 (5.3 g) in MeOH (100 mL), at 0° C. and under a nitrogen atmosphere, sodium methoxide (1.71 g) was added and the mixture was allowed to warm to rt. After stirring for 1 h, MeOH was removed by evaporation and the residue was dissolved in CH₃CN (100 mL); di-t-butyl-dicarbonate (7.0 g) and dimethylaminopyridine (4.18 g) were added. The mixture was stirred for 1 h and then CH₃CN was removed by evaporation and AcOEt (400 mL) was added, washed with water, with brine, dried and concentrated under vacuum to give the crude which was purified by Biotage flash chromatography (elution with CH:AcOEt=8:2) to afford the title compound (4.3 g) as a yellow oil.

T.I.c.: CH/AcOEt 7:3, Rf=0.41 (detection with ninhydrine).

MS (ES/+): m/z=168 [M-t-but+H]⁺.

### Intermediate 48

### 1,1-dimethylethyl 2-ethenyl-4-oxo-1-piperidinecarboxylate

To a solution of intermediate 47 (400 mg) in dry THF (8 mL), cooled at −78° C. and under a nitrogen atmosphere, L-selecride 1.0M solution in THF (2.7 mL) was added. After stirring under these conditions for 20 min, water (20 mL) and brine were added (10 mL) and the aqueous phase was extracted with AcOEt (3×50 mL). The organic phase was then dried and concentrated under vacuum to give the crude which was purified by flash chromatography (elution with CH:AcOEt=7:3) to afford the title compound (273 mg) as a yellow oil.

T.I.c.: CH/AcOEt 7:3, Rf=0.47 (detection with ninhydrine).

MS (ES/+): m/z=170 [M-t-but+H]⁺.

### Intermediate 49

### 1,1-dimethylethyl 4-(2,2-dimethyl-4,6-dioxo-1,3-dioxan-5-ylidene)-2-methyl-1-piperidinecarboxylate

To a solution of 2,2-dimethyl-1,3-dioxane-4,6-dione (Meldrum's acid) (12.8 g) in MeOH (190 mL), ammonium acetate (1.4 g) and 1,1-dimethylethyl 2-methyl-4-oxo-1-piperidinecarboxylate (19 g) were added. The solution was stirred at rt for 36 h, then MeOH was removed by evaporation under vacuum to afford the title compound (29.6 g) as a pale yellow solid.

T.I.c.: CH:AcOEt=7:3, Rf=0.22 (detection with ninhydrine).

MS (ES/+): m/z=362 [M+Na]⁺.

MS (ES/−): m/z=338 [M−H]⁻.

### Intermediate 50

### 1,1-dimethylethyl 4-(2,2-dimethyl-4,6-dioxo-1,3-dioxan-5-ylidene)-2-ethenyl-1-piperidinecarboxylate

A round bottom flask was charged with 1,1-dimethylethyl 2-ethenyl-4-oxo-1-piperidinecarboxylate (1.22 g), 2,2-dimethyl-1,3-dioxane-4,6-dione (Meldrum's acid) (770 mg), ammonium acetate (74 mg) in anhydrous toluene (3 mL). The mixture was stirred for 18 h at rt then the organic solution was dried and concentrated under vacuum to afford the title compound (1.68 g) as a yellow solid.

MS (ES/−): m/z=350 [M−H]⁻.

HPLC (walk-up): t_{R}=5.48 min.

### Intermediate 51 and 52

### 1,1-dimethylethyl 4-(2,2-dimethyl-4,6-dioxo-1,3-dioxan-5-yl)-4-(4-fluorophenyl)-2-methyl-1-piperidinecarboxylate (Intermediate 51—Anti Isomer)

### 1,1-dimethylethyl 4-(2,2-dimethyl-4,6-dioxo-1,3-dioxan-5-yl)-4-(4-fluorophenyl)-2-methyl-1-piperidinecarboxylate (Intermediate 52—Syn Isomer)

A 4-fluorophenyl magnesium bromide 1M solution in THF (6.5 mL) was added drop-wise to a mixture of intermediate 49 (4.5 g) and copper iodide (750 mg) in anhydrous THF (45 mL) previously cooled to 0° C. under a Nitrogen atmosphere. The mixture was stirred under these conditions for 15 min and then allowed to warm to rt and stirred at 23° C. for 1 h. The mixture was cooled to 0° C., treated with aqueous std NH₄Cl (40 mL) and aqueous NH₄OH (10 mL); THF was removed by evaporation and the organic phase was extracted with AcOEt (3×60 mL). The combined organic extracts were collected, dried over Na₂SO₄ and concentrated under vacuum. The residue was purified by flash chromatography (CH/AcOEt 9:1 to 7:3) to give the title compounds 51 (925 mg) as a yellow oil and 52 (1.89 g) as a yellow solid which were characterized as follows:

### Intermediate 51:

HPLC (walk-up): t_{R}=6.08 min.

NMR (CDCl₃): δ (ppm) 7.24 (dd, 2H); 6.99 (t, 2H); 3.91 (m, 1H); 3.85 (m, 1H); 3.65 (s, 1H); 3.2 (m, 1H); 2.25-2.50 (m, 4H); 1.55 (s, 3H); 1.29 (s, 9H); 1.23 (d, 3H); 1.01 (s, 3H). MS (ES/−): m/z=434 [M−H]⁻.

### Intermediate 52:

HPLC (walk-up): t_{R}=6.23 min.

NMR (CDCl₃): δ (ppm) 7.33 (dd, 2H); 7.06 (t, 2H); 4.42 (m, 1H); 4.01 (dt, 1H); 3.25 (s, 1H); 2.96 (dm, 1H); 2.88 (t, 1H): 2.79 (dt, 1H); 2.25 (dd, 1H); 1.95 (td, 1H); 1.53 (s, 3H); 1.44 (s, 9H); 0.97 (s, 3H); 0.63 (d, 3H).

MS (ES/−): m/z=434 [M−H]⁻.

Following the same procedure described for intermediate 51 and 52, intermediates 53, 54 and 55, 56 were obtained.

### Intermediate 53 and 54

### 1,1-dimethylethyl 4-(2,2-dimethyl-4,6-dioxo-1,3-dioxan-5-yl)-2-methyl-4-phenyl-1-piperidinecarboxylate (Intermediate 53—Anti Isomer)

### 1,1-dimethylethyl 4-(2,2-dimethyl-4,6-dioxo-1,3-dioxan-5-yl)-2-methyl-4-phenyl-1-piperidinecarboxylate (Intermediate 54—Syn Isomer)

Starting from phenylmagnesium bromide 1.0M solution in THF (26.5 mL) and using intermediate 49 (4.5 g), 610 mg of the title compound 53 and 495 mg of the title compound 54 were obtained characterized as follows:

### Intermediate 53:

HPLC (walk-up): t_{R}=6.05 min.

NMR (CDCl₃): δ (ppm) 7.20-7.32 (m, 5H); 3.88 (m, 2H); 3.67 (s, 1H); 3.23 (m, 1H); 2.30-2.50 (m, 4H); 1.52 (s, 3H); 1.28 (s, 9H); 1.25 (d, 3H); 0.85 (s, 3H).

MS (ES/−): m/z=416 [M−H]⁻.

### Intermediate 54:

HPLC (walk-up): t_{R}=6.18 min.

NMR (CDCl₃): δ (ppm) 7.20-7.32 (m, 5H); 4.38 (m, 1H); 3.97 (dt, 1H); 3.2 (s, 1H); 2.95 (dm, 1H); 2.87 (t, 1H); 2.8 (dt, 1H); 2.21 (dd, 1H); 1.91 (td, 1H); 1.46 (s, 3H); 1.4 (s, 9H); 0.77 (s, 3H); 0.58 (d, 3H).

MS (ES/−): m/z=416 [M−H]⁻.

### Intermediate 55 and 56

### 1,1-dimethylethyl 4-(2,2-dimethyl-4,6-dioxo-1,3-dioxan-5-yl)-2-ethenyl-4-(4-fluorophenyl)-1-piperidinecarboxylate (Intermediate 55—Anti Isomer)

### 1,1-dimethylethyl 4-(2,2-dimethyl-4,6-dioxo-1,3-dioxan-5-yl)-2-ethenyl-4-(4-fluorophenyl)-1-piperidinecarboxylate (Intermediate 56—Syn Isomer)

Starting from 4-fluoro-phenylmagnesium bromide 1.0M solution in THF (2.2 mL) and using intermediate 50 (260 mg), 60 mg of the title compound 55 and 8 mg of the title compound 56 were obtained characterized as follows:

### Intermediate 55:

NMR (CDCl₃): δ (ppm) 7.27 (dd, 2H); 6.98 (t, 2H); 6.14 (ddd, 1H); 5.17 (dd, 1H); 5.04 (dt, 1H); 4.9 (m, 1H); 4.21 (s, 1H); 4.15 (dt, 1H); 3.32 (dt, 1H); 3.24 (tt, 1H); 2.88 (dq, 1H); 1.94 (dd, 1H); 1.74 (td, 1H); 1.52 (s, 3H); 1.41 (s, 9H); 0.94 (s, 3H).

MS (ES/−): m/z=446 [M−H]⁻.

### Intermediate 56:

NMR (CDCl₃): δ (ppm) 7.27 (dd, 2H); 6.98 (t, 2H); 5.14 (ddd, 1H); 4.8 (bm, 2H); 4.68 (dt, 2H); 4.06 (m, 1H); 3.25 (s, 1H); 2.97 (m, 3H); 2.31 (dt, 1H); 1.94 (ft, 1H); 1.52 (s, 3H); 1.41 (s, 9H); 0.95 (s, 3H).

MS (ES/−): m/z=446 [M−H]⁻.

### Intermediate 57 and 58

### 1,1-dimethylethyl 4-(2,2-dimethyl-4,6-dioxo-1,3-dioxan-5-yl)-2-methyl-4-[4-(methyloxy)phenyl]-1-piperidinecarboxylate (Intermediate 57—Anti Isomer)

### 1,1-dimethylethyl 4-(2,2-dimethyl-4,6-dioxo-1,3-dioxan-5-yl)-2-methyl-4-[4-(methyloxy)phenyl]-1-piperidinecarboxylate (Intermediate 58—Syn Isomer)

A solution of 1-bromo-4-(methyloxy)benzene (4.4 mL) in anhydrous THF (30 mL) was slowly dropped into a suspension of magnesium turnings (1.16 mg) in dry THF (10 mL) under a Nitrogen atmosphere. The mixture refluxed for 30 minutes, then it was allowed to cool to rt and added drop-wise to a mixture of intermediate 49 (4.5 g) and copper iodide (757 mg) in anhydrous THF (40 mL) previously cooled to 0° C. under a Nitrogen atmosphere. The mixture was allowed to warm to rt and stirred at 23° C. for 2 h. The mixture was treated with aqueous std NH₄Cl and extracted with AcOEt The combined organic extracts were collected, dried and concentrated under vacuum. The residue was purified by flash chromatography (CH:AcOEt 9:1 to 6:4) to give the title compounds 57 (1 g) and 58 (1.5 g) characterized as follows:

### Intermediate 57:

T.I.c.: CH/AcOEt 7:3, Rf-0.23 (detection with ninhydrin).

NMR (CDCl₃): δ (ppm) 7.2 (d, 2H); 6.9 (d, 2H); 3.94-3.9 (m, 2H); 3.79 (s, 3H); 3.68 (s, 1H); 3.28 (m, 1H); 2.46 (m, 2H); 2.39 (m, 2H); 1.58 (s, 3H); 1.34 (m, 12H); 0.95 (s, 3H).

MS (ES/−): m/z=446 [M−H]⁻.

### Intermediate 58:

T.I.c.: CH/AcOEt 7:3, Rf=0.31 (detection with ninhydrin).

NMR (CDCl₃): δ (ppm) 7.25 (d, 2H); 6.9 (d, 2H); 4.45 (t, 1H); 4.0 (m, 1H); 3.82 (s, 3H); 3.23 (s, 1H); 2.95 (m, 1H); 2.9 (m, 1H); 2.83 (m, 1H); 2.1 (m, 1H); 1.95 (m, 1H); 1.53 (s, 3H); 1.45 (s, 9H); 0.91 (s, 3H); 0.67 (d, 3H).

MS (ES/−): m/z=446 [M−H]⁻.

Following the same procedure described for intermediate 57 and 58, intermediates 59, and 60 were obtained.

### Intermediate 59 and 60

### 1,1-dimethylethyl 4-(2,3-dihydro-1-benzofuran-5-yl)-4-(2,2-dimethyl-4,6-dioxo-1,3-dioxan-5-yl)-2-methyl-1-piperidinecarboxylate (Intermediate 59—Anti Isomer)

### 1,1-dimethylethyl 4-(2,3-dihydro-1-benzofuran-5-yl)-4-(2,2-dimethyl-4,6-dioxo-1,3-dioxan-5-yl)-2-methyl-1-piperidinecarboxylate (Intermediate 60—Syn Isomer)

Starting from 5-bromo-2,3-dihydro-1-benzofuran (7 g) and using intermediate 49 (4.5 g) 710 mg of the title compound 59 and 530 mg of the title compound 60 were obtained characterized as follows:

### Intermediate 59:

HPLC (walk-up): t_{R}=5.87 min.

NMR (CDCl₃): δ (ppm) 7.33 (dd, 1H); 7.07 (t, 1H); 7.0 (td, 1H); 4.48 (s, 1H); 3.99-3.81 (m, 4H); 2.72-2.53 (m, 4H); 2.46 (s, 3H); 1.86-1.74 (m, 2H); 1.36 (s, 9H); 0.94 (t, 3H).

### Intermediate 60:

HPLC (walk-up): t_{R}=6.02 min.

NMR (CDCl₃): δ (ppm) 7.33 (dd, 1H); 7.07 (t, 1H); 7.0 (td, 1H); 4.48 (s, 1H); 3.99-3.81 (m, 4H); 2.72-2.53 (m, 4H); 2.46 (s, 3H); 1.86-1.74 (m, 2H); 1.36 (s, 9H); 0.94 (t, 3H).

### Intermediate 61

### [1-{[(1,1-dimethylethyl)oxy]carbonyl}-4-(4-fluorophenyl)-2-methyl-4-piperidinyl]acetic Acid (Syn Isomer)

A mixture of intermediate 52 (1.89 g) in 3-pentanone (12 mL), and water (4 mL) was processed by microwave irradiation at 140° C. (2 cycles of 12 min and one cycle of 10 min). The solution was allowed to cool to rt, the organic phase was separated and evaporated under vacuum.

The crude was then dissolved in a mixture of CH:Et₂O=1:1 (30 mL) and aqueous 1.0 M NaOH (30 mL) was added; then it was acidified to pH=5 and extracted with AcOEt (3×20 mL). The organic phase was dried and concentrated in vacuo to give the title compound (875 mg) as a yellow solid.

T.I.c.: CH/AcOEt=1:1, Rf=0.15 (detection with ninhydrine).

MS (ES/−): m/z=350 [M−H]⁻.

NMR (CDCl₃): δ (ppm) 7.35 (dd, 2H); 7.03 (t, 2H); 4.32 (m, 1H); 3.99 (dt, 1H); 3.06 (td, 1H); 2.58 (d, 1H); 2.42 (d, 1H); 2.28 (dt, 1H); 2.07 (m, 2H); 1.77 (tm, 1H); 1.47 (s, 9H); 0.67 (d, 3H).

Following the same procedure described for intermediate 61, intermediates 62, 63, 64, 65 were obtained.

### Intermediate 62

### (1-{[(1-dimethylethyl)oxy]carbonyl}-2-methyl-4-phenyl-4-piperidinyl)acetic Acid (Syn Isomer)

Starting from intermediate 54 (495 mg), 380 mg of the title compound were obtained as a white foam by chromatographic purification eluting by AcOEt: CH=7:3.

NMR (CDCl₃): δ (ppm) 7.36 (dd, 2H); 7.31 (t, 2H); 7.22 (m, 1H); 4.3 (m, 1H); 3.97 (dt, 1H); 3.08 (td, 1H); 2.67 (dm, 1H); 2.59 (d, 1H); 2.42 (d, 1H); 2.32 (dt, 1H); 2.04 (dd, 1H); 1.76 (td, 1H); 1.44 (s, 9H); 0.64 (d, 3H).

### Intermediate 63

### [1-{[(1,1-dimethylethyl)oxy]carbonyl}-2-ethenyl-4-(4-fluorophenyl)-4-piperidinyl]acetic Acid (Syn Isomer)

Starting from intermediate 56 (54 mg), 30 mg of the title compound were obtained as a yellow foam by chromatographic purification eluting by AcOEt CH=7:3.

NMR (CDCl₃): δ (ppm) 7.26 (dd, 2H); 6.96 (t, 2H); 5.14 (m, 1H); 4.52-4.55 (m, 2H); 3.99 (bt, 1H); 3.08 (td, 1H); 2.63 (dd, 1H); 2.56 (d, 1H); 2.45 (bd, 1H); 2.41 (d, 1H); 2.08 (dd, 1H); 1.77 (td, 1H); 1.46 (dt, 1H); 1.43 (s, 9H).

### Intermediate 64

### {1-{[(1,1-dimethylethyl)oxy]carbonyl}-2-methyl-4-[4-(methyloxy)phenyl]-4-piperidinyl}acetic Acid (Syn Isomer)

Starting from intermediate 58 (1.5 g), 500 mg of the title compound were obtained as a yellow foam.

MS (ES/−): m/z=362 [M−H]⁻.

NMR (CDCl₃): δ (ppm) 7.25 (d, 2H); 6.9 (d, 2H); 4.45 (t, 1H); 4.03 (m, 1H); 3.1 (m, 1H); 3.82 (s, 3H); 2.7 (m, 1H); 2.3 (m, 1H); 2.59-2.39 (dd, 2H); 2.1 (m, 1H); 1.75 (m, 1H); 1.45 (s, 9H); 0.67 (d, 3H).

### Intermediate 65

### (4-(2,3-dihydro-1-benzofuran-5-yl)-1 {[(1,1-dimethylethyl)oxy]carbonyl}-2-methyl-4-piperidinyl)acetic Acid (Syn Isomer)

Starting from intermediate 60 (530 mg), 220 mg of the title compound were obtained as a yellow solid.

MS (ES/−): m/z=374 [M−H]⁻.

NMR (CDCl₃): δ (ppm) 7.24 (d, 1H); 7.06 (d, 1H); 6.7 (d, 1H); 4.54 (t, 2H); 4.25 (bm, 1H); 3.93 (m, 1H); 3.17 (t, 2H); 3.05 (m, 1H); 2.53 (d, 1H); 2.37 (d, 1H); 2.6 (m, 1H); 2.21 (m, 1H); 2.00 (m, 1H); 1.71 (m, 1H); 1.41 (s, 9H); 0.67 (d, 3H).

### Intermediate 66

### 1,1-Dimethylethyl 4-{2-[[(3-chloro-1-naphthalenyl)methyl](methyl)amino]-2-oxoethyl}-4-(4-fluorophenyl)-1-piperidinecarboxylate

A solution of [1-{[(1,1-dimethylethyl)oxy]carbonyl}-4-(4-fluorophenyl)-4-piperidinyl]acetic acid (100 mg), O-(7-Azabenzotriazol-1-yl)-N,N,N′,N′-tetramethyluronium hexafluorophosphate (146 mg) and TEA (0.123 ml) in anhydrous DCM (5 ml) was stirred at rt for 1 h under a Nitrogen atmosphere. Intermediate 28 (67 mg) was added and the mixture stirred at rt overnight. The mixture was washed with aqueous 5% NaHCO₃, the organic layer was dried, concentrated in vacuo and the residue purified by flash chromatography (CH/AcOEt 2:8) to give the title compound (140 mg) as a white foam.

T.I.c.: CH/AcOEt 3:7, Rf=0.26 (detection with ninhydrine).

MS (ES/+): m/z=547 [M+Na]⁺.

Following the same procedure described for intermediate 66, intermediates 67, 68, 69, 70, 71, 72, 73, 74, 75, 76, 77 were obtained.

### Intermediate 67

### 1,1-Dimethylethyl 4-{2-[[(3-cyano-1-naphthalenyl)methyl](methyl)amino]-2-oxoethyl}-4-(4-fluorophenyl)-1-piperidinecarboxylate

Starting from [1-{[(1,1-dimethylethyl)oxy]carbonyl}-4-(4-fluorophenyl)-4-piperidinyl]acetic acid (61 mg) and intermediate 31 (39 mg), 140 mg of the title compound were obtained as a white solid.

T.I.c.: CH/AcOEt 6:4, Rf=0.21 (detection with ninhydrine).

NMR (CDCl₃): δ (ppm) 8.15 (s, 1H); 8.05 (d, 1H); 7.90 (d, 1H); 7.60 (m, 2H); 7.30-7.20 (m, 3H); 6.90 (t, 2H); 4.85 (s, 2H); 3.65 (d, 2H); 3.10 (t, 2H); 2.65 (s, 2H); 2.35-2.20 (s+d, 5H); 2.00 (t, 2H); 1.40 (s, 9H).

### Intermediate 68

### 1,1-Dimethylethyl 4-{2-[[(3-bromo-1-naphthalenyl)methyl](methyl)amino]-2-oxoethyl}-4-(4-fluorophenyl)-1-piperidinecarboxylate

Starting from [1-{[(1,1-dimethylethyl)oxy]carbonyl}-4-(4-fluorophenyl)-4-piperidinyl]acetic acid (61 mg) and intermediate 29 (35 mg), 58 mg of the title compound were obtained as a white solid.

T.I.c.: CH/AcOEt 7:3, Rf=0.12 (detection with ninhydrine).

NMR (CDCl₃): δ 11 (ppm) 7.90-7.25 (m, 8H); 6.80 (t, 2H)); 4.85 (s, 2H); 3.6 (m, 2H); 3.10 (m, 2H); 2.60 (s, 2H); 2.25 (s, 3H); 2.25-2.00 (m, 4H); 1.40 (s, 9H).

MS (ES/+): m/z=591, 593 [M+Na]⁺.

### Intermediate 69

### 1,1-dimethylethyl 4-[2-[[(5-bromo-1-benzofuran-7-yl)methyl](methyl)amino]-2-oxoethyl]-4-(4-fluorophenyl)-1-piperidinecarboxylate

Starting from [1-{[(1,1-dimethylethyl)oxy]carbonyl}-4-(4-fluorophenyl)-4-piperidinyl]acetic acid (100 mg) and intermediate 27 (90 mg), 164 mg of the title compound were obtained.

T.I.c.: CH/AcOEt 7:3, Rf=0.19 (detection with ninhydrine).

MS (ES/+): m/z=581, 583 [M+Na]⁺.

### Intermediate 70

### 1,1-dimethylethyl 4-{2-[[1-(5-bromo-1-benzofuran-7-yl)ethyl](methyl)amino]-2-oxoethyl}-4-(4-fluorophenyl)-1-piperidinecarboxylate

Starting from [1-{[(1,1-dimethylethyl)oxy]carbonyl}-4-(4-fluorophenyl)-4-piperidinyl]acetic acid (100 mg) and intermediate 26 (82 mg), 94 mg of the title compound were obtained as a yellow oil.

T.I.c.: CH/AcOEt 4:6, Rf=0.72 (detection with ninhydrine).

MS (ES/+): m/z=595-597 [M+Na]⁺.

### Intermediate 71

### 1,1-dimethylethyl 4-[2-[[(5-cyano-1-benzofuran-7-yl)methyl](methyl)amino]-2-oxoethyl]-4-(4-fluorophenyl)-1-piperidinecarboxylate

Starting from [1-{[(1,1-dimethylethyl)oxy]carbonyl}-4-(4-fluorophenyl)-4-piperidinyl]acetic acid (100 mg) and intermediate 21 (60 mg), 133 mg of the title compound were obtained as a yellow oil.

T.I.c.: CH/AcOEt 4:6, Rf=0.48 (detection with ninhydrine).

MS (ES/+): m/z=528 [M+Na]⁺.

### Intermediate 72

### 1,1-dimethylethyl 4-{2-[[1-(5-cyano-1-benzofuran-7-yl)ethyl](methyl)amino]-2-oxoethyl}-4-(4-fluorophenyl)-1-piperidinecarboxylate

Starting from [1-{[(1,1-dimethylethyl)oxy]carbonyl}-4-(4-fluorophenyl)-4-piperidinyl]acetic acid (146 mg) and intermediate 24 (95 mg), 204 mg of the title compound were obtained as a yellowish solid.

MS (ES/+): m/z=542 [M+Na]⁺.

### Intermediate 73

### 1,1-dimethylethyl 4-{2-[[1-(3-cyano-1-naphthalenyl)ethyl](methyl)amino]-2-oxoethyl}-4-(4-fluorophenyl)-1-piperidinecarboxylate (Enantiomer 2)

Starting from [1-{[(1,1-dimethylethyl)oxy]carbonyl}-4-(4-fluorophenyl)-4-piperidinyl]acetic acid (41 mg) and intermediate 13 (28 mg), 61 mg of the title compound were obtained as a white solid.

T.I.c: CH/AcOEt 6:4, Rf=0.3 (detection with ninhydrine).

MS (ES/+): m/z=552 [M+Na]⁺.

### Intermediate 74

### 1,1-dimethylethyl 4-{2-[[1-(3-cyano-1-naphthalenyl)ethyl](methyl)amino]-2-oxoethyl}-4-(4-fluoro-3-methylphenyl)-1-piperidinecarboxylate (Enantiomer 2)

Starting from intermediate [1-{[(1,1-dimethylethyl)oxy]carbonyl}-4-(4-fluoro-3-methylphenyl)-4-piperidinyl]acetic acid (43 mg) and intermediate 13 (28 mg), 65 mg of the title compound were obtained as a white solid.

T.I.c.: CH/AcOEt 6:4, Rf=0.3 (detection with ninhydrine).

MS (ES/+): m/z=566 [M+Na]⁺.

### Intermediate 75

### 1,1-dimethylethyl 4-[2-{[1-(3-cyano-1-naphthalenyl)ethyl](methyl)amino]-2-oxoethyl}-4-(4-fluorophenyl)-1-piperidinecarboxylate (Enantiomer 1)

Starting from intermediate [1-{[(1,1-dimethylethyl)oxy]carbonyl}-4-(4-fluorophenyl)-4-piperidinyl]acetic acid (40 mg) and intermediate 12 (29 mg), 55 mg of the title compound were obtained as a white solid.

T.I.c.: CH/AcOEt 1:1, Rf=0.4 (detection with ninhydrine).

MS (ES/+): m/z=552 [M+Na]⁺.

### Intermediate 76

### 1,1-dimethylethyl 4-{2-[[1-(3-cyano-1-naphthalenyl)ethyl](methyl)amino]-2-oxoethyl}-4-(4-fluoro-3-methylphenyl)-1-piperidinecarboxylate (Enantiomer 1)

Starting from intermediate [1-{[(1,1-dimethylethyl)oxy]carbonyl}-4-(4-fluoro-3-methylphenyl)-4-piperidinyl]acetic acid (40 mg) and intermediate 12 (30 mg), 42 mg of the title compound were obtained as a white solid.

T.I.c.: CH:AcOEt 1:1, Rf=0.6 (detection with ninhydrine).

MS (ES/+): m/z=566 [M+Na]⁺.

### Intermediate 77

### 1,1-dimethylethyl 4-[2-{[(3-cyano-6-fluoro-1-naphthalenyl)methyl](methyl)amino]-2-oxoethyl}-4-(4-fluorophenyl)-1-piperidinecarboxylate

Starting from intermediate [1-{[(1,1-dimethylethyl)oxy]carbonyl}-4-(4-fluorophenyl)-4-piperidinyl]acetic acid (63 mg) and intermediate 7 (40 mg), 100 mg of the title compound were obtained as a white foam.

T.I.c.: CH:AcOEt 1:1, Rf=0.39 (detection with ninhydrine).

NMR (CDCl₃): δ (ppm) 8.1 (m, 2H); 7.5 (dd, 1H); 7.4 (td, 1H); 7.25 (dd, 2H); 7.21 (s, 1H); 6.91 (t, 2H); 4.82 (s, 2H); 3.68 (m, 2H); 3.15 (t, 2H); 2.8 (s, 3H); 2.35 (s, 2H); 2.02 (m, 2H); 1.68 (m, 2H); 1.45 (s, 9H).

### Intermediate 78

### 1,1-dimethylethyl 4-(2-{[1-(3-chloro-1-naphthalenyl)ethyl]amino}-2-oxoethyl)-4-(4-fluorophenyl)-1-piperidinecarboxylate (Enantiomer 1)

DIPEA (300 μL) and O-(benzotriazol-1-yl)-N,N,N′N′-tetramethyluronium tetrafluoroborate (221 mg) were added to a solution of intermediate [1-{[(1,1-dimethylethyl)oxy]carbonyl}-4-(4-fluorophenyl)-4-piperidinyl]acetic acid (155 mg) in anhydrous DMF (4 mL) under a Nitrogen atmosphere. After stirring for 30 minutes, intermediate 16 (95 mg) was added. The mixture was stirred at rt for 2 days, then it was diluted with AcOEt, washed with aqueous std NaHCO₃, water and brine; then it was dried and evaporated under vacuum to give a crude which was purified by flash chromatography (CH/AcOEt from 9:1 to 8:2) to give the title compound (196 mg) as a colourless oil.

MS (ES/+): m/z=547 [M+Na]⁺.

Following the same procedure described for intermediate 78, intermediates from 79 to 115, were obtained.

### Intermediate 79

### 1,1-dimethylethyl 4-(2-{[1-(3-chloro-1-naphthalenyl)ethyl]amino}-2-oxoethyl)-4-(4-fluorophenyl)-1-piperidinecarboxylate (Enantiomer 2)

Starting from [1-{[(1,1-dimethylethyl)oxy]carbonyl}-4-(4-fluorophenyl)-4-piperidinyl]acetic acid (155 mg) and intermediate 15 (95 mg), 212 mg of the title compound were obtained as a colourless oil.

MS (ES/+): m/z=547 [M+Na]⁺.

### Intermediate 80

### 1,1-dimethylethyl 4-(2-{[1-(3-chloro-1-naphthalenyl)ethyl]amino}-2-oxoethyl)-4-(4-cyanophenyl)-1-piperidinecarboxylate (Enantiomer 1)

Starting from intermediate (4-(4-cyanophenyl)-1-{[(1,1-dimethylethyl)oxy]carbonyl}-4-piperidinyl)acetic acid (100 mg) and intermediate 16 (77 mg), 142 mg of the title compound were obtained as a colourless oil.

MS (ES/+): m/z=476 [M-t-but+H]⁺.

### Intermediate 81

### 1,1-dimethylethyl 4-{2-[[1-(3-chloro-1-naphthalenyl)ethyl](methyl)amino]-2-oxoethyl}-4-(4-cyanophenyl)-1-piperidinecarboxylate (Enantiomer 2)

Starting from intermediate (4-(4-cyanophenyl)-1-{[(1,1-dimethylethyl)oxy]carbonyl}-4-piperidinyl)acetic acid (48 mg) and intermediate 19 (46 mg), 31 mg of the title compound were obtained as a brown oil.

HPLC (walk-up): t_{R}=6.58 min.

### Intermediate 82

### 1,1-dimethylethyl 4-(1,3-benzodioxol-5-yl)-4-[2-[[1-(3,5-dichlorophenyl)ethyl](methyl)amino]-2-oxoethyl]-1-piperidinecarboxylate (Enantiomer 1)

Starting from intermediate 39 (80 mg) and [1-(3,5-dichlorophenyl)ethyl]methylamine (50 mg), 107 mg of the title compound were obtained as a white foam.

MS (ES/+): m/z=572 [M+Na]⁺.

T.I.c.: CH/AcOEt 6:4, Rf=0.33.

### Intermediate 83

### 1,1-dimethylethyl 4-(1,3-benzodioxol-5-yl)-4-{2-[[1-(3,5-dibromophenyl)ethyl](methyl)amino]-2-oxoethyl}-1-piperidinecarboxylate (Enantiomer 1)

Starting from intermediate 39 (80 mg) and [1-(3,5-dibromophenyl)ethyl]methylamine (71 mg), 128 mg of the title compound were obtained as a white foam.

MS (ES/+): m/z=661 [M+Na]⁺.

T.I.c.: CH/AcOEt 7:3, Rf=0.3.

### Intermediate 84

### 1,1-dimethylethyl 4-(2-{[1-(3-chloro-1-naphthalenyl)ethyl]amino}-2-oxoethyl)-4-(3-fluoro-4-methylphenyl)-1-piperidinecarboxylate (Enantiomer 1)

Starting from intermediate 41 (100 mg) and intermediate 16 (58 mg), 116 mg of the title compound were obtained as a white foam.

MS (ES/+): m/z=483 [M-t-but+H]⁺.

### Intermediate 85

### 1,1-dimethylethyl 4-(2-{[1-(3-chloro-1-naphthalenyl)ethyl]amino}-2-oxoethyl)-4-(3-fluoro-4-methylphenyl)-1-piperidinecarboxylate (Enantiomer 2)

Starting from intermediate 41 (100 mg) and intermediate 15 (58 mg), 105 mg of the title compound were obtained as a white foam.

MS (ES/+): m/z=483 [M-t-but+H]⁺.

### Intermediate 86

### 1,1-dimethylethyl 4-(2-{[1-(3-chloro-1-naphthalenyl)ethyl]amino}-2-oxoethyl)-4-(3-cyanophenyl)-1-piperidinecarboxylate (Enantiomer 1)

Starting from intermediate 45 (113 mg) and intermediate 16 (68 mg), 126 mg of the title compound were obtained as a white foam.

MS (ES/+): m/z=554 [M+Na]⁺.

### Intermediate 87

### 1,1-dimethylethyl 4-(2-{[1-(3-chloro-1-naphthalenyl)ethyl]amino}-2-oxoethyl)-4-(3-cyanophenyl)-1-piperidinecarboxylate (Enantiomer 2)

Starting from intermediate 45 (60 mg) and intermediate 15 (35 mg), 97 mg of the title compound were obtained as a white foam.

MS (ES/+): m/z=554 [M+Na]⁺.

### Intermediate 88

### 1,1-dimethylethyl 4-(2-{[1-(3-chloro-1-naphthalenyl)ethyl]amino}-2-oxoethyl)-4-phenyl-1-piperidinecarboxylate (Enantiomer 1)

Starting from (1-{[(1-methylethyl)oxy]carbonyl}-4-phenyl-4-piperidinyl)acetic acid (169 mg) and intermediate 16 (100 mg), 250 mg of the title compound were obtained as a white foam.

MS (ES/+): m/z=529 [M+Na]⁺.

### Intermediate 89

### 1,1-dimethylethyl 4-(2-{[1-(3-chloro-1-naphthalenyl)ethyl]amino}-2-oxoethyl)-4-phenyl-1-piperidinecarboxylate (Enantiomer 2)

Starting from (1-{[(1-methylethyl)oxy]carbonyl}-4-phenyl-4-piperidinyl)acetic acid (85 mg) and intermediate 15 (50 mg), 107 mg of the title compound were obtained as a white foam.

MS (ES/+): m/z=529 [M+Na]⁺.

### Intermediate 90

### 1,1-dimethylethyl 4-(1-benzofuran-5-yl)-4-(2-{[1-(3-chloro-1-naphthalenyl)ethyl]amino}-2-oxoethyl)-1-piperidinecarboxylate (Enantiomer 1)

Starting from intermediate 44 (112 mg) and intermediate 16 (70 mg), 122 mg of the title compound were obtained as a white foam.

MS (ES/+): m/z=491 [M-t-but+H]⁺.

### Intermediate 91

### 1,1-dimethylethyl 4-(1-benzofuran-5-yl)-4-(2-{[1-(3-chloro-1-naphthalenyl)ethyl]amino}-2-oxoethyl)-1-piperidinecarboxylate (Enantiomer 2)

Starting from intermediate 44 (50 mg) and intermediate 15 (31 mg), 55 mg of the title compound were obtained as a white foam.

MS (ES/+): m/z=491 [M-t-but+H]⁺.

### Intermediate 92

### 1,1-dimethylethyl 4-{2-[[1-(3-cyano-1-naphthalenyl)ethyl](methyl)amino]-2-oxoethyl}-4-[3-fluoro-4-(methyloxy)phenyl]-1-piperidinecarboxylate (Enantiomer 1)

Starting from intermediate 40 (182 mg) and intermediate 12 (104 mg), 276 mg of the title compound were obtained as a white foam without any chromatographic purification.

T.I.c.: CH/AcOEt 1:1, Rf=0.45 (detection with ninhydrine).

MS (ES/+): m/z=582 [M+Na]⁺.

### Intermediate 93

### 1,1-dimethylethyl 4-{2-[[1-(3-cyano-1-naphthalenyl)ethyl](methyl)amino]-2-oxoethyl}-4-[3-fluoro-4-(methyloxy)phenyl]-1-piperidinecarboxylate (Enantiomer 2)

Starting from intermediate 40 (166 mg) and intermediate 13 (95 mg), 300 mg of the title compound were obtained as a white foam without any chromatographic purification.

T.I.c.: CH/AcOEt 1:1, Rf=0.45 (detection with ninhydrine).

MS (ES/+): m/z=582 [M+Na]⁺.

### Intermediate 94

### 1,1-dimethylethyl 4-{2-[[(3-cyano-1-naphthalenyl)methyl](methyl)amino]-2-oxoethyl}-4-[3-fluoro-4-(methyloxy)phenyl]-1-piperidinecarboxylate

Starting from intermediate 40 (166 mg) and intermediate 31 (89 mg), 240 mg of the title compound were obtained as a white foam without any chromatographic purification.

T.I.c.: CH/AcOEt 1:1, Rf=0.28 (detection with ninhydrine).

MS (ES/+): m/z=568 [M+Na]⁺.

### Intermediate 95

### 1,1-dimethylethyl 4-(2-{[1-(3-chloro-1-naphthalenyl)ethyl]amino}-2-oxoethyl)-4-[4-(methyloxy)phenyl]-1-piperidinecarboxylate (Enantiomer 1)

Starting from intermediate 42 (120 mg) and intermediate 16 (63 mg), 166 mg of the title compound were obtained.

MS (ES/+): m/z=559 [M+Na]⁺.

### Intermediate 96

### 1,1-dimethylethyl 4-(2-{[1-(3-chloro-1-naphthalenyl)ethyl]amino}-2-oxoethyl)-4-[4-(methyloxy)phenyl]-1-piperidinecarboxylate (Enantiomer 2)

Starting from intermediate 42 (120 mg) and intermediate 15 (63 mg), 158 mg of the title compound were obtained.

MS (ES/+): m/z=481 [M-t-but+H]⁺.

### Intermediate 97

### 1,1-dimethylethyl 4-(2-{[1-(3-chloro-1-naphthalenyl)ethyl]amino}-2-oxoethyl)-4-(2,3-dihydro-1-benzofuran-5-yl)-1-piperidinecarboxylate (Enantiomer 1)

Starting from intermediate 43 (150 mg) and intermediate 16 (92 mg), 135 mg of the title compound were obtained.

MS (ES/+): m/z=493 [M-t-but+H]⁺.

### Intermediate 98

### 1,1-dimethylethyl 4-(2-{[1-(3-chloro-1-naphthalenyl)ethyl]amino}-2-oxoethyl)-4-(2,3-dihydro-1-benzofuran-5-yl)-1-piperidinecarboxylate (Enantiomer 2)

Starting from intermediate 43 (100 mg) and intermediate 15 (63 mg), 125 mg of the title compound were obtained.

MS (ES/+): m/z=571 [M+Na]⁺.

### Intermediate 99

### 1,1-dimethylethyl 4-{2-[[1-(3-cyano-1-naphthalenyl)ethyl](methyl)amino]-2-oxoethyl}-2-methyl-4-phenyl-1-piperidinecarboxylate (Syn Isomer, Chain Enantiomer 1)

Starting from intermediate 62 (50 mg) and intermediate 16 (34 mg), 45 mg of the title compound were obtained as a white foam.

T.I.c.: CH/AcOEt 7:3, Rf=0.27 (detection with ninhydrin).

MS (ES/+): m/z=470 [M-t-but+H]⁺.

### Intermediate 100

### 1,1-dimethylethyl 4-{2-[[1-(3-cyano-1-naphthalenyl)ethyl](methyl)amino]-2-oxoethyl}-2-methyl-4-phenyl-1-piperidinecarboxylate (Syn Isomer, Chain Enantiomer 2)

Starting from intermediate 62 (50 mg) and intermediate 15 (34 mg), 55 mg of the title compound were obtained as a white foam.

T.I.c.: CH/AcOEt 7:3, Rf=0.27 (detection with ninhydrin).

MS (ES/+): m/z=470 [M-t-but+H]⁺.

### Intermediate 101

### 1,1-dimethylethyl 4-{2-[[1-(3-cyano-1-naphthalenyl)ethyl](methyl)amino]-2-oxoethyl}-4-(4-fluorophenyl)-2-methyl-1-piperidinecarboxylate (Syn Isomer, Chain Enantiomer 1)

Starting from intermediate 61 (70 mg) and intermediate 16 (40 mg), 36 mg of the title compound were obtained as a white foam.

MS (ES/+): m/z=566 [M+Na]⁺.

### Intermediate 102

### 1,1-dimethylethyl 4-{2-[[1-(3-cyano-1-naphthalenyl)ethyl](methyl)amino]-2-oxoethyl}-4-(4-fluorophenyl)-2-methyl-1-piperidinecarboxylate (Syn Isomer, Chain Enantiomer 2)

Starting from intermediate 61 (70 mg) and intermediate 15 (40 mg), 74 mg of the title compound were obtained as a white foam.

MS (ES/+): m/z=566 [M+Na]⁺.

### Intermediate 103 and 104

### 1,1-dimethylethyl 4-(2-{[1-(3-chloro-1-naphthalenyl)ethyl]amino}-2-oxoethyl)-4-(4-fluorophenyl)-2-methyl-1-piperidinecarboxylate (Syn Isomer 1, Chain Enantiomer 1)

### 1,1-dimethylethyl 4-(2-{[1-(3-chloro-1-naphthalenyl)ethyl]amino}-2-oxoethyl)-4-(4-fluorophenyl)-2-methyl-1-piperidinecarboxylate (Syn Isomer 2, Chain Enantiomer 1)

Starting from intermediate 61 (130 mg) and intermediate 16 (76 mg), 92 mg of the title compound 103 and 65 mg of the title compound 104 were obtained as white foams.

### Intermediate 103:

T.I.c.: CH/AcOEt 6:4, Rf=0.35 (detection with ninhydrin).

MS (ES/+): m/z=561 [M+Na]⁺.

### Intermediate 104:

T.I.c.: CH/AcOEt 6:4, Rf=0.21 (detection with ninhydrin).

MS (ES/+): m/z=561 [M+Na]⁺.

### Intermediate 105 and 106

### 1,1-dimethylethyl 4-(2-{[1-(3-chloro-1-naphthalenyl)ethyl]amino}-2-oxoethyl)-4-(4-fluorophenyl)-2-methyl-1-piperidinecarboxylate (Syn Isomer 1, Chain Enantiomer 2)

### 1,1-dimethylethyl 4-(2-{[1-(3-chloro-1-naphthalenyl)ethyl]amino}-2-oxoethyl)-4-(4-fluorophenyl)-2-methyl-1-piperidinecarboxylate (Syn Isomer 2, Chain Enantiomer 2)

Starting from intermediate 61 (130 mg) and intermediate 15 (76 mg), 100 mg of the title compound 105 and 87 mg of the title compound 106 were obtained as white foams.

### Intermediate 105:

T.I.c.: CH/AcOEt 6:4, Rf=0.35 (detection with ninhydrin).

MS (ES/+): m/z=561 [M+Na]⁺.

### Intermediate 106:

T.I.c.: CH/AcOEt 6:4, Rf=0.21 (detection with ninhydrin).

MS (ES/+): m/z=561 [M+Na]⁺.

### Intermediate 107 and 108

### 1,1-dimethylethyl 4-(2-{[1-(3-chloro-1-naphthalenyl)ethyl]amino}-2-oxoethyl)-2-methyl-4-phenyl-1-piperidinecarboxylate (Syn Isomer 1, Chain Enantiomer 1)

### 1,1-dimethylethyl 4-(2-{[1-(3-chloro-1-naphthalenyl)ethyl]amino}-2-oxoethyl)-2-methyl-4-phenyl-1-piperidinecarboxylate (Syn Isomer 2, Chain Enantiomer 1)

Starting from intermediate 62 (90 mg) and intermediate 16 (61 mg), 48 mg of the title compound 107 and 38 mg of the title compound 108 were obtained as white foams.

### Intermediate 107:

HPLC (walk-up): t_{R}=7.15 min.

### Intermediate 108:

HPLC (walk-up): t_{R}=7.12 min.

### Intermediate 109 and 110

### 1,1-dimethylethyl 4-(2-{[1-(3-chloro-1-naphthalenyl)ethyl]amino}-2-oxoethyl)-2-methyl-4-phenyl-1-piperidinecarboxylate (Syn Isomer 1, Chain Enantiomer 2)

### 1,1-dimethylethyl 4-(2-{[1-(3-chloro-1-naphthalenyl)ethyl]amino}-2-oxoethyl)-2-methyl-4-phenyl-1-piperidinecarboxylate (Syn Isomer 2, Chain Enantiomer 2)

Starting from intermediate 62 (90 mg) and intermediate 15 (61 mg), 36 mg of the title compound 109 and 32 mg of the title compound 110 were obtained as white foams.

### Intermediate 109:

HPLC (walk-up): t_{R}=7.15 min.

### Intermediate 110:

HPLC (walk-up): t_{R}=7.12 min.

### Intermediate 111 and 112

### 1,1-dimethylethyl 4-(2-{[1-(3-chloro-1-naphthalenyl)ethyl]amino}-2-oxoethyl)-2-methyl-4-[4-(methyloxy)phenyl]-1-piperidinecarboxylate (Syn Isomer 1, Chain Enantiomer 1)

### 1,1-dimethylethyl 4-(2-{[1-(3-chloro-1-naphthalenyl)ethyl]amino}-2-oxoethyl)-2-methyl-4-[4-(methyloxy)phenyl]-1-piperidinecarboxylate (Syn Isomer 2, Chain Enantiomer 1)

Starting from intermediate 64 (90 mg) and intermediate 16 (51 mg), 54 mg of the title compound 111 and 66 mg of the title compound 112 were obtained as white foams.

### Intermediate 111:

MS (ES/+): m/z=495 [M-t-but+H]⁺.

T.I.c.: CH/AcOEt 1:1, Rf=0.33 (detection with ninhydrin).

### Intermediate 112:

MS (ES/+): m/z=495 [M-t-but+H]⁺.

T.I.c.: CH/AcOEt 1:1, Rf=0.28 (detection with ninhydrin).

### Intermediate 113 and 114

### 1,1-dimethylethyl 4-(2-{[1-(3-chloro-1-naphthalenyl)ethyl]amino}-2-oxoethyl)-4-(2,3-dihydro-1-benzofuran-5-yl)-2-methyl-1-piperidinecarboxylate (Syn Isomer 1, Chain Enantiomer 1)

### 1,1-dimethylethyl 4-(2-{[1-(3-chloro-1-naphthalenyl)ethyl]amino}-2-oxoethyl)-4-(2,3-dihydro-1-benzofuran-5-yl)-2-methyl-1-piperidinecarboxylate (Syn Isomer 2, Chain Enantiomer 1)

Starting from intermediate 65 (100 mg) and intermediate 16 (61 mg), 43 mg of the title compound 113 and 43 mg of the title compound 114 were obtained as white foams.

### Intermediate 113:

MS (ES/+): m/z=507 [M-t-but+H]⁺.

T.I.c.: CH/AcOEt 1:1, Rf=0.32 (detection with ninhydrin).

### Intermediate 114:

MS (ES/+): m/z=507 [M-t-but+H]⁺.

T.I.c.: CH/AcOEt 1:1, Rf=0.27 (detection with ninhydrin).

### Intermediate 115

### 1,1-dimethylethyl 4-(2-{[1-(3-chloro-1-naphthalenyl)ethyl]amino}-2-oxoethyl)-2-ethenyl-4-(4-fluorophenyl)-1-piperidinecarboxylate (Syn Isomer 1 Chain Enantiomer 1)

Starting from intermediate 63 (28 mg) and intermediate 16 (8 mg), 43 mg of the title compound were obtained as white foam.

MS (ES/+): m/z=495 [M-t-but+H]⁺.

HPLC (walk-up): t_{R}=7.14

### Intermediate 116

### 1,1-dimethylethyl 4-{2-[[1-(3-chloro-1-naphthalenyl)ethyl](methyl)amino]-2-oxoethyl}-4-(4-fluorophenyl)-1-piperidinecarboxylate (Enantiomer 1)

Intermediate 78 (196 mg) was dissolved in dry DMF (5 mL) and, under a Nitrogen atmosphere and at 0° C., NaH 60% dispersion in mineral oil (30 mg) was added. The mixture was allowed to warm to rt and stirred under these conditions for 20 min. Then methyl iodide was added (0.13 mL) and the solution was stirred for 2 h. Water and AcOEt were added; the organic phase separated and washed with brine, dried and evaporated under vacuum to give a crude which was purified by flash chromatography (elution with CH:AcOEt from 9:1 to 8:2) to afford the title compound (137 mg) as white foam.

MS (ES/+): m/z=561 [M+Na]⁺.

Following the same procedure described to obtain intermediate 116, intermediates from 117 to 141 were prepared.

### Intermediate 117

### 1,1-dimethylethyl 4-{2-[[1-(3-chloro-1-naphthalenyl)ethyl](methyl)amino]-2-oxoethyl}-4-(4-fluorophenyl)-1-piperidinecarboxylate (Enantiomer 2)

Starting from intermediate 79 (212 mg), 170 mg of the title compound were obtained as a white foam.

MS (ES/+): m/z=561 [M+Na]⁺.

### Intermediate 118

### 1,1-dimethylethyl 4-{2-[[1-(3-chloro-1-naphthalenyl)ethyl](methyl)amino]-2-oxoethyl}-4-(4-cyanophenyl)-1-piperidinecarboxylate

### (Enantiomer 1)

Starting from intermediate 80 (142 mg), 184 mg of the title compound were obtained as a yellow oil without any chromatographic purification.

MS (ES/+): m/z=568 [M+Na]⁺.

### Intermediate 119

### 1,1-dimethylethyl 4-{2-[[1-(3-chloro-1-naphthalenyl)ethyl](methyl)amino]-2-oxoethyl}-4-(3-fluoro-4-methylphenyl)-1-piperidinecarboxylate

### (Enantiomer 1)

Starting from intermediate 84 (116 mg), 93 mg of the title compound were obtained as a white foam.

MS (ES/+): m/z=575 [M+Na]⁺.

### Intermediate 120

### 1,1-dimethylethyl 4-{2-[[1-(3-chloro-1-naphthalenyl)ethyl](methyl)amino]-2-oxoethyl}-4-(3-fluoro-4-methylphenyl)-1-piperidinecarboxylate

### (Enantiomer 2)

Starting from intermediate 85 (105 mg), 91 mg of the title compound were obtained as a white foam.

MS (ES/+): m/z=575 [M+Na]⁺.

### Intermediate 121

### 1,1-dimethylethyl 4-{2-[[1-(3-chloro-1-naphthalenyl)ethyl](methyl)amino]-2-oxoethyl}-4-(3-cyanophenyl)-1-piperidinecarboxylate

### (Enantiomer 1)

Starting from intermediate 86 (126 mg), 80 mg of the title compound were obtained as a white foam.

MS (ES/+): m/z=568 [M+Na]⁺.

### Intermediate 122

### 1,1-dimethylethyl 4-{2-[[1-(3-chloro-1-naphthalenyl)ethyl](methyl)amino]-2-oxoethyl}-4-(3-cyanophenyl)-1-piperidinecarboxylate

### (Enantiomer 2)

Starting from intermediate 87 (97 mg), 33 mg of the title compound were obtained as a white foam.

MS (ES/+): m/z=568 [M+Na]⁺.

### Intermediate 123

### 1,1-dimethylethyl 4-{2-[[1-(3-chloro-1-naphthalenyl)ethyl](methyl)amino]-2-oxoethyl}-4-phenyl-1-piperidinecarboxylate

### (Enantiomer 1)

Starting from intermediate 88 (250 mg), 127 mg of the title compound were obtained as a white foam.

MS (ES/+): m/z=543 [M+Na]⁺.

### Intermediate 124

### 1,1-dimethylethyl 4-{2-[[1-(3-chloro-1-naphthalenyl)ethyl](methyl)amino]-2-oxoethyl}-4-phenyl-1-piperidinecarboxylate

### (Enantiomer 2)

Starting from intermediate 89 (107 mg), 71 mg of the title compound were obtained as a white foam.

MS (ES/+): m/z=543 [M+Na]⁺.

### Intermediate 125

### 1,1-dimethylethyl 4-(1-benzofuran-5-yl)-4-{2-[[1-(3-chloro-1-naphthalenyl)ethyl](methyl)amino]-2-oxoethyl}-1-piperidinecarboxylate

### (Enantiomer 1)

Starting from intermediate 90 (122 mg), 108 mg of the title compound were obtained as a white foam.

MS (ES/+): m/z=583 [M+Na]⁺.

### Intermediate 126

### 1,1-dimethylethyl 4-(1-benzofuran-5-yl)-4-{2-[[1-(3-chloro-1-naphthalenyl)ethyl](methyl)amino]-2-oxoethyl}-1-piperidinecarboxylate

### (Enantiomer 2)

Starting from intermediate 91 (55 mg), 51 mg of the title compound were obtained as a white foam.

MS (ES/+): m/z=583 [M+Na]⁺.

### Intermediate 127

### 1,1-dimethylethyl 4-{2-[[1-(3-chloro-1-naphthalenyl)ethyl](methyl)amino]-2-oxoethyl}-4-[4-(methyloxy)phenyl]-1-piperidinecarboxylate

### (Enantiomer 1)

Starting from intermediate 95 (166 mg), 47 mg of the title compound were obtained as a white foam.

MS (ES/+): m/z=573 [M+Na]⁺.

### Intermediate 128

### 1,1-dimethylethyl 4-{2-[[1-(3-chloro-1-naphthalenyl)ethyl](methyl)amino]-2-oxoethyl}-4-[4-(methyloxy)phenyl]-1-piperidinecarboxylate

### (Enantiomer 2)

Starting from intermediate 96 (158 mg), 57 mg of the title compound were obtained as a white foam.

MS (ES/+): m/z=573 [M+Na]⁺.

### Intermediate 129

### 1,1-dimethylethyl 4-{2-[[1-(3-chloro-1-naphthalenyl)ethyl](methyl)amino]-2-oxoethyl}-4-(2,3-dihydro-1-benzofuran-5-yl)-1-piperidinecarboxylate

### (Enantiomer 1)

Starting from intermediate 97 (135 mg), 88 mg of the title compound were obtained as a white foam.

MS (ES/+): m/z=507 [M-t-but+H]⁺.

### Intermediate 130

### 1,1-dimethylethyl 4-{2-[[1-(3-chloro-1-naphthalenyl)ethyl](methyl)amino]-2-oxoethyl}-4-(2,3-dihydro-1-benzofuran-5-yl)-1-piperidinecarboxylate

### (Enantiomer 2)

Starting from intermediate 98 (125 mg), 118 mg of the title compound were obtained as a white foam.

MS (ES/+): m/z=507 [M-t-but+H]⁺.

### Intermediate 131

### 1,1-dimethylethyl 4-[2-{[1-(3-chloro-1-naphthalenyl)ethyl](methyl)amino]-2-oxoethyl}-2-methyl-4-phenyl-1-piperidinecarboxylate (Syn Isomer 1, Chain Enantiomer 1)

Starting from intermediate 107 (46 mg), 47 mg of the title compound were obtained as a white foam without any chromatographic purification.

HPLC (walk-up): t_{R}=7.71 min.

### Intermediate 132

### 1,1-dimethylethyl 4-{2-[[1-(3-chloro-1-naphthalenyl)ethyl](methyl)amino]-2-oxoethyl}-2-methyl-4-phenyl-1-piperidinecarboxylate (Syn Isomer 2, Chain Enantiomer 1)

Starting from intermediate 108 (36 mg), 37 mg of the title compound were obtained as a white foam without any chromatographic purification.

HPLC (walk-up): t_{R}=7.68 min.

### Intermediate 133

### 1,1-dimethylethyl 4-{2-[[1-(3-chloro-1-naphthalenyl)ethyl](methyl)amino]-2-oxoethyl}-2-methyl-4-phenyl-1-piperidinecarboxylate (Syn Isomer 1, Chain Enantiomer 2)

Starting from intermediate 109 (48 mg), 42 mg of the title compound were obtained as a white foam without any chromatographic purification.

HPLC (walk-up): t_{R}=7.70 min.

### Intermediate 134

### 1,1-dimethylethyl 4-{2-[[1-(3-chloro-1-naphthalenyl)ethyl](methyl)amino]-2-oxoethyl}-2-methyl-4-phenyl-1-piperidinecarboxylate (Syn Isomer 2, Chain Enantiomer 2)

Starting from intermediate 110 (48 mg), 39 mg of the title compound were obtained as a white foam without any chromatographic purification.

HPLC (walk-up): t_{R}=7.68 min.

### Intermediate 135

### 1,1-dimethylethyl 4-{2-[[1-(3-chloro-1-naphthalenyl)ethyl](methyl)amino]-2-oxoethyl}-2-methyl-4-[4-(methyloxy)phenyl]-1-piperidinecarboxylate (Syn Isomer 1 Chain Enantiomer 1)

Starting from intermediate 111 (54 mg), 48 mg of the title compound were obtained as a white foam.

MS (ES/+): m/z=565 [M+H]⁺.

### Intermediate 136

### 1,1-dimethylethyl 4-{2-[[1-(3-chloro-1-naphthalenyl)ethyl](methyl)amino]-2-oxoethyl}-2-methyl-4-[4-(methyloxy)phenyl]-1-piperidinecarboxylate (Syn Isomer 2, Chain Enantiomer 1)

Starting from intermediate 112 (66 mg), 54 mg of the title compound were obtained as a white foam.

MS (ES/+): m/z=565 [M+H]⁺.

### Intermediate 137

### 1,1-dimethylethyl 4-{2-[[1-(3-chloro-1-naphthalenyl)ethyl](methyl)amino]-2-oxoethyl}-4-(2,3-dihydro-1-benzofuran-5-yl)-2-methyl-1-piperidinecarboxylate (Syn Isomer 1, Chain Enantiomer 1)

Starting from intermediate 113 (43 mg), 43 mg of the title compound were obtained as a white foam without any chromatographic purification.

MS (ES/+): m/z=599 [M+Na]⁺.

### Intermediate 138

### 1,1-dimethylethyl 4-{2-[[1-(3-chloro-1-naphthalenyl)ethyl](methyl)amino]-2-oxoethyl}-4-(2,3-dihydro-1-benzofuran-5-yl)-2-methyl-1-piperidinecarboxylate (Syn Isomer 2, Chain Enantiomer 1)

Starting from intermediate 114 (43 mg), 43 mg of the title compound were obtained as a white foam without any chromatographic purification.

MS (ES/+): m/z=599 [M+Na]⁺.

### Intermediate 139

### 1,1-dimethylethyl 4-{2-[[1-(3-chloro-1-naphthalenyl)ethyl](methyl)amino]-2-oxoethyl}-2-ethenyl-4-(4-fluorophenyl)-1-piperidinecarboxylate

### (Syn Isomer 1, Chain Enantiomer 1)

Starting from intermediate 115 (8.5 mg), 8.5 mg of the title compound were obtained as a white foam.

MS (ES/+): m/z=509 [M-t-but+H]⁺.

HPLC (walk-up) t_{R}=7.7

### Intermediate 140

### 1,1-dimethylethyl 4-{2-[[1-(3-chloro-1-naphthalenyl)ethyl](methyl)amino]-2-oxoethyl}-4-[3-fluoro-4-(methyloxy)phenyl]-1-piperidinecarboxylate

### (Enantiomer 1)

DIPEA (290 μL) and O-(benzotriazol-1-yl)-N,N,N′N′-tetramethyluronium tetrafluoroborate (239 mg) were added to a solution of intermediate 40 (249 mg) in anhydrous DMF (4 mL) under a Nitrogen atmosphere. After stirring for 30 minutes, intermediate 16 (140 mg) was added. The mixture was stirred at rt overnight, then it was diluted with AcOEt, washed with aqueous std NaHCO₃, water and brine; then it was dried and evaporated under vacuum to give a crude product [T.I.c.: CH/AcOEt 1:1, Rf=0.50 (detection with ninhydrin)]. This intermediate was dissolved in dry DMF (4 mL) and, under a Nitrogen atmosphere and at 0° C., NaH 60% dispersion in mineral oil (53 mg) was added. The mixture was allowed to warm to rt and stirred under these conditions for 20 min. Then methyl iodide was added (0.41 mL) and the solution was stirred for 2 h at 50° C. Water and AcOEt were added; the organic phase separated and washed with brine, dried and evaporated under vacuum to give the title compound (396 mg) as white foam without any further purification.

T.I.c.: CH/AcOEt 1:1, Rf=0.62 (detection with ninhydrin)

MS (ES/+): m/z=591 [M+Na]⁺.

### Intermediate 141

### 1,1-dimethylethyl 4-{2-[[1-(3-chloro-1-naphthalenyl)ethyl](methyl)amino]-2-oxoethyl}-4-[3-fluoro-4-(methyloxy)phenyl]-1-piperidinecarboxylate

### (Enantiomer 2)

DIPEA (290 μL) and O-(benzotriazol-1-yl)-N,N,N′N′-tetramethyluronium tetrafluoroborate (239 mg) were added to a solution of intermediate 40 (249 mg) in anhydrous DMF (4 mL) under a Nitrogen atmosphere. After stirring for 30 minutes, intermediate 15 (140 mg) was added. The mixture was stirred at rt overnight, then it was diluted with AcOEt, washed with aqueous std NaHCO₃, water and brine; then it was dried and evaporated under vacuum to give a crude product [T.I.c.: CH/AcOEt 1:1, Rf=0.50 (detection with ninhydrin)]. This intermediate was dissolved in dry DMF (5 mL) and, under a Nitrogen atmosphere and at 0° C., NaH 60% dispersion in mineral oil (53 mg) was added. The mixture was allowed to warm to rt and stirred under these conditions for 20 min. Then methyl iodide was added (0.41 mL) and the solution was stirred for 2 h at 50° C. Water and AcOEt were added; the organic phase separated and washed with brine, dried and evaporated under vacuum to give the title compound (372 mg) as white foam without any further purification.

T.I.c.: CH/AcOEt 1:1, Rf=0.62 (detection with ninhydrin).

MS (ES/+): m/z=591 [M+Na]⁺.

### EXAMPLE 1

### N-[(3-Chloro-1-naphthalenyl)methyl]-2-[4-(4-fluorophenyl)-4-piperidinyl]-N-methylacetamide

TFA (1.5 mL) was added to a solution of intermediate 66 (140 mg) in anhydrous DCM (6 mL) at 0° C. under a Nitrogen atmosphere. The reaction mixture was stirred 1 h before being concentrated in vacuo at 0° C. The residue was purified on a SCX-cartridge (loaded with DCM, washed with MeOH, eluted with NH3 0.25 M in MeOH, followed by MeOH). Solvent evaporation gave the title compound (90 mg) as a white foam.

T.I.c: DCM/MeOH 75:25, Rf=0.25 (detection with ninhydrine).

MS (ES/+): m/z=426 [M+H]⁺.

### EXAMPLE 2

### N-[(3-Chloro-1-naphthalenyl)methyl]-2-[4-(4-fluorophenyl)-1-methyl-4-piperidinyl]-N-methylacetamide

A solution of formaldehyde in water (37% w/w; 50 μl) was added to a stirred solution of Example 1 (80 mg) in CH₃CN (6 mL) under a Nitrogen atmosphere at rt. After 30 minutes sodium triacetoxyborohydride (50 mg) was added. The mixture was stirred for further 2 h then it was quenched with aqueous std NaHCO₃(5 mL) and extracted with AcOEt (3×50 mL). The combined organic phases were dried, concentrated in vacuo, and the residue purified on a SCX-cartridge (loaded with DCM, washed with MeOH, eluted with NH3 0.25 M in MeOH, followed by MeOH). Solvent evaporation gave the title compound (70 mg) as a white foam.

T.I.c.: DCM/MeOH 8:2, Rf=0.4 (detection with nyhydrine).

NMR (d₆-DMSO): δ 11 (ppm) 7.96-7.90 (m, 2H); 7.95 (s, 1H); 7.60-7.47 (m, 2H); 7.32 (dd, 2H); 7.08 (s, 1H); 6.92 (t, 2H); 4.75 (s, 2H); 2.70-2.01 (m, 16H).

MS (ES/+): m/z=440 [M+H]⁺.

Following the same procedure described to obtain example 1, example 3 was prepared.

### EXAMPLE 3

### N-[(3-Cyano-1-naphthalenyl)methyl]-2-[4-(4-fluorophenyl)-4-piperidinyl]-N-methylacetamide

Starting from intermediate 67 (81 mg), 50 mg of the title compound were obtained as a white solid.

NMR (CDCl₃): δ11 (ppm) 8.18 (m, 1H); 8.1-7.9 (m, 2H); 7.7-7.5 (m, 2H); 7.3-6.8 (m, 5H); 4.86 (s, 2H); 3.99 (t, 2H); 3.5-2.0 (m, 11H).

MS (ES/+): m/z=416 [M+H]⁺.

Following the same procedure described to obtain example 2, examples 4 was prepared.

### EXAMPLE 4

### N-[(3-cyano-1-naphthalenyl)methyl]-2-[4-(4-fluorophenyl)-1-methyl-4-piperidinyl]-N-methylacetamide

Starting from example 3 (45 mg), 40 mg of the title compound were obtained as a white solid.

T.I.c.: DCM/MeOH 9:1, Rf=0.11 (detection with ninhydrine).

MS (ES/+): m/z=430 [M+H]⁺.

NMR (d₆-DMSO): δ 11 (ppm) 8.50 (s, 1H); 8.15-8.00 (m, 2H); 7.75-7.70 (m, 2H); 7.40-7.25 (s+dd, 3H); 7.00-6.85 (t, 2H); 4.80 (s, 2H); 2.70 (s, 2H); 2.50-2.40 (m+s, 7H); 2.20-2.00 (m+s, 7H).

### EXAMPLE 5

### N-[(3-Cyano-1-naphthalenyl)methyl]-2-[4-(4-fluorophenyl)-1-methyl-4-piperidinyl]-N-methylacetamide Hydrochloride

Example 4 (37 mg) was dissolved in Et2O (2.0 mL), cooled to 0° C. and treated with HCl 1M solution in Et2O (1.0 mL). The mixture was stirred at 0° C. for 10 minutes, then it was concentrated in vacuo and the residue was triturated with pentane to give the title compound (35.0 mg) as a white solid.

NMR (d₆-DMSO): δ 12 (ppm) 9.8-9.6 (br, 1H); 8.50 (s, 1H); 8.1-7.6 (m, 4H); 7.50-7.25 (m, 3H); 7.00 (m, 2H); 4.76 (s, 2H); 3.4-2.4 (m, 14H); 2.1-1.8 (m, 2H).

Following the same procedure described to obtain example 1, example 6 was prepared.

### EXAMPLE 6

### N-[(3-Bromo-1-naphthalenyl)methyl]-2-[4-(4-fluorophenyl)-4-piperidinyl]-N-methylacetamide

Starting from intermediate 68 (58 mg), 30 mg of the title compound were obtained as a white solid.

T.I.c.: DCM/MeOH 7:3, Rf=0.12.

NMR (d₆-DMSO): δ 11 (ppm) 8.08 (d, 1H); 7.93 (d, 1H); 7.88 (d, 1H); 7.53-7.47 (m, 2H); 7.28 (dd, 2H); 7.23 (d, 1H); 6.89 (t, 2H); 4.73 (s, 2H); 2.68 (s, 2H); 2.8-1.8 (m, 8H); 2.42 (s, 3H).

MS (ES/+): m/z=469, 471 [M+H]⁺.

Following the same procedure described to obtain example 2, example 7 was prepared.

### EXAMPLE 7

### N-[(3-Bromo-1-naphthalenyl)methyl]-2-[4-(4-fluorophenyl)-1-methyl-4-piperidinyl]-N-methylacetamide

Starting from example 6 (27 mg), 19 mg of the title compound were obtained as a white solid.

T.I.c. DCM/MeOH 7:3, Rf=0.25.

NMR (d₆-DMSO): δ 11 (ppm) 8.08 (s, 1H); 7.92-7.88 (m, 2H); 7.53-7.46 (m, 2H); 7.29-6.85 (m, 5H); 4.72 (s, 2H); 2.7-1.9 (m, 16H).

MS (ES/+): m/z=483, 485 [M+H]⁺.

Following the same procedure described to obtain example 1, examples 8 was prepared.

### EXAMPLE 8

### N-[(5-bromo-1-benzofuran-7-yl)methyl]-2-[4-(4-fluorophenyl)-4-piperidinyl]-N-methylacetamide

Starting from intermediate 69 (164 mg), 62 mg of the title compound were obtained as a white solid.

NMR (d₆-DMSO): δ 11 (ppm) 8.02 (d, 1H); 7.76 (d, 1H); 7.38-7.31 (dd, 2H); 7.00-6.98 (m, 2H); 6.96 (d, 1H); 6.94 (d, 1H); 6-5 (vbs, 1H); 4.56 (s, 2H); 2.93 (m, 2H); 2.72 (s, 2H); 2.66 (m, 2H); 2.53 (s, 3H); 2.2-1.9 (m, 4H).

Following the same procedure described to obtain example 2, examples 9 was prepared.

### EXAMPLE 9

### N-[(5-bromo-1-benzofuran-7-yl)methyl]-2-[4-(4-fluorophenyl)-1-methyl-4-piperidinyl]-N-methylacetamide

Starting from example 8 (57 mg), 45 mg of the title compound were obtained as a white solid.

NMR (d₆-DMSO): δ 11 (ppm) 8.00 (d, 1H); 7.74 (d, 1H); 7.35-7.25 (m, 2H); 7.1-6.9 (m, 4H); 6.96 (d, 1H); 4.54 (s, 2H); 2.60 (2d, 2H); 2.5 (s, 3H); 2.6-2.0 (m, 8H); 2.07 (s, 3H).

MS (ES/+): m/z=473, 475 [M+H]⁺.

Following the same procedure described to obtain example 1, example 10 was prepared.

### EXAMPLE 10

### N-[1-(5-bromo-1-benzofuran-7-yl)ethyl]-2-[4-(4-fluorophenyl)-4-piperidinyl]-N-methylacetamide

Starting from intermediate 70 (93 mg), 71 mg of the title compound were obtained as a white solid.

NMR (CDCl₃): δ (ppm) 7.6 (s, 1H); 7.5 (s, 1H); 7.2 (m, 2H); 7.1 (s, 1H); 6.8 (t, 2H); 6.7 (s, 1H); 6.2 (q, 1H); 2.9 (m, 2H); 2.7 (m, 2H); 2.6 (s, 2H); 2.5-2.0 (m, 4H); 2.1 (s, 3H); 1.3 (d, 3H).

MS (ES/+): m/z=473, 475 [M+H]⁺.

Following the same procedure described to obtain example 2, example 11 was prepared.

### EXAMPLE 11

### N-[1-(5-bromo-1-benzofuran-7-yl)ethyl]-2-[4-(4-fluorophenyl)-1-methyl-4-piperidinyl]-N-methylacetamide

Starting from example 10 (50 mg), 32 mg of the title compound were obtained as a white solid.

MS (ES/+): m/z=487-489 [M+H]⁺.

NMR (CDCl₃): δ (ppm) 7.6 (s, 1H); 7.5 (s, 1H); 7.3 (m, 2H); 7.1 (s, 1H); 6.8 (t, 2H); 6.7 (s, 1H); 6.2 (q, 1H); 2.7-2.0 (m, 8H); 2.7 (s, 3H); 2.2 (s, 3H); 2.1 (s, 2H); 1.3 (d, 3H).

Following the same procedure described to obtain example 1, example 12 was prepared.

### EXAMPLE 12

### N-[(5-cyano-1-benzofuran-7-yl)methyl]-2-[4-(4-fluorophenyl)-4-piperidinyl]-N-methylacetamide

Starting from intermediate 71 (93 mg), 71 mg of the title compound were obtained as a white solid.

NMR (CDCl₃): δ (ppm) 7.8 (s, 1H); 7.6 (s, 1H); 7.4 (s, 1H); 7.3-7.1 (m, 3H); 6.8 (m, 2H); 4.6 (s, 2H); 3.0 (m, 2H); 2.7 (m, 2H); 2.6 (s, 2H); 2.5 (s, 3H); 2.3 (m, 2H); 2.1 (m, 2H).

MS (ES/+): m/z=406 [M+H]⁺.

Following the same procedure described to obtain example 2, example 13 was prepared.

### EXAMPLE 13

### N-[(5-cyano-1-benzofuran-7-yl)methyl]-2-[4-(4-fluorophenyl)-1-methyl-4-piperidinyl]-N-methylacetamide

Starting from Example 12 (66 mg), 45 mg of the title compound were obtained as a white solid.

MS (ES/+): m/z=420 [M+H]⁺.

NMR (CDCl₃): δ (ppm) 7.8 (s, 1H); 7.6 (s, 1H); 7.3 (s, 1H); 7.3-7.1 (m, 3H); 6.8 (m, 2H); 4.6 (s, 2H); 3.0-2.0 (m, 8H); 2.7 (s, 3H); 2.4 (s, 2H); 2.2 (s, 3H).

Following the same procedure described to obtain example 1, example 14 was prepared.

### EXAMPLE 14

### N-[1-(3-cyano-1-naphthalenyl)ethyl]-2-[4-(4-fluorophenyl)-4-piperidinyl]-N-methylacetamide

### (Enantiomer 1)

Starting from intermediate 75 (55 mg), 24 mg of the title compound were obtained as a white solid.

MS (ES/+): m/z=430 [M+H]⁺.

Following the same procedure described to obtain example 2, examples 15 was prepared.

### EXAMPLE 15

### N-[1-(3-cyano-1-naphthalenyl)ethyl]-2-[4-(4-fluorophenyl)-1-methyl-4-piperidinyl]-N-methylacetamide (Enantiomer 1)

Starting from example 14 (24 mg), 11 mg of the title compound were obtained as a white solid.

MS (ES/+): m/z=444 [M+H]⁺.

Following the same procedure described to obtain example 5, example 16 was prepared.

### EXAMPLE 16

### N-[1-(3-cyano-1-naphthalenyl)ethyl]-2-[4-(4-fluorophenyl)-1-methyl-4-piperidinyl]-N-methylacetamide Hydrochloride (Enantiomer 1)

Starting from example 15 (155 mg), 136 mg of the title compound were obtained as a white solid.

NMR (d₆-DMSO): δ (ppm) 9.69 (bs, 1H); 8.56 (s, 1H); 8.10 (d, 1H); 7.82 (bs, 1H); 7.76 (s, 1H); 7.7 (t, 1H); 7.61 (m, 1H); 7.4-6.9 (m, 4H); 6.31 (q, 1H); 2.9-2.0 (m, 16H); 1.33 (d, 3H).

Following the same procedure described to obtain example 1, example 17 was prepared.

### EXAMPLE 17

### N-[1-(3-cyano-1-naphthalenyl)ethyl]-2-[4-(4-fluoro-3-methylphenyl)-4-piperidinyl]-N-methylacetamide (Enantiomer 1)

Starting from intermediate 76 (42 mg), 14 mg of the title compound were obtained as a white solid.

MS (ES/+): m/z=444 [M+H]⁺.

Following the same procedure described to obtain example 2, example 18 was prepared.

### EXAMPLE 18

### N-[1-(3-cyano-1-naphthalenyl)ethyl]-2-[4-(4-fluoro-3-methylphenyl)-1-methyl-4-piperidinyl]-N-methylacetamide (Enantiomer 1)

Starting from example 17 (14 mg), 11 mg of the title compound were obtained as a white solid.

MS (ES/+): m/z=458 [M+H]⁺.

Following the same procedure described to obtain example 5, example 19 was prepared.

### EXAMPLE 19

### N-[1-(3-cyano-1-naphthalenyl)ethyl]-2-[4-(4-fluoro-3-methylphenyl)-1-methyl-4-piperidinyl]-N-methylacetamide Hydrochloride

### (Enantiomer 1)

Starting from example 18 (11 mg), 10 mg of the title compound were obtained as a white solid.

NMR (d₆-DMSO): δ (ppm) 9.69 (bs, 1H); 8.56 (s, 1H); 8.10 (d, 1H); 7.82 (bs, 1H); 7.76 (s, 1H); 7.7 (t, 1H); 7.61 (m, 1H); 7.4-6.9 (m, 3H); 6.31 (q, 1H); 2.9-2.0 (m, 19H); 1.33 (d, 3H).

MS (ES/+): m/z=458 [M-HCl+H]⁺.

Following the same procedure described to obtain example 1, example 20 was prepared.

### EXAMPLE 20

### N-[1-(3-cyano-1-naphthalenyl)ethyl]-2-[4-(4-fluorophenyl)-4-piperidinyl]-N-methylacetamide (Enantiomer 2)

Starting from intermediate 73 (61 mg), 47 mg of the title compound were obtained as a white solid.

MS (ES/+): m/z=430 [M+H]⁺.

Following the same procedure described to obtain example 2, example 21 was prepared.

### EXAMPLE 21

### N-[1-(3-cyano-1-naphthalenyl)ethyl]-2-[4-(4-fluorophenyl)-1-methyl-4-piperidinyl]-N-methylacetamide (Enantiomer 2)

Starting from example 20 (47 mg), 41 mg of the title compound were obtained as a white solid.

MS (ES/+): m/z=444 [M+H]⁺.

NMR (d₆-DMSO): δ (ppm) 8.55 (s, 1H); 8.08 (d, 1H); 7.87 (d, 1H); 7.74 (s, 1H); 7.7 (t, 1H); 7.64 (t, 1H); 7.35 (dd, 2H); 6.95 (t, 2H); 6.31 (q, 1H); 2.63 (d, 1H); 2.56 (d, 1H); 2.46 (bm, 2H); 2.25-2.0 (bm, 6H); 2.10 (s, 3H); 2.06 (s, 3H); 1.32 (d, 3H).

Following the same procedure described to obtain example 1, example 22 was prepared.

### EXAMPLE 22

### N-[1-(3-cyano-1-naphthalenyl)ethyl]-2-[4-(4-fluoro-3-methylphenyl)-4-piperidinyl]-N-methylacetamide (Enantiomer 2)

Starting from intermediate 74 (65 mg), 52 mg of the title compound were obtained as a white solid.

MS (ES/+): m/z=444 [M+H]⁺.

Following the same procedure described to obtain example 2, example 23 was prepared.

### EXAMPLE 23

### N-[1-(3-cyano-1-naphthalenyl)ethyl]-2-[4-(4-fluoro-3-methylphenyl)-1-methyl-4-piperidinyl]-N-methylacetamide (Enantiomer 2)

Starting from example 22 (52 mg), 38 mg of the title compound were obtained as a white solid.

MS (ES/+): m/z=458 [M+H]⁺.

NMR (d₆-DMSO): δ (ppm) 8.55 (s, 1H); 8.08 (d, 1H); 7.88 (d, 1H); 7.75 (s, 1H); 7.69 (t, 1H); 7.64 (t, 1H); 7.20 (dd, 1H); 7.15 (m, 1H); 6.89 (t, 1H); 6.31 (q, 1H); 2.62 (d, 1H); 2.52 (d, 1H); 2.45 (bm, 2H); 2.5-2.0 (bm, 6H); 2.10 (s, 3H); 2.09 (s, 3H); 2.01 (s, 3H); 1.31 (d, 3H).

Following the same procedure described to obtain example 1, example 24 was prepared.

### EXAMPLE 24

### N-[1-(5-cyano-1-benzofuran-7-yl)ethyl]-2-[4-(4-fluorophenyl)-4-piperidinyl]-N-methylacetamide

Starting from intermediate 72 (200 mg), 22 mg of the title compound were obtained as a yellowish oil.

MS (ES/+): m/z=420 [M+H]⁺.

NMR ((CDCl₃): δ (ppm) 7.86 (s, 1H); 7.69 (s, 1H); 7.33 (s, 1H); 7.25 (m, 2H); 6.89 (t, 2H); 6.82 (s, 1H); 6.23 (q, 1H); 2.59 (s, 2H); 2.17 (s, 3H); 3.2-2.0 (bm., 8H); 1.39 (d, 3H).

Following the same procedure described to obtain example 2, example 25 was prepared.

### EXAMPLE 25

### N-[1-(5-cyano-1-benzofuran-7-yl)ethyl]-2-[4-(4-fluorophenyl)-1-methyl-4-piperidinyl]-N-methylacetamide

Starting from example 24 (137 mg), 118 mg of the title compound were obtained as a white solid.

NMR (CDCl₃): δ (ppm) 7.86 (s, 1H); 7.68 (d, 1H); 7.33 (s, 1H); 7.26 (dd, 2H); 6.88 (t, 2H); 6.82 (d, 1H); 6.23 (q, 1H); 2.59 (s, 2H); 2.22 (s, 3H); 2.15 (s, 3H); 2.65-2.0 (bm, 8H); 1.38 (d, 3H).

Following the same procedure described to obtain example 1, example 26 was prepared.

### EXAMPLE 26

### N-[(3-cyano-6-fluoro-1-naphthalenyl)methyl]-2-[4-(4-fluorophenyl)-4-piperidinyl]-N-methylacetamide

Starting from intermediate 77 (100 mg), 75 mg of the title compound were obtained as a yellow oil without any chromatographic purification.

MS (ES/+): m/z=434 [M+H]⁺.

NMR (CDCl₃): δ (ppm) 8.11 (dd, 1H); 8.09 (s, 1H); 7.51 (dd, 1H); 7.39 (ddd, 1H); 7.27 (dd, 2H); 7.21 (s, 1H); 6.9 (t, 2H); 4.81 (s, 2H); 2.67 (s, 2H); 2.64-2.52 (bm, 2H); 3.43-2.07 (bm, 6H); 2.34 (s, 3H).

Following the same procedure described to obtain example 2, example 27 was prepared.

### EXAMPLE 27

### N-[(3-cyano-6-fluoro-1-naphthalenyl)methyl]-2-[4-(4-fluorophenyl)-1-methyl-4-Piperidinyl]-N-methylacetamide

Starting from example 26 (71 mg), 52 mg of the title compound were obtained as a yellow oil.

MS (ES/+): m/z=448 [M+H]⁺.

NMR (CDCl₃): δ (ppm) 8.11 (dd, 1H); 8.09 (s, 1H); 7.51 (dd, 1H); 7.39 (ddd, 1H); 7.27 (dd, 2H); 7.21 (s, 1H); 6.9 (t, 2H); 4.81 (s, 2H); 2.67 (s, 2H); 2.64-2.52 (bm, 2H); 3.43-2.07 (bm, 6H); 2.34 (s, 3H); 2.23 (s, 3H).

Following the same procedure described to obtain example 1, example 28 was prepared.

### EXAMPLE 28

### N-[1-(3-chloro-1-naphthalenyl)ethyl]-2-[4-(4-fluorophenyl)-4-piperidinyl]acetamide (Enantiomer 1)

Starting from intermediate 78 (61 mg), 39 mg of the title compound were obtained as a white foam.

MS (ES/+): m/z=425 [M+H]⁺.

NMR (CDCl₃): δ (ppm) 7.96 (m, 1H); 7.78 (m 1H); 7.77 (d, 1H); 7.53 (m, 2H); 7.17 (dd, 2H); 7.13 (d, 1H); 6.89 (t, 2H); 5.68 (m, 1H); 4.91 (d, 1H); 3.05 (m, 2H); 2.82 (m, 2H); 2.43 (2d, 2H); 2.5-2.0 (bm, 4H); 1.33 (d, 3H).

Following the same procedure described to obtain example 2, example 29 was prepared.

### EXAMPLE 29

### N-[1-(3-chloro-1-naphthalenyl)ethyl]-2-[4-(4-fluorophenyl)-1-methyl-4-piperidinyl]acetamide (Enantiomer 1)

Starting from example 28 (27 mg), 23 mg of the title compound were obtained as a white foam after chromatographic purification eluting with DCM 100% to DCM:MeOH=8:2.

MS (ES/+): m/z=439 [M+H]⁺.

NMR (CDCl₃): δ (ppm) 7.94 (m, 1H); 7.72 (d, 1H); 7.75 (m, 1H); 7.48 (m, 2H); 7.14 (dd, 2H); 7.08 (d, 1H); 6.83 (t, 2H); 5.64 (m, 1H); 4.77 (d, 1H); 2.7-2.5 (bm, 2H); 2.4 (2d, 2H); 2.5-2.0 (bm, 6H); 2.23 (s, 3H); 1.27 (d, 3H).

Following the same procedure described to obtain example 1, example 30 was prepared.

### EXAMPLE 30

### N-[1-(3-chloro-1-naphthalenyl)ethyl]-2-[4-(4-fluorophenyl)-4-piperidinyl]acetamide (Enantiomer 2)

Starting from intermediate 79 (60 mg), 34 mg of the title compound were obtained as a white foam.

MS (ES/+): m/z=425 [M+H]⁺.

NMR (CDCl₃): δ (ppm) 7.96 (m, 1H); 7.78 (m 1H); 7.77 (d, 1H); 7.53 (m, 2H); 7.17 (dd, 2H); 7.13 (d, 1H); 6.89 (t, 2H); 5.68 (m, 1H); 4.91 (d, 1H); 3.05 (m, 2H); 2.82 (m, 2H); 2.43 (2d, 2H); 2.5-2.0 (bm, 4H); 1.33 (d, 3H).

Following the same procedure described to obtain example 2, example 31 was prepared.

### EXAMPLE 31

### N-[1-(3-chloro-1-naphthalenyl)ethyl]-2-[4-(4-fluorophenyl)-1-methyl-4-piperidinyl]acetamide (Enantiomer 2)

Starting from example 30 (22 mg), 21 mg of the title compound were obtained as a white foam after chromatographic purification eluting with DCM 100% to DCM:MeOH=8:2.

MS (ES/+): m/z=439 [M+H]⁺.

NMR (CDCl₃): δ (ppm) 7.94 (m, 1H); 7.72 (d, 1H); 7.75 (m, 1H); 7.48 (m, 2H); 7.14 (dd, 2H); 7.08 (d, 1H); 6.83 (t, 2H); 5.64 (m, 1H); 4.77 (d, 1H); 2.7-2.5 (bm, 2H); 2.4 (2d, 2H); 2.5-2.0 (bm, 6H); 2.23 (s, 3H); 1.27 (d, 3H).

Following the same procedure described to obtain example 1, example 32 was prepared.

### EXAMPLE 32

### 2-[4-(1,3-benzodioxol-5-yl)-4-piperidinyl]-N-[1-(3,5-dichlorophenyl)ethyl]-N-methylacetamide (Enantiomer 1)

Starting from intermediate 82 (107 mg), 85 mg of the title compound were obtained as a white foam without any chromatographic purification.

MS (ES/+): m/z=449 [M+H]⁺.

Following the same procedure described to obtain example 2, example 33 was prepared.

### EXAMPLE 33

### 2-[4-(1,3-benzodioxol-5-yl)-1-methyl-4-piperidinyl]-N-[1-(3,5-dichlorophenyl)ethyl]-N-methylacetamide (Enantiomer 1)

Starting from example 32 (85 mg), 60 mg of the title compound were obtained as a white foam after chromatographic purification eluting with DCM 100% to DCM:MeOH=8:2.

MS (ES/+): m/z=463 [M+H]⁺.

NMR (CDCl₃): δ (ppm) 7.21 (s, 1H); 6.98 (s, 2H); 6.83-6.73 (m, 3H); 5.95-5.80 (s+q, 2/1H); 2.7-2.0 (m, 8H); 2.58 (s, 2H); 2.24 (s, 3H); 2.11 (s, 3H); 1.24 (d; 3H).

Following the same procedure described to obtain example 1, example 34 was prepared.

### EXAMPLE 34

### 2-[4-(1,3-benzodioxol-5-yl)-4-piperidinyl]-N-[1-(3,5-dibromophenyl)ethyl]-N-methylacetamide (Enantiomer 1)

Starting from intermediate 83 (128 mg), 106 mg of the title compound were obtained as a white foam without any chromatographic purification.

MS (ES/+): m/z=539 [M+H]⁺.

Following the same procedure described to obtain example 2, example 35 was prepared.

### EXAMPLE 35

### 2-[4-(1,3-benzodioxol-5-yl)-1-methyl-4-piperidinyl]-N-[1-(3,5-dibromophenyl)ethyl]-N-methylacetamide (Enantiomer 1)

Starting from example 34 (96 mg), 79 mg of the title compound were obtained as a white foam after chromatographic purification eluting with DCM 100% to DCM:MeOH=8:2.

MS (ES/+): m/z=553 [M+H]⁺.

NMR (CDCl₃): δ (ppm) 7.5 (s, 1H); 7.2 (s, 2H); 6.8-6.7 (m, 3H); 5.9-5.8 (s+q, 2/1H); 2.7-2.0 (m, 8H); 2.6 (s, 2H); 2.2 (s, 3H); 2.1 (s, 3H); 1.2 (d, 3H).

Following the same procedure described to obtain example 1, example 36 was prepared.

### EXAMPLE 36

### N-[1-(3-cyano-1-naphthalenyl)ethyl]-2-[4-[3-fluoro-4-(methyloxy)phenyl]-4-piperidinyl]-N-methylacetamide (Enantiomer 1)

Starting from intermediate 92 (276 mg), 193 mg of the title compound were obtained as a white foam.

MS (ES/+): m/z=460 [M+H]⁺.

NMR (CDCl₃): δ (ppm) 8.15 (s, 1H); 7.88 (m, 2H); 7.59 (m, 2H); 7.47 (s, 1H); 7.00 (m, 2H); 6.75 (t, 1H); 6.48 (q, 1H); 3.83 (s, 3H); 3.14 (m, 2H); 2.86 (m, 2H); 2.54 (s, 2H); 2.6-2.35 (bm, 2H); 2.25-2.05 (m, 2H); 1.97 (s, 3H); 1.38 (d, 3H).

Following the same procedure described to obtain example 2, example 37 was prepared.

### EXAMPLE 37

### N-[1-(3-cyano-1-naphthalenyl)ethyl]-2-{4-[3-fluoro-4-(methyloxy)phenyl]-1-methyl-4-piperidinyl}-N-methylacetamide (Enantiomer 1)

Starting from example 36 (164 mg), 88 mg of the title compound were obtained as a white foam after chromatographic purification eluting with DCM 100% to DCM:MeOH=7:3.

MS (ES/+): m/z=474 [M+H]⁺.

NMR (CDCl₃): δ (ppm) 8.15 (s, 1H); 7.94 (d, 1H); 7.87 (m, 1H); 7.59 (m, 2H); 7.46 (s, 1H); 7.01 (m, 2H); 6.71 (t, 1H); 6.51 (q, 1H); 3.82 (s, 3H); 2.7-2.5 (bm, 2H); 2.53 (s, 2H); 2.5-2.0 (bm, 6H); 2.22 (s, 3H); 1.94 (s, 3H); 1.38 (d, 3H).

Following the same procedure described to obtain example 1, example 38 was prepared.

### EXAMPLE 38

### N-[1-(3-cyano-1-naphthalenyl)ethyl]-2-{4-[3-fluoro-4-(methyloxy)phenyl]-4-piperidinyl}-N-methylacetamide (Enantiomer 2)

Starting from intermediate 93 (300 mg), 182 mg of the title compound were obtained as a white foam.

MS (ES/+): m/z=460 [M+H]⁺.

NMR (CDCl₃): δ (ppm) 8.15 (s, 1H); 7.88 (m, 2H); 7.59 (m, 2H); 7.47 (s, 1H); 7.00 (m, 2H); 6.75 (t, 1H); 6.48 (q, 1H); 3.83 (s, 3H); 3.14 (m, 2H); 2.86 (m, 2H); 2.54 (s, 2H); 2.6-2.35 (bm, 2H); 2.25-2.05 (m, 2H); 1.97 (s, 3H); 1.38 (d, 3H).

Following the same procedure described to obtain example 2, example 39 was prepared.

### EXAMPLE 39

### N-[1-(3-cyano-1-naphthalenyl)ethyl]-2-{4-[3-fluoro-4-(methyloxy)phenyl]-1-methyl-4-piperidinyl}-N-methylacetamide (Enantiomer 2)

Starting from example 38 (152 mg), 133 mg of the title compound were obtained as a white foam after chromatographic purification eluting with DCM 100% to DCM:MeOH=7:3.

MS (ES/+): m/z=474 [M+H]⁺.

NMR (CDCl₃): δ (ppm) 8.15 (s, 1H); 7.94 (d, 1H); 7.87 (m, 1H); 7.59 (m, 2H); 7.46 (s, 1H); 7.01 (m, 2H); 6.71 (t, 1H); 6.51 (q, 1H); 3.82 (s, 3H); 2.7-2.5 (bm, 2H); 2.53 (s, 2H); 2.5-2.0 (bm, 6H); 2.22 (s, 3H); 1.94 (s, 3H); 1.38 (d, 3H).

Following the same procedure described to obtain example 1, example 40 was prepared.

### EXAMPLE 40

### N-[(3-cyano-1-naphthalenyl)methyl]-2-{4-[3-fluoro-4-(methyloxy)phenyl]-4-piperidinyl}-N-methylacetamide

Starting from intermediate 93 (240 mg), 164 mg of the title compound were obtained as a white foam.

MS (ES/+): m/z=446 [M+H]⁺.

NMR (CDCl₃): δ (ppm) 8.15 (s, 1H); 8.1-8.0 (bm, 1H); 7.89 (m, 1H); 7.63 (m, 2H); 7.24 (s, 1H); 6.98 (m, 2H); 6.79 (t, 1H); 4.85 (s, 2H); 3.83 (s, 3H); 3.2 (bm, 2H); 2.9 (m, 2H); 2.66 (s, 2H); 2.6-2.1 (bm, 4H); 2.39 (s, 3H).

Following the same procedure described to obtain example 2, example 41 was prepared.

### EXAMPLE 41

### N-[(3-cyano-1-naphthalenyl)methyl]-2-{4-[3-fluoro-4-(methyloxy)phenyl]-1-methyl-4-piperidinyl}-N-methylacetamide

Starting from example 40 (132 mg), 111 mg of the title compound were obtained as a white foam after chromatographic purification eluting with DCM 100% to DCM:MeOH=7:3.

MS (ES/+): m/z=460 [M+H]⁺.

NMR (CDCl₃): δ (ppm) 8.15 (s, 1H); 8.07 (d, 1H); 7.90 (d, 1H); 7.64 (m, 2H); 7.62 (s, 1H); 7.1-6.95 (m, 2H); 6.74 (t, 1H); 4.85 (s, 2H); 3.81 (s, 3H); 2.64 (s, 2H); 2.6 (bm, 2H); 2.5-2.0 (bm, 6H); 2.34 (s, 3H); 2.22 (s, 3H).

Following the same procedure described to obtain example 1, example 42 was prepared.

### EXAMPLE 42

### N-[1-(3-chloro-1-naphthalenyl)ethyl]-2-{4-[3-fluoro-4-(methyloxy)phenyl]-4-piperidinyl}-N-methylacetamide (Enantiomer 1)

Starting from i intermediate 140 (396 mg), 230 mg of the title compound were obtained as a white foam.

MS (ES/+): m/z=469 [M+H]⁺.

Following the same procedure described to obtain example 2, example 43 was prepared.

### EXAMPLE 43

### N-[1-(3-chloro-1-naphthalenyl)ethyl]-2-{4-[3-fluoro-4-(methyloxy)phenyl]-1-methyl-4-piperidinyl}-N-methylacetamide (Enantiomer 1)

Starting from example 42 (195 mg), 180 mg of the title compound were obtained as a white foam after chromatographic purification eluting with DCM 100% to DCM:MeOH=7:3.

MS (ES/+): m/z=483 [M+H]⁺.

NMR (CDCl₃): δ (ppm) 7.9 (d, 1H); 7.73 (s, 1H); 7.75 (d, 1H); 7.36 (m, 2H); 7.3 (s, 1H); 7.03 (dd, 1H); 6.97 (d, 1H); 6.64 (t, 1H); 6.50 (q, 1H); 3.80 (s, 3H); 2.7-2.5 (bm, 2H); 2.53 (s, 2H); 2.5-2.0 (bm, 6H); 2.22 (s, 3H); 1.92 (s, 3H); 1.35 (d, 3H).

Following the same procedure described to obtain example 1, example 44 was prepared.

### EXAMPLE 44

### N-[1-(3-chloro-1-naphthalenyl)ethyl]-2-{4-[3-fluoro-4-(methyloxy)phenyl]-4-piperidinyl}-N-methylacetamide (Enantiomer 2)

Starting from intermediate 141 (372 mg), 226 mg of the title compound were obtained as a white foam.

MS (ES/+): m/z=469 [M+H]⁺.

NMR (CDCl₃): δ (ppm) 7.85 (d, 1H); 7.74 (s, 1H); 7.7 (d, 1H); 7.46 (m, 2H); 7.3 (s, 1H); 7.03 (dd, 1H); 6.97 (d, 1H); 6.66 (t, 1H); 6.48 (q, 1H); 3.80 (s, 3H); 3.06 (bm, 2H); 2.81 (q, 2H); 2.54 (s, 2H); 2.5-2.25 (bm, 2H); 2.25-1.95 (bm, 2H); 1.94 (s, 3H); 1.36 (d, 3H).

Following the same procedure described to obtain example 2, example 45 was prepared.

### EXAMPLE 45

### N-[1-(3-chloro-1-naphthalenyl)ethyl]-2-{4-[3-fluoro-4-(methyloxy)phenyl]-1-methyl-4-piperidinyl}-N-methylacetamide (Enantiomer 2)

Starting from example 44 (192 mg), 176 mg of the title compound were obtained as a white foam after chromatographic purification eluting with DCM 100% to DCM:MeOH=7:3.

MS (ES/+): m/z=483 [M+H]⁺.

NMR (CDCl₃): δ (ppm) 7.9 (d, 1H); 7.73 (s, 1H); 7.75 (d, 1H); 7.36 (m, 2H); 7.3 (s, 1H); 7.03 (dd, 1H); 6.97 (d, 1H); 6.64 (t, 1H); 6.50 (q, 1H); 3.80 (s, 3H); 2.7-2.5 (bm, 2H); 2.53 (s, 2H); 2.5-2.0 (bm, 6H); 2.22 (s, 3H); 1.92 (s, 3H); 1.35 (d, 3H).

Following the same procedure described to obtain example 1, example 46 was prepared.

### EXAMPLE 46

### N-[1-(3-chloro-1-naphthalenyl)ethyl]-2-[4-(4-cyanophenyl)-4-piperidinyl]-N-methylacetamide (Enantiomer 2)

Starting from intermediate 81 (31 mg), 13 mg of the title compound were obtained as a white foam.

NMR (CDCl₃): δ (ppm) 7.8 (d, 1H); 7.78 (d, 2H); 7.54 (d, 2H); 7.48-7.55 (m, 3H); 7.41 (tt, 1H); 7.35 (d, 1H); 6.47 (q, 1H); 3.0 (m, 2H); 2.79 (m, 2H); 2.67 (s, 2H); 2.36 (bm, 2H); 2.14 (m, 2H); 2.13 (s, 3H); 1.4 (d, 3H).

Following the same procedure described to obtain example 1, example 47 was prepared.

### EXAMPLE 47

### N-[1-(3-chloro-1-naphthalenyl)ethyl]-2-[4-(4-cyanophenyl)-4-piperidinyl]-N-methylacetamide (Enantiomer 1)

Starting from intermediate 118 (184 mg), 55 mg of the title compound were obtained as a white foam.

NMR (CDCl₃): δ (ppm) 7.8 (d, 1H); 7.78 (d, 2H); 7.54 (d, 2H); 7.48-7.55 (m, 3H); 7.41 (tt, 1H); 7.35 (d, 1H); 6.47 (q, 1H); 3.0 (m, 2H); 2.79 (m, 2H); 2.67 (s, 2H); 2.36 (bm, 2H); 2.14 (m, 2H); 2.13 (s, 3H); 1.4 (d, 3H).

Following the same procedure described to obtain example 2, example 48 was prepared.

### EXAMPLE 48

### N-[1-(3-chloro-1-naphthalenyl)ethyl]-2-[4-(4-cyanophenyl)-1-methyl-4-piperidinyl]-N-methylacetamide (Enantiomer 1)

Starting from example 47 (21 mg), 23 mg of the title compound were obtained as a white foam without any chromatographic purification.

MS (ES/+): m/z=460 [M+H]⁺.

NMR (CDCl₃): δ (ppm) 7.7 (d, 1H); 7.68 (d, 1H); 7.46-7.61 (bm, 3H); 7.38-7.47 (m, 3H); 7.26 (d, 1H); 7.24 (t, 1H); 6.32 (q, 1H); 2.93 (bm, 2H); 2.61 (d, 1H); 2.56 (d, 1H); 2.75-2.25 (bm, 6H); 2.4 (bs, 3H); 2.08 (s, 3H); 1.31 (d, 3H).

Following the same procedure described to obtain example 1, example 49 was prepared.

### EXAMPLE 49

### 2-[4-(1-benzofuran-5-yl)-4-piperidinyl]-N-[1-(3-chloro-1-naphthalenyl)ethyl]-N-methylacetamide (Enantiomer 1)

Starting from intermediate 125 (108 mg), 86 mg of the title compound were obtained as a white foam.

MS (ES/+): m/z=461 [M+H]⁺.

NMR (CDCl₃): δ (ppm) 7.84 (d, 1H); 7.75 (d, 1H); 7.75 (s, 1H); 7.64 (d, 1H); 7.55 (d, 1H); 7.51 (t, 1H); 7.37 (d, 1H); 7.34 (t, 1H); 7.25 (dd, 1H); 7.24 (d, 1H); 6.63 (dd, 1H); 6.48 (q, 1H); 3.19 (bm, 2H); 2.95 (m, 2H); 2.72 (bd, 1H); 2.69 (d, 1H); 2.62 (d, 1H); 2.57 (bd, 1H); 2.36 (bt, 1H); 2.24 (bt, 1H); 1.80 (s, 3H); 1.28 (d, 3H).

Following the same procedure described to obtain example 2, example 50 was prepared.

### EXAMPLE 50

### 2-[4-(1-benzofuran-5-yl)-1-methyl-4-piperidinyl]-N-[1-(3-chloro-1-naphthalenyl)ethyl]-N-methylacetamide (Enantiomer 1)

Starting from example 49 (60 mg), 44 mg of the title compound were obtained as a white foam after chromatographic purification eluting with DCM 100% to DCM:MeOH=9:1.

NMR (CDCl₃): δ (ppm) 7.8 (d, 1H); 7.75 (s, 1H); 7.74 (d, 1H); 7.64 (d, 1H); 7.58 (d, 1H); 7.5 (t, 1H); 7.39 (d, 1H); 7.33 (t, 1H); 7.3 (d, 1H); 7.24 (d, 1H); 6.64 (d, 1H); 6.46 (q, 1H); 2.97 (bm, 2H); 2.8-2.5 (bm, 2H); 2.68 (d, 1H); 2.63 (d, 1H); 2.7-2.3 (bm, 4H); 2.42 (bs, 3H); 1.82 (s, 3H); 1.27 (d, 3H).

Following the same procedure described to obtain example 1, example 51 was prepared.

### EXAMPLE 51

### 2-[4-(1-benzofuran-5-yl)-4-piperidinyl]-N-[1-(3-chloro-1-naphthalenyl)ethyl]-N-methylacetamide (Enantiomer 2)

Starting from intermediate 126 (51 mg), 35 mg of the title compound were obtained as a white foam.

NMR (CDCl₃): δ (ppm) 7.84 (d, 1H); 7.75 (d, 1H); 7.75 (s, 1H); 7.64 (d, 1H); 7.55 (d, 1H); 7.51 (t, 1H); 7.37 (d, 1H); 7.34 (t, 1H); 7.25 (dd, 1H); 7.24 (d, 1H); 6.63 (dd, 1H); 6.48 (q, 1H); 3.19 (bm, 2H); 2.95 (m, 2H); 2.72 (bd, 1H); 2.69 (d, 1H); 2.62 (d, 1H); 2.57 (bd, 1H); 2.36 (bt, 1H); 2.24 (bt, 1H); 1.80 (s, 3H); 1.28 (d, 3H).

Following the same procedure described to obtain example 2, example 52 was prepared.

### EXAMPLE 52

### 2-[4-(1-benzofuran-5-yl)-1-methyl-4-piperidinyl]-N-[1-(3-chloro-1-naphthalenyl)ethyl]-N-methylacetamide (Enantiomer 2)

Starting from example 51 (20 mg), 13 mg of the title compound were obtained as a white foam after chromatographic purification eluting with DCM 100% to DCM:MeOH=9:1.

NMR (CDCl₃): δ (ppm) 7.75 (s, 1H); 7.74 (d, 2H); 7.67 (d, 1H); 7.58 (bs, 1H); 7.49 (t, 1H); 7.42 (bd, 1H); 7.34-7.25 (m, 2H); 7.25 (d, 1H); 6.66 (bs, 1H); 6.43 (q, 1H); 3.19 (bm, 2H); 2.79 (bm, 2H); 2.71 (d, 1H); 2.65 (d, 1H); 2.8-2.45 (bm, 4H); 2.57 (bs, 3H); 1.88 (s, 3H); 1.29 (d, 3H).

Following the same procedure described to obtain example 1, example 53 was prepared.

### EXAMPLE 53

### N-[1-(3-chloro-1-naphthalenyl)ethyl]-2-[4-(2,3-dihydro-1-benzofuran-5-yl)-2-methyl-4-piperidinyl]-N-methylacetamide (Syn Isomer 1, Chain Enantiomer 1)

Starting from intermediate 137 (43 mg), 34 mg of the title compound were obtained as a white foam.

MS (ES/+): m/z=477 [M+H]⁺.

NMR (CDCl₃): δ (ppm) 7.92 (d, 1H); 7.79 (s, 1H); 7.77 (d, 1H); 7.55 (t, 1H); 7.51 (t, 1H); 7.33 (d, 1H); 7.08 (s, 1H); 7.01 (d, 1H); 6.59 (d, 1H); 6.52 (q, 1H); 4.52 (m, 2H); 3.43 (bm, 1H); 3.34 (bt, 1H); 3.01 (m, 1H); 2.92 (m, 1H); 2.78 (d, 1H); 2.75 (dm, 1H); 2.65 (d, 1H); 2.48 (bd, 1H); 1.96 (td, 1H); 1.91 (s, 3H); 1.76 (bt, 1H); 1.49 (d, 3H); 1.38 (d, 3H); 1.3 (t, 1H).

Following the same procedure described to obtain example 2, example 54 was prepared.

### EXAMPLE 54

### N-[1-(3-chloro-1-naphthalenyl)ethyl]-2-[4-(2,3-dihydro-1-benzofuran-5-yl)-1,2-dimethyl-4-piperidinyl]-N-methylacetamide (Syn Isomer 1, Chain Enantiomer 1)

Starting from example 53 (24 mg), 19 mg of the title compound were obtained as a white foam.

MS (ES/+): m/z=491 [M+H]⁺.

NMR (CDCl₃): δ (ppm) 7.85 (d, 1H); 7.69 (s, 1H); 7.68 (d, 1H); 7.45 (t, 1H); 7.42 (t, 1H); 7.23 (s, 1H); 6.97 (s, 1H); 6.92 (dd, 1H); 6.49 (d, 1H); 6.44 (q, 1H); 4.41 (m, 2H); 3.06 (bm, 1H); 2.93 (m, 1H); 2.89 (dm, 1H); 2.81 (m, 1H); 2.79 (m, 1H); 2.74 (d, 1H); 2.6 (dm, 1H); 2.52 (d, 1H); 2.46 (bm, 3H); 2.24 (bd, 1H); 1.99 (bm, 1H); 1.76 (s, 3H); 1.74 (bt, 1H); 1.29 (d, 3H); 1.26 (d, 3H).

Following the same procedure described to obtain example 1, example 55 was prepared.

### EXAMPLE 55

### N-[1-(3-chloro-1-naphthalenyl)ethyl]-2-[4-(2,3-dihydro-1-benzofuran-5-yl)-2-methyl-4-piperidinyl]-N-methylacetamide (Syn Isomer 2, Chain Enantiomer 1)

Starting from intermediate 138 (43 mg), 35 mg of the title compound were obtained as a white foam.

MS (ES/+): m/z=477 [M+H]⁺.

NMR (CDCl₃): δ (ppm) 7.89 (d, 1H); 7.78 (s, 1H); 7.77 (d, 1H); 7.54 (t, 1H); 7.51 (t, 1H); 7.32 (s, 1H); 7.13 (s, 1H); 7.05 (d, 1H); 6.63 (d, 1H); 6.52 (q, 1H); 4.54 (m, 2H); 3.48 (bm, 1H); 3.34 (bt, 1H); 3.21 (t, 1H); 3.04 (m, 1H); 2.98 (m, 1H); 2.83 (d, 1H); 2.79 (dm, 1H); 2.59 (d, 1H); 2.4 (bd, 1H); 2 (td, 1H); 1.88 (s, 3H); 1.54 (bt, 1H); 1.43 (d, 3H); 1.39 (d, 3H).

Following the same procedure described to obtain example 2, example 56 was prepared.

### EXAMPLE 56

### N-[1-(3-chloro-1-naphthalenyl)ethyl]-2-[4-(2,3-dihydro-1-benzofuran-5-yl)-1,2-dimethyl-4-piperidinyl]-N-methylacetamide (Syn Isomer 2, Chain Enantiomer 1)

Starting from example 55 (25 mg), 19 mg of the title compound were obtained as a white foam.

MS (ES/+): m/z=491 [M+H]⁺.

NMR (CDCl₃): δ (ppm) 7.86 (d, 1H); 7.73 (s, 1H); 7.72 (d, 1H); 7.49 (t, 1H); 7.46 (t, 1H); 7.28 (s, 1H); 7.03 (s, 1H); 6.98 (dd, 1H); 6.55 (d, 1H); 6.48 (q, 1H); 4.47 (m, 2H); 3.46 (m, 2H); 3.14 (bm, 1H); 2.96 (m, 1H); 2.88 (m, 1H); 2.77 (d, 1H); 2.65 (dm, 1H); 2.54 (d, 1H); 2.53 (bm, 3H); 2.31 (bm, 1H); 1.82 (bm, 1H); 1.78 (s, 3H); 1.37 (d, 3H); 1.35 (d, 3H).

Following the same procedure described to obtain example 1, examples 57 was prepared.

### EXAMPLE 57

### N-[1-(3-chloro-1-naphthalenyl)ethyl]-2-[2-ethenyl-4-(4-fluorophenyl)-4-piperidinyl]-N-methylacetamide (Syn Isomer 1. Chain Enantiomer 1)

Starting from intermediate 139 (8.5 mg), 3.4 mg of the title compound were obtained as a white foam.

MS (ES/+): m/z=465 [M+H]⁺.

NMR (CDCl₃): δ (ppm) 7.79 (d, 1H); 7.69 (s, 1H); 7.68 (d, 1H); 7.45 (td, 1H); 7.39 (td, 1H); 7.23 (d, 1H); 7.18 (dd, 2H); 6.76 (td, 2H); 6.41 (q, 1H); 5.88 (m, 1H); 5.32 (d, 1H); 5.16 (d, 1H); 3.63 (m, 1H); 3.25 (bd, 1H); 2.75 (d, 1H); 2.65 (bd, 1H); 2.61 (d, 1H); 2.4 (dm, 1H); 1.8-2.0 (m, 1H); 1.86 (s, 3H); 1.78 (tm, 1H); 1.63 (bt, 1H); 1.27 (d, 3H).

Following the same procedure described to obtain example 1, example 58 was prepared.

### EXAMPLE 58

### N-[1-(3-chloro-1-naphthalenyl)ethyl]-2-[4-(3-fluoro-4-methylphenyl)-4-piperidinyl]-N-methylacetamide (Enantiomer 1)

Starting from intermediate 119 (92 mg), 75 mg of the title compound were obtained as a white foam.

MS (ES/+): m/z=453 [M+H]⁺.

NMR (CDCl₃): δ (ppm) 7.81 (d, 1H); 7.72 (d, 1H); 7.71 (d, 1H); 7.47 (t, 1H); 7.4 (t, 1H); 7.28 (d, 1H); 7.04-6.90 (m, 3H); 6.45 (q, 1H); 3.02 (m, 2H); 2.79 (m, 2H); 2.54 (s, 2H); 2.45 (bd, 1H); 2.3 (bd, 1H); 2.21 (s, 3H); 2.17 (m, 1H); 2.01 (m, 1H); 1.94 (s, 3H); 1.33 (d, 3H).

Following the same procedure described to obtain example 2, example 59 was prepared.

### EXAMPLE 59

### N-[1-(3-chloro-1-naphthalenyl)ethyl]-2-[4-(3-fluoro-4-methylphenyl)-1-methyl-4-piperidinyl]-N-methylacetamide (Enantiomer 1)

Starting from example 58 (37 mg), 38 mg of the title compound were obtained as a white foam.

MS (ES/+): m/z=467 [M+H]⁺.

NMR (CDCl₃): δ (ppm) 7.83 (d, 1H); 7.82 (d, 1H); 7.77 (d, 1H); 7.53 (t, 1H); 7.41 (t, 1H); 7.33 (d, 1H); 7.1-6.99 (m, 3H); 6.49 (q, 1H); 2.98 (bm, 2H); 2.6 (s, 2H); 2.7-2.2 (bm, 6H); 2.41 (s, 3H); 2.28 (s, 3H); 1.99 (s, 3H); 1.39 (d, 3H).

Following the same procedure described to obtain example 1, example 60 was prepared.

### EXAMPLE 60

### N-[1-(3-chloro-1-naphthalenyl)ethyl]-2-[4-(3-fluoro-4-methylphenyl)-4-piperidinyl]-N-methylacetamide (Enantiomer 2)

Starting from intermediate 120 (90 mg), 72 mg of the title compound were obtained as a white foam.

MS (ES/+): m/z=453 [M+H]⁺.

NMR (CDCl₃): δ (ppm) 7.77 (d, 1H); 7.72 (d, 1H); 7.71 (d, 1H); 7.46 (t, 1H); 7.38 (t, 1H); 7.28 (d, 1H); 7.04-6.90 (m, 3H); 6.43 (q, 1H); 3.13 (m, 2H); 2.85 (m, 2H); 2.56 (d, 1H); 2.52 (d, 1H); 2.42 (bd, 1H); 2.26 (bt, 1H); 2.22 (s, 3H); 2.11 (bt, 1H); 2.01 (bm, 1H); 1.95 (s, 3H); 1.33 (d, 3H).

Following the same procedure described to obtain example 2, example 61 was prepared.

### EXAMPLE 61

### N-[1-(3-chloro-1-naphthalenyl)ethyl]-2-[4-(3-fluoro-4-methylphenyl)-1-methyl-4-piperidinyl]-N-methylacetamide (Enantiomer 2)

Starting from example 60 (30 mg), 24 mg of the title compound were obtained as a white foam.

MS (ES/+): m/z=467 [M+H]⁺.

NMR (CDCl₃): δ (ppm) 7.83 (d, 1H); 7.82 (d, 1H); 7.77 (d, 1H); 7.53 (t, 1H); 7.41 (t, 1H); 7.33 (d, 1H); 7.1-6.99 (m, 3H); 6.49 (q, 1H); 2.98 (bm, 2H); 2.6 (s, 2H); 2.7-2.2 (bm, 6H); 2.41 (s, 3H); 2.28 (s, 3H); 1.99 (s, 3H); 1.39 (d, 3H).

Following the same procedure described to obtain example 1, example 62 was prepared.

### EXAMPLE 62

### N-[1-(3-chloro-1-naphthalenyl)ethyl]-2-[4-(3-cyanophenyl)-4-piperidinyl]-N-methylacetamide (Enantiomer 1)

Starting from intermediate 121 (80 mg), 61 mg of the title compound were obtained as a white foam.

MS (ES/+): m/z=446 [M+H]⁺.

NMR (CDCl₃): δ (ppm) 7.79 (s, 1H); 7.78 (d, 1H); 7.78 (d, 1H); 7.69 (d, 1H); 7.66 (bd, 1H); 7.54 (d, 1H); 7.53 (t, 1H); 7.42 (td, 1H); 7.35 (t, 1H); 7.35 (d, 1H); 6.47 (q, 1H); 3.01 (bm, 2H); 2.81 (bm, 2H); 2.66 (s, 2H); 2.45-2.3 (bm, 2H); 2.2 (tm, 1H); 2.11 (s, 3H); 2.09 (m, 1H); 1.4 (d, 3H).

Following the same procedure described to obtain example 2, example 63 was prepared.

### EXAMPLE 63

### N-[1-(3-chloro-1-naphthalenyl)ethyl]-2-[4-(3-cyanophenyl)-1-methyl-4-piperidinyl]-N-methylacetamide (Enantiomer 1)

Starting from example 62 (40 mg), 41 mg of the title compound were obtained as a white foam.

MS (ES/+): m/z=460 [M+H]⁺.

NMR (CDCl₃): δ (ppm) 7.79 (s, 1H); 7.77 (d, 1H); 7.7 (bm, 1H); 7.71 (bs, 1H); 7.64 (bm, 1H); 7.7-7.6 (bm, 1H); 7.6-7.45 (bm, 2H); 7.52 (t, 1H); 7.36 (d, 1H); 6.37 (bm, 1H); 3.5-3.15 (bm, 2H); 2.9-2.5 (bm, 2H); 2.72 (d, 1H); 2.68 (bs, 3H); 2.7-2.5 (bm, 3H); 2.66 (d, 1H); 2.25 (bs, 3H); 2.04 (bm, 1H); 1.43 (d, 3H).

Following the same procedure described to obtain example 1, example 64 was prepared.

### EXAMPLE 64

### N-[1-(3-chloro-1-naphthalenyl)ethyl]-2-[4-(3-cyanophenyl)-4-piperidinyl]-N-methylacetamide (Enantiomer 2)

Starting from intermediate 122 (33 mg), 27 mg of the title compound were obtained as a white foam.

MS (ES/+): m/z=446 [M+H]⁺.

NMR (CDCl₃): δ (ppm) 7.79 (s, 1H); 7.78 (d, 1H); 7.78 (d, 1H); 7.69 (d, 1H); 7.66 (bd, 1H); 7.54 (d, 1H); 7.53 (t, 1H); 7.42 (td, 1H); 7.35 (t, 1H); 7.35 (d, 1H); 6.47 (q, 1H); 3.01 (bm, 2H); 2.81 (bm, 2H); 2.66 (s, 2H); 2.45-2.3 (bm, 2H); 2.2 (tm, 1H); 2.11 (s, 3H); 2.09 (m, 1H); 1.4 (d, 3H).

Following the same procedure described to obtain example 2, example 65 was prepared.

### EXAMPLE 65

### N-[1-(3-chloro-1-naphthalenyl)ethyl]-2-[4-(3-cyanophenyl)-1-methyl-4-piperidinyl]-N-methylacetamide (Enantiomer 2)

Starting from example 64 (13 mg), 14 mg of the title compound were obtained as a white foam.

MS (ES/+): m/z=460 [M+H]⁺.

NMR (CDCl₃): δ (ppm) 7.79 (s, 1H); 7.77 (d, 1H); 7.7 (bm, 1H); 7.71 (bs, 1H); 7.64 (bm, 1H); 7.7-7.6 (bm, 1H); 7.6-7.45 (bm, 2H); 7.52 (t, 1H); 7.36 (d, 1H); 6.37 (bm, 1H); 3.5-3.15 (bm, 2H); 2.9-2.5 (bm, 2H); 2.72 (d, 1H); 2.68 (bs, 3H); 2.7-2.5 (bm, 3H); 2.66 (d, 1H); 2.25 (bs, 3H); 2.04 (bm, 1H); 1.43 (d, 3H).

Following the same procedure described to obtain example 1, example 66 was prepared.

### EXAMPLE 66

### N-[1-(3-chloro-1-naphthalenyl)ethyl]-N-methyl-2-(4-phenyl-4-piperidinyl)acetamide (Enantiomer 1)

Starting from intermediate 123 (125 mg), 97 mg of the title compound were obtained as a white foam.

MS (ES/+): m/z=421 [M+H]⁺.

NMR (CDCl₃): δ (ppm) 7.87 (d, 1H); 7.77 (s, 1H); 7.76 (d, 1H); 7.52 (td, 1H); 7.46 (td, 1H); 7.16-7.36 (m, 6H); 6.49 (q, 1H); 3.21 (bt, 2H); 2.93 (bdd, 2H); 2.71 (bd, 1H); 2.64 (d, 1H); 2.59 (d, 1H); 2.54 (bd, 1H); 2.33 (bt, 1H); 2.2 (m, 1H); 1.88 (s, 3H); 1.35 (d, 3H).

Following the same procedure described to obtain example 2, example 67 was prepared.

### EXAMPLE 67

### N-[1-(3-chloro-1-naphthalenyl)ethyl]-N-methyl-2-(1-methyl-4-phenyl-4-piperidinyl)acetamide (Enantiomer 1)

Starting from example 66 (65 mg), 65 mg of the title compound were obtained as a white foam after chromatographic purification eluting with DCM 100% to DCM:MeOH=8:2.

MS (ES/+): m/z=435 [M+H]⁺.

NMR (CDCl₃): δ (ppm) 7.78 (d, 1H); 7.76 (d, 2H); 7.52 (t, 1H); 7.41 (t, 1H); 7.34 (s, 1H); 7.24-7.36 (m, 5H); 6.45 (q, 1H); 3.23 (bm, 2H); 2.78 (bm, 2H); 2.68 (d, 1H); 2.61 (s, 3H); 2.6 (d, 1H); 2.46-2.75 (bm, 4H); 1.96 (bs, 3H); 1.35 (d, 3H).

Following the same procedure described to obtain example 1, example 68 was prepared.

### EXAMPLE 68

### N-[1-(3-chloro-1-naphthalenyl)ethyl]-N-methyl-2-(4-phenyl-4-piperidinyl)acetamide (Enantiomer 2)

Starting from intermediate 124 (70 mg), 54 mg of the title compound were obtained as a white foam.

MS (ES/+): m/z=421 [M+H]⁺.

NMR (CDCl₃): δ (ppm) 7.81 (d, 1H); 7.77 (s, 1H); 7.76 (d, 1H); 7.52 (t, 1H); 7.43 (t, 1H); 7.16-7.36 (m, 6H); 6.46 (q, 1H); 3.48 (bt, 1H); 3.39 (bm, 1H); 3.09 (bt, 1H); 3.03 (bt, 1H); 2.89 (bd, 1H); 2.73 (bd, 1H); 2.62 (d, 1H); 2.57 (d, 1H); 2.44 (bt, 1H); 2.35 (bm, 1H); 1.91 (s, 3H); 1.34 (d, 3H).

Following the same procedure described to obtain example 2, example 69 was prepared.

### EXAMPLE 69

### N-[1-(3-chloro-1-naphthalenyl)ethyl]-N-methyl-2-(1-methyl-4-phenyl-4-piperidinyl)acetamide (Enantiomer 2)

Starting from example 68 (35 mg), 34 mg of the title compound were obtained as a white foam after chromatographic purification eluting with DCM 100% to DCM:MeOH=8:2.

MS (ES/+): m/z=435 [M+H]⁺.

NMR (CDCl₃): δ (ppm) 7.77 (s, 1H); 7.74 (d, 1H); 7.68 (d, 1H); 7.55 (t, 1H); 7.5 (t, 1H); 7.3-7.4 (m, 6H); 6.41 (q, 1H); 3.42 (bm, 2H); 3.04 (bd, 1H); 2.95 (bd, 1H); 2.5-2.8 (bm, 4H); 2.7 (d, 1H); 2.66 (s, 3H); 2.63 (d, 1H); 2.06 (s, 3H); 1.34 (d, 3H).

Following the same procedure described to obtain example 1, example 70 was prepared.

### EXAMPLE 70

### N-[1-(3-chloro-1-naphthalenyl)ethyl]-N-methyl-2-{4-[4-(methyloxy)phenyl]-4-piperidinyl}acetamide (Enantiomer 1)

Starting from intermediate 127 (47 mg), 40 mg of the title compound were obtained as a white foam.

NMR (CDCl₃): δ (ppm) 7.93 (d, 1H); 7.78 (d, 1H); 7.77 (d, 1H); 7.54 (t, 1H); 7.49 (t, 1H); 7.32 (d, 1H); 7.22 (d, 2H); 6.71 (d, 2H); 6.53 (q, 1H); 3.76 (s, 3H); 3.1 (bm, 2H); 2.87 (bm, 2H); 2.6 (d, 1H); 2.56 (d, 1H); 2.55 (bd, 1H); 2.41 (bd, 1H); 2.21 (bt, 1H); 2.11 (bt, 1H); 1.9 (s, 3H); 1.38 (d, 3H).

Following the same procedure described to obtain example 2, example 71 was prepared.

### EXAMPLE 71

### N-[1-(3-chloro-1-naphthalenyl)ethyl]-N-methyl-2-{1-methyl-4-[4-(methyloxy)phenyl]-4-piperidinyl}acetamide (Enantiomer 1)

Starting from example 70 (30 mg), 16 mg of the title compound were obtained as a white foam after chromatographic purification eluting with DCM:MeOH=98:2 to 8:2.

NMR (CDCl₃): δ (ppm) 7.85 (d, 1H); 7.78 (d, 1H); 7.76 (d, 1H); 7.53 (t, 1H); 7.44 (t, 1H); 7.32 (d, 1H); 7.23 (d, 2H); 6.75 (d, 2H); 6.5 (q, 1H); 3.78 (s, 3H); 3.05 (bm, 2H); 2.7 (bm, 2H); 2.63 (d, 1H); 2.6 (d, 1H); 2.51 (s, 3H); 2.35-2.15 (bm, 4H); 1.95 (s, 3H); 1.38 (d, 3H).

Following the same procedure described to obtain example 1, example 72 was prepared.

### EXAMPLE 72

### N-[1-(3-chloro-1-naphthalenyl)ethyl]-N-methyl-2-{4-[4-(methyloxy)phenyl]-4-piperidinyl}acetamide (Enantiomer 2)

Starting from intermediate 128 (57 mg), 46 mg of the title compound were obtained as a white foam.

NMR (CDCl₃): δ (ppm) 7.93 (d, 1H); 7.78 (d, 1H); 7.75 (d, 1H); 7.6-7.4 (dt, 2H); 7.31 (d, 1H); 7.22 (d, 2H); 6.72 (d, 2H); 6.53 (m, 1H); 3.76 (s, 3H); 3.15 (m, 2H); 2.9 (m, 2H); 2.59 (s, 2H); 2.5 (m, 2H); 2.0 (m, 2H); 1.9 (bs, 3H); 1.38 (s, 3H).

Following the same procedure described to obtain example 2, example 73 was prepared.

### EXAMPLE 73

### N-[1-(3-chloro-1-naphthalenyl)ethyl]-N-methyl-2-{1-methyl-4-[4-(methyloxy)phenyl]-4-piperidinyl}acetamide (Enantiomer 2)

Starting from example 72 (36 mg), 31 mg of the title compound were obtained as a white foam after chromatographic purification eluting with DCM:MeOH=98:2 to 8:2.

NMR (CDCl₃): δ (ppm) 7.89 (d, 1H); 7.77 (d, 1H); 7.76 (d, 1H); 7.54 (t, 1H); 7.49 (t, 1H); 7.32 (d, 1H); 7.23 (d, 2H); 6.73 (d, 2H); 6.51 (q, 1H); 3.77 (s, 3H); 2.89 (bm, 2H); 2.5-2.7 (bm, 2H); 2.61 (d, 1H); 2.56 (d, 1H); 2.4 (s, 3H); 2.5-2.2 (bm, 4H); 1.91 (s, 3H); 1.37 (d, 3H).

Following the same procedure described to obtain example 1, example 74 was prepared.

### EXAMPLE 74

### N-[1-(3-chloro-1-naphthalenyl)ethyl]-2-[4-(2,3-dihydro-1-benzofuran-5-yl)-4-piperidinyl]-N-methylacetamide (Enantiomer 1)

Starting from intermediate 129 (88 mg), 65 mg of the title compound were obtained as a white foam.

MS (ES/+): m/z—463 [M+H]⁺.

NMR (CDCl₃): δ (ppm) 7.83 (d, 1H); 7.78 (d, 1H); 7.76 (d, 1H); 7.53 (t, 1H); 7.51 (t, 1H); 7.33 (d, 1H); 7.33 (s, 1H); 7.11 (dd, 1H); 6.69 (d, 1H); 6.48 (q, 1H); 4.58 (m, 2H); 3.46 (bm, 1H); 3.37 (m, 1H); 3.15-2.9 (m, 4H); 2.9-2.6 (bm, 2H); 2.62 (d, 1H); 2.54 (d, 1H); 2.38 (td, 1H); 2.29 (bt, 1H); 1.96 (s, 3H); 1.38 (d, 3H).

Following the same procedure described to obtain example 2, example 75 was prepared.

### EXAMPLE 75

### N-[1-(3-chloro-1-naphthalenyl)ethyl]-2-[4-(2,3-dihydro-1-benzofuran-5-yl)-1-methyl-4-piperidinyl]-N-methylacetamide (Enantiomer 1)

Starting from example 74 (40 mg), 43 mg of the title compound were obtained without any further chromatographic purification.

MS (ES/+): m/z=477 [M+H]⁺.

NMR (CDCl₃): δ (ppm) 7.77 (d, 1H); 7.68 (d, 1H); 7.66 (d, 1H); 7.43 (t, 1H); 7.36 (t, 1H); 7.23 (d, 1H); 7.04 (s, 1H); 6.97 (dd, 1H); 6.58 (d, 1H); 6.41 (q, 1H); 4.46 (m, 2H); 2.85-2.30 (m, 4H); 2.6-2.3 (bm, 5H); 2.52 (d, 1H); 2.49 (d, 1H); 2.37 (s, 3H); 2.21 (bt, 1H); 1.83 (s, 3H); 1.28 (d, 3H).

Following the same procedure described to obtain example 1, example 76 was prepared.

### EXAMPLE 76

### N-[1-(3-chloro-1-naphthalenyl)ethyl]-2-[4-(2,3-dihydro-1-benzofuran-5-yl)-4-piperidinyl]-N-methylacetamide (Enantiomer 2)

Starting from intermediate 130 (118 mg), 88 mg of the title compound were obtained as a white foam.

MS (ES/+): m/z=463 [M+H]⁺.

NMR (CDCl₃): δ (ppm) 7.91 (d, 1H); 7.78 (d, 1H); 7.76 (d, 1H); 7.53 (t, 1H); 7.48 (t, 1H); 7.73 (d, 1H); 7.13 (s, 1H); 7.06 (dd, 1H); 6.67 (d, 1H); 6.53 (q, 1H); 4.56 (m, 2H); 3.46 (bm, 1H); 3.14 (m, 1H); 3.15-2.85 (m, 4H); 2.61 (d, 1H); 2.58 (bm, 1H); 2.56 (d, 1H); 2.41 (bd, 1H); 2.26 (bm, 1H); 2.14 (bm, 1H); 1.92 (s, 3H); 1.38 (d, 3H).

Following the same procedure described to obtain example 2, example 77 was prepared.

### EXAMPLE 77

### N-[1-(3-chloro-1-naphthalenyl)ethyl]-2-[4-(2,3-dihydro-1-benzofuran-5-yl)-1-methyl-4-piperidinyl]-N-methylacetamide (Enantiomer 2)

Starting from example 76 (40 mg), 29 mg of the title compound were obtained after chromatographic purification eluting with DCM:MeOH=98:2 to 8:2.

MS (ES/+): m/z=477 [M+H]⁺.

NMR (CDCl₃): δ (ppm) 7.86 (d, 1H); 7.78 (d, 1H); 7.76 (d, 1H); 7.53 (t, 1H); 7.46 (t, 1H); 7.33 (d, 1H); 7.14 (s, 1H); 7.07 (dd, 1H); 6.68 (d, 1H); 6.5 (q, 1H); 4.57 (m, 2H); 3.2-2.9 (m, 2H); 3.02 (m, 1H); 2.97 (m, 1H); 2.75-2.5 (m, 2H); 2.62 (d, 1H); 2.6-2.4 (m, 2H); 2.56 (d, 1H); 2.46 (bs, 3H); 2.5-2.25 (bm, 2H); 1.94 (s, 3H); 1.38 (d, 3H).

Following the same procedure described to obtain example 1, example 78 was prepared.

### EXAMPLE 78

### N-[1-(3-chloro-1-naphthalenyl)ethyl]-N-methyl-2-{2-methyl-4-[4-(methyloxy)phenyl]-4-piperidinyl}acetamide (Syn Isomer 1, Chain Enantiomer 1)

Starting from intermediate 135 (47 mg), 36 mg of the title compound were obtained as a white foam.

MS (ES/+): m/z=465 [M+H]⁺.

NMR (CDCl₃): δ (ppm) 7.94 (d, 1H); 7.78 (s, 1H); 7.77 (d, 1H); 7.55 (t, 1H); 7.51 (t, 1H); 7.31 (d, 1H); 7.19 (d, 2H); 6.65 (d, 2H); 6.52 (q, 1h); 3.74 (s, 3H); 3.3 (m, 2H); 2.79 (d, 1H); 2.69 (bd, 1H); 2.66 (d, 1H); 2.46 (dm, 1H); 1.88 (s, 3H); 1.83 (m, 1H); 1.57 (t, 1H); 1.37 (d, 3H); 1.35 (d, 3H); 1.25-1.4 (m, 1H).

Following the same procedure described to obtain example 2, example 79 was prepared.

### EXAMPLE 79

### N-[1-(3-chloro-1-naphthalenyl)ethyl]-2-{1,2-dimethyl-4-[4-(methyloxy)phenyl]-4-piperidinyl}-N-methylacetamide (Syn Isomer 1, Chain Enantiomer 1)

Starting from example 78 (25 mg), 23 mg of the title compound were obtained as a white foam.

MS (ES/+): m/z=479 [M+H]⁺.

NMR (CDCl₃): δ (ppm) 7.91 (d, 1H); 7.78 (s, 1H); 7.77 (d, 1H); 7.55 (t, 1H); 7.52 (t, 1H); 7.31 (d, 1H); 7.1 (d, 2H); 6.55 (d, 2H); 6.53 (q, 1 h); 3.74 (s, 3H); 3.25 (bm, 1H); 3. (bm, 1H); 2.7 (d, 1H); 2.69 (dm, 1H); 2.61 (d, 1H); 2.6 (bs, 3H); 2.45 (bd, 1H); 1.83 (t, 1H); 1.75 (s, 3H); 1.63 (m, 1H); 1.41 (d, 3H); 1.31 (m, 1H); 1.34 (d, 3H).

Following the same procedure described to obtain example 1, example 80 was prepared.

### EXAMPLE 80

### N-[1-(3-chloro-1-naphthalenyl)ethyl]-N-methyl-2-{2-methyl-4-[4-(methyloxy)phenyl]-4-piperidinyl}acetamide

Starting from intermediate 136 (50 mg), 23 mg of the title compound were obtained as a white foam.

MS (ES/+): m/z=465 [M+H]⁺.

NMR (CDCl₃): δ (ppm) 7.85 (d, 1H); 7.73 (s, 1H); 7.72 (d, 1H); 7.5 (t, 1H); 7.51 (t, 1H); 7.27 (d, 1H); 7.17 (d, 2H); 6.65 (d, 2H); 6.52 (q, 1h); 3.7 (s, 3H); 3.3-3.17 (m, 2H); 2.79 (d, 1H); 2.69 (bd, 1H); 2.6 (d, 1H); 2.46 (dm, 1H); 1.8 (s, 3H); 1.98 (m, 1H); 1.57 (t, 1H); 1.37 (d, 3H); 1.35 (d, 3H); 1.25-1.4 (m, 1H).

Following the same procedure described to obtain example 2, example 81 was prepared.

### EXAMPLE 81

### N-[1-(3-chloro-1-naphthalenyl)ethyl]-2-{1,2-dimethyl-4-[4-(methyloxy)phenyl]-4-piperidinyl}-N-methylacetamide (Syn Isomer 2, Chain Enantiomer 1)

Starting from example 80 (13 mg), 12 mg of the title compound were obtained as a white foam.

MS (ES/+): m/z=479 [M+H]⁺.

NMR (CDCl₃): δ (ppm) 7.91 (d, 1H); 7.78 (s, 1H); 7.77 (d, 1H); 7.55 (t, 1H); 7.52 (t, 1H); 7.31 (d, 1H); 7.18 (d, 2H); 6.65 (d, 2H); 6.53 (q, 1h); 3.74 (s, 3H); 3.18 (bm, 1H); 2.85 (bm, 1H); 2.8 (d, 1H); 2.69 (dm, 1H); 2.61 (d, 1H); 2.58 (bs, 3H); 2.42 (bd, 1H); 1.83 (t, 1H); 1.8 (s, 3H); 1.63 (m, 1H); 1.4 (d, 3H); 1.31 (m, 1H); 1.22 (d, 3H).

Following the same procedure described to obtain example 1, examples 82 and 83, 84 and 85 were prepared.

### EXAMPLE 82 AND 83

### N-[1-(3-cyano-1-naphthalenyl)ethyl]-N-methyl-2-(2-methyl-4-phenyl-4-piperidinyl)acetamide (Syn Isomer 1, Chain Enantiomer 1)

### N-[1-(3-cyano-1-naphthalenyl)ethyl]-N-methyl-2-(2-methyl-4-phenyl-4-piperidinyl)acetamide (Syn Isomer 2, Chain Enantiomer 1)

Starting from intermediate 99 (42 mg), 38 mg of a mixture of title compounds 82 and 83 was obtained as a white foam.

The mixture was then purified by semipreparative SFC (Gilson) chromatography [semipreparative conditions: Chiral column: CHIRALPAK AS-H, 25×2.1 cm; modifier: (Ethanol+0.1% Isopropylamine) 15% vs CO₂; flow rate=22 mL/min; pressure=196 bar; T=36° C.; UV wavelength: 220 nm; loop=2 mL] to obtain title compound 82 [analytical conditions: Chiral column: CHIRALPAK AS-H, 25×0.46 cm; modifier: (Ethanol+0.1% Isopropylamine) 15% vs CO₂; flow rate=2.5 mL/min; pressure=190 bar; T=35° C.; UV wavelength: 220 nm; loop=2 mL retention time=14.9 minutes] (14 mg) and title compound 83 (6 mg) [same analytical conditions retention time=18.7 minutes].

### EXAMPLE 82

MS (ES/+): m/z=426 [M+H]⁺.

NMR (CDCl₃): δ (ppm) 8.15 (s, 1H); 7.97 (d, 1H); 7.88 (dd, 1H); 7.65 (td, 1H); 7.6 (td, 1H); 7.43 (s, 1H); 7.3 (dd, 2H); 7.2-7.1 (m, 3H); 6.51 (q, 1H); 3.23 (m, 1H); 3.19 (dt, 1H); 3.14 (dm, 1H); 2.87 (d, 1H); 2.71 (bm, 1H); 2.63 (d, 1H); 2.35 (dm, 1H); 1.8 (s, 3H); 1.9-1.6 (m, 2H); 1.35 (d, 3H); 1.23 (d, 3H).

### EXAMPLE 83

MS (ES/+): m/z=426 [M+H]⁺.

NMR (CDCl₃): δ (ppm) 8.15 (s, 1H); 7.98 (dd, 1H); 7.88 (dd, 1H); 7.64 (td, 1H); 7.6 (td, 1H); 7.43 (s, 1H); 7.3 (dd, 2H); 7.2-7.1 (m, 3H); 6.5 (q, 1H); 3.23 (td, 1H); 3.19 (dt, 1H); 3.11 (m, 1H); 2.86 (d, 1H); 2.69 (dm, 1H); 2.65 (d, 1H); 2.37 (tdt, 1H); 1.85 (s, 3H); 1.9-1.7 (m, 1H); 1.66 (td, 1H); 1.32 (d, 3H); 1.17 (d, 3H).

### EXAMPLE 84 AND 85

### N-[1-(3-cyano-1-naphthalenyl)ethyl]-N-methyl-2-(2-methyl-4-phenyl-4-piperidinyl)acetamide (Syn Isomer 1, Chain Enantiomer 2)

### N-[1-(3-cyano-1-naphthalenyl)ethyl]-N-methyl-2-(2-methyl-4-phenyl-4-piperidinyl)acetamide (Syn Isomer 2, Chain Enantiomer 2)

Starting from intermediate 100 (36 mg), 28 mg of a mixture of title compounds 84 and 85 was obtained as a white foam (36 mg).

The mixture was then purified by semipreparative SFC (Gilson) chromatography [semipreparative conditions: Chiral column: CHIRALPAK AS-H, 25×2.1 cm; modifier: (Ethanol+0.1% Isopropylamine) 15% vs CO₂; flow rate=22 mL/min; pressure=196 bar; T=36° C.; UV wavelength: 220 nm; loop=2 mL] to obtain title compound 84 [analytical conditions: Chiral column: CHIRALPAK AS-H, 25×0.46 cm; modifier: (Ethanol+0.1% Isopropylamine) 15% vs CO₂; flow rate=2.5 mL/min; pressure=190 bar; T=35° C.; UV wavelength: 220 nm; loop=2 mL retention time=14.9 minutes] (13 mg) and title compound 85 (8 mg) [same analytical conditions retention time=18.7 minutes].

### EXAMPLE 84

MS (ES/+): m/z=426 [M+H]⁺.

NMR (CDCl₃): δ (ppm) 8.14 (s, 1H); 7.97 (d, 1H); 7.86 (dd, 1H); 7.63 (t, 1H); 7.59 (td, 1H); 7.42 (d, 1H); 7.29 (dd, 2H); 7.14 (m, 3H); 6.49 (q, 1H); 3.21 (td, 1H); 3.17 (m, 1H); 3.07 (m, 1H); 2.84 (d, 1H); 2.67 (bd, 1H); 2.63 (d, 1H); 2.35 (dm, 1H); 1.83 (s, 3H); 1.64 (td, 1H); 1.37 (td, 1H); 1.29 (d, 3H); 1.15 (d, 3H).

### EXAMPLE 85

MS (ES/+): m/z=426 [M+H]⁺.

NMR (CDCl₃): δ (ppm) 8.15 (s, 1H); 7.98 (dd, 1H); 7.88 (dd, 1H); 7.64 (td, 1H); 7.6 (td, 1H); 7.43 (s, 1H); 7.3 (dd, 2H); 7.2-7.1 (m, 3H); 6.5 (q, 1H); 3.23 (td, 1H); 3.19 (dt, 1H); 3.11 (m, 1H); 2.86 (d, 1H); 2.69 (dm, 1H); 2.65 (d, 1H); 2.37 (tdt, 1H); 1.85 (s, 3H); 1.9-1.7 (m, 1H); 1.66 (td, 1H); 1.32 (d, 3H); 1.17 (d, 3H).

Following the same procedure described to obtain example 2, examples 86, 87, 88, 89 were prepared.

### EXAMPLE 86

### N-[1-(3-cyano-1-naphthalenyl)ethyl]-2-(1,2-dimethyl-4-phenyl-4-piperidinyl)-N-methylacetamide (Syn Isomer 1, Chain Enantiomer 1)

Starting from example 82 (14 mg), 11 mg of the title compound were obtained as a white foam.

MS (ES/+): m/z=440 [M+H]⁺.

NMR (CDCl₃): δ (ppm) 8.16 (s, 1H); 7.96 (d, 1H); 7.88 (dd, 1H); 7.65 (td, 1H); 7.61 (td, 1H); 7.43 (s, 1H); 7.27 (dd, 2H); 7.1-7.2 (m, 3H); 6.51 (q, 1H); 3.15 (bm, 1H); 2.83 (d, 1H); 2.75-2.6 (m, 2H); 2.61 (d, 1H); 2.48 (bs, 3H); 2.4 (dm, 1H); 2.15 (bm, 1H); 1.77 (s, 3H); 1.73 (bm, 1H); 1.36 (d, 3H); 1.34 (d, 3H); 1.31 (bm, 1H).

### EXAMPLE 87

### N-[1-(3-cyano-1-naphthalenyl)ethyl]-2-(1,2-dimethyl-4-phenyl-4-piperidinyl)-N-methylacetamide (Syn Isomer 2, Chain Enantiomer 1)

Starting from example 83 (6 mg), 4 mg of the title compound were obtained as a white foam.

MS (ES/+): m/z=440 [M+H]⁺.

NMR (CDCl₃): δ (ppm) 8.15 (s, 1H); 7.98 (dd, 1H); 7.87 (dd, 1H); 7.64 (td, 1H); 7.6 (td, 1H); 7.43 (s, 1H); 7.28 (dd, 2H); 7.1-7.2 (m, 3H); 6.50 (q, 1H); 2.97 (dt, 1H); 2.84 (d, 1H); 2.69 (dm, 1H); 2.67 (m, 1H); 2.62 (d, 1H); 2.42 (m, 1H); 2.38 (s, 3H); 2.31 (dt, 1H); 1.94 (td, 1H); 1.83 (s, 3H); 1.66 (td, 1H); 1.32 (d, 3H); 1.2 (d, 3H).

### EXAMPLE 88

### N-[1-(3-cyano-1-naphthalenyl)ethyl]-2-(1,2-dimethyl-4-phenyl-4-piperidinyl)-N-methylacetamide (Syn Isomer 1, Chain Enantiomer 2)

Starting from Example 84 (13 mg), 9 mg of the title compound were obtained as a white foam.

MS (ES/+): m/z=440 [M+H]⁺.

NMR (CDCl₃): δ (ppm) 8.15 (s, 1H); 7.95 (dd, 1H); 7.87 (dd, 1H); 7.64 (td, 1H); 7.59 (td, 1H); 7.43 (s, 1H); 7.29 (dd, 2H); 7.13 (m, 3H); 6.50 (q, 1H); 2.93 (dt, 1H); 2.84 (d, 1H); 2.63 (dm, 1H); 2.59 (d, 1H); 2.57 (dm, 1H); 2.51 (tm, 1H); 2.38 (s, 3H); 2.36 (dm 1H); 2.01 (td, 1H); 1.79 (s, 3H); 1.54 (td, 1H); 1.35 (d, 3H); 1.23 (d, 3H).

### EXAMPLE 89

### N-[1-(3-cyano-1-naphthalenyl)ethyl]-2-(1,2-dimethyl-4-phenyl-4-piperidinyl)-N-methylacetamide (Syn Isomer 2, Chain Enantiomer 2)

Starting from example 85 (8 mg), 5 mg of the title compound were obtained as a white foam.

MS (ES/+): m/z=440 [M+H]⁺.

NMR (CDCl₃): δ (ppm) 8.15 (s, 1H); 7.98 (dd, 1H); 7.87 (dd, 1H); 7.64 (td, 1H); 7.6 (td, 1H); 7.43 (s, 1H); 7.28 (dd, 2H); 7.1-7.2 (m, 3H); 6.5 (q, 1H); 2.97 (dt, 1H); 2.84 (d, 1H); 2.69 (dm, 1H); 2.67 (m, 1H); 2.62 (d, 1H); 2.42 (m, 1H); 2.38 (s, 3H); 2.31 (dt, 1H); 1.94 (td, 1H); 1.83 (s, 3H); 1.66 (td, 1H); 1.32 (d, 3H); 1.2 (d, 3H).

Following the same procedure described to obtain example 1, examples 90, 91 were prepared.

### EXAMPLE 90

### N-[1-(3-cyano-1-naphthalenyl)ethyl]-2-[4-(4-fluorophenyl)-2-methyl-4-piperidinyl]-N-methylacetamide (Syn Isomer Chain Enantiomer 1)

Starting from intermediate 101 (36 mg), 25 mg of the title compounds was obtained as a white foam.

MS (ES/+): m/z=444 [M+H]⁺.

### EXAMPLE 91

### N-[1-(3-cyano-1-naphthalenyl)ethyl]-2-[4-(4-fluorophenyl)-2-methyl-4-piperidinyl]-N-methylacetamide (Syn Isomer, Chain Enantiomer 2)

Starting from intermediate 102 (74 mg), 55 mg of the title compounds was obtained as a white foam.

MS (ES/+): m/z=444 [M+H]⁺.

Following the same procedure described to obtain example 2, examples 92 and 93, 94 and 95 were prepared.

### EXAMPLE 92 AND 93

### N-[1-(3-cyano-1-naphthalenyl)ethyl]-2-[4-(4-fluorophenyl)-1,2-dimethyl-4-piperidinyl]-N-methylacetamide (Syn Isomer 1, Chain Enantiomer 1)

### N-[1-(3-cyano-1-naphthalenyl)ethyl]-2-[4-(4-fluorophenyl)-1,2-dimethyl-4-piperidinyl]-N-methylacetamide (Syn Isomer 2, Chain Enantiomer 1)

Starting from example 90 (25 mg), 14 mg of the mixture of title compounds was obtained as a white foam.

The mixture was then purified by semipreparative SFC (Gilson) chromatography [semipreparative conditions: Chiral column: CHIRALPAK AS-H, 25×2.1 cm; modifier: (Ethanol+0.1% Isopropylamine) 5% vs CO₂; flow rate=22 mL/min; pressure=192 bar; T=36° C.; UV wavelength: 220 nm; loop=1 mL; injection: 7.5 mg each injection] to obtain title compound 92 [analytical conditions: Chiral column: CHIRALPAK AS-H, 25×0.46 cm; modifier: (Ethanol+0.1% Isopropylamine) 5% vs CO₂; flow rate=2.5 mL/min; pressure=190 bar; T=35° C.; UV wavelength: 220 nm; retention time=14.8 minutes] (4 mg) and title compound 93 [analytical conditions: Chiral column: CHIRALPAK AS-H, 25×0.46 cm; modifier: (Ethanol+0.1% Isopropylamine) 8% vs CO₂; flow rate=2.5 mL/min; pressure=190 bar; T=35° C.; UV wavelength: 220 nm; retention time=18.2 minutes] (5 mg).

### EXAMPLE 92

MS (ES/+): m/z=458 [M+H]⁺.

NMR (CDCl₃): δ (ppm) 8.16 (s, 1H); 7.91 (td, 1H); 7.87 (td, 1H); 7.6 (d, 1H); 7.59 (d, 1H); 7.46 (d, 1H); 7.24 (dd, 2H); 6.81 (td, 2H); 6.49 (q, 1H); 2.91 (bd, 1H); 2.79 (d, 1H); 2.61 (d, 1H); 2.5 (bt, 1H); 2.6-2.3 (m, 1H); 2.48 (bd, 1H); 2.4 (bt, 1H); 2.35 (s, 3H); 1.94 (tm, 1H); 1.91 (s, 3H); 1.49 (bt, 1H); 1.37 (d, 3H); 1.19 (d, 3H).

### EXAMPLE 93

MS (ES/+): m/z=458 M+H]⁺.

NMR (CDCl₃): δ (ppm) 8.15 (s, 1H); 7.92 (td, 1H); 7.87 (td, 1H); 7.61 (d, 1H); 7.6 (d, 1H); 7.46 (d, 1H); 7.24 (dd, 2H); 6.82 (td, 2H); 6.48 (q, 1H); 2.93 (bd, 1H); 2.78 (d, 1H); 2.62 (d, 1H); 2.59 (bt, 1H); 2.56 (m, 1H); 2.35 (s, 3H); 2.3 (bd, 1H); 1.94 (s, 3H); 1.86 (td, 1H); 1.61 (bd, 1H); 1.33 (d, 3H); 1.3 (m, 1H); 1.17 (d, 3H).

### EXAMPLE 94 AND 95

### N-[1-(3-cyano-1-naphthalenyl)ethyl]-2-[4-(4-fluorophenyl)-1,2-dimethyl-4-piperidinyl]-N-methylacetamide (Syn Isomer 1, Chain Enantiomer 2)

### N-[1-(3-cyano-1-naphthalenyl)ethyl]-2-[4-(4-fluorophenyl)-1,2-dimethyl-4-piperidinyl]-N-methylacetamide (Syn Isomer 2, Chain Enantiomer 2)

Starting from example 91 (55 mg), 49 mg of the mixture of title compounds was obtained as a white foam.

The mixture was then purified by semipreparative SFC (Gilson) chromatography [semipreparative conditions: Chiral column: CHIRALPAK AS-H, 25×2.1 cm; modifier: (Ethanol+0.1% Isopropylamine) 5% vs CO₂; flow rate=22 mL/min; pressure=192 bar; T=36° C.; UV wavelength: 220 nm; loop=1 mL; injection: 10 mg each injection] to obtain title compound 94 [analytical conditions: Chiral column: CHIRALPAK AS-H, 25×0.46 cm; modifier: (Ethanol+0.1% Isopropylamine) 5% vs CO₂; flow rate=2.5 mL/min; pressure=192 bar; T=35° C.; UV wavelength: 220 nm; retention time=14.8 minutes] (12 mg) and title compound 95 [analytical conditions: Chiral column: CHIRALPAK AS-H, 25×0.46 cm; modifier: (Ethanol+0.1% Isopropylamine) 5% vs CO₂; flow rate=2.5 mL/min; pressure 192 bar; T=35° C.; UV wavelength: 220 nm; retention time=16.4 minutes] (5 mg).

### EXAMPLE 94

MS (ES/+): m/z=458 [M+H]⁺.

NMR (CDCl₃): δ (ppm) 8.15 (s, 1H); 7.92 (td, 1H); 7.87 (td, 1H); 7.61 (d, 1H); 7.6 (d, 1H); 7.46 (d, 1H); 7.24 (dd, 2H); 6.82 (td, 2H); 6.48 (q, 1H); 2.95 (bd, 1H); 2.78 (d, 1H); 2.62 (d, 1H); 2.61 (bt, 1H); 2.57 (m, 1H); 2.37 (s, 3H); 2.31 (bd, 1H); 1.93 (s, 3H); 1.86 (td, 1H); 1.62 (bd, 2H); 1.34 (d, 3H); 1.18 (d, 3H).

### EXAMPLE 95

MS (ES/+): m/z=458 [M+H]⁺.

NMR (CDCl₃): δ (ppm) 8.16 (s, 1H); 7.9 (td, 1H); 7.88 (td, 1H); 7.61 (d, 1H); 7.6 (d, 1H); 7.46 (d, 1H); 7.24 (dd, 2H); 6.81 (td, 2H); 6.49 (q, 1H); 2.98 (bd, 1H); 2.77 (d, 1H); 2.61 (d, 1H); 2.57 (bt, 1H); 2.55 (m, 1H); 2.4 (s, 3H); 2.35 (bd, 1H); 2.02 (bm, 1H); 1.89 (s, 3H); 1.61 (bd, 2H); 1.37 (d, 3H); 1.24 (d, 3H).

Following the same procedure described to obtain example 1, examples 96, 97 was prepared.

### EXAMPLE 96

### N-[1-(3-chloro-1-naphthalenyl)ethyl]-2-[4-(4-fluorophenyl)-4-piperidinyl]-N-methylacetamide (Enantiomer 1)

Starting from intermediate 116 (136 mg), 95 mg of the title compound were obtained as a white foam.

HPLC (walk-up) t_{R}=4.78

NMR (CDCl₃): δ (ppm) 7.84 (d, 1H); 7.73 (s, 1H); 7.72 (d, 1H); 7.49 (t, 1H); 7.42 (td, 1H); 7.28 (dd, 2H); 7.27 (d, 1H); 6.86 (t, 2H); 6.47 (q, 1H); 2.97 (bm, 2H); 2.78 (bm, 2H); 2.56 (s, 2H); 2.40 (bm, 1H); 2.27 (bm, 1H); 2.13 (btm, 1H); 2.00 (btm, 1H); 1.92 (s, 3H); 1.34 (d, 3H).

### EXAMPLE 97

### N-[1-(3-chloro-1-naphthalenyl)ethyl]-2-[4-(4-fluorophenyl)-4-piperidinyl]-N-methylacetamide (Enantiomer 2)

Starting from intermediate 117 (171 mg), 120 mg of the title compound were obtained as a white foam.

MS (ES/+): m/z=439 [M+H]⁺.

NMR (CDCl₃): δ (ppm) 7.84 (d, 1H); 7.73 (s, 1H); 7.72 (d, 1H); 7.49 (t, 1H); 7.42 (td, 1H); 7.28 (dd, 2H); 7.27 (d, 1H); 6.86 (t, 2H); 6.47 (q, 1H); 2.97 (bm, 2H); 2.78 (bm, 2H); 2.56 (s, 2H); 2.40 (bm, 1H); 2.27 (bm, 1H); 2.13 (btm, 1H); 2.00 (btm, 1H); 1.92 (s, 3H); 1.34 (d, 3H).

Following the same procedure described to obtain example 2, examples 98, and 100 was prepared.

### EXAMPLE 98

### N-[1-(3-chloro-1-naphthalenyl)ethyl]-2-[4-(4-fluorophenyl)-1-methyl-4-piperidinyl]-N-methylacetamide (Enantiomer 1)

Starting from example 96 (30 mg), 30 mg of the title compound were obtained as a white solid without any chromatographic purification.

MS (ES/+): m/z=453 [M+H]⁺.

NMR (CDCl₃): δ (ppm) 7.81 (d, 1H); 7.73 (s, 1H); 7.72 (d, 1H); 7.48 (t, 1H); 7.41 (t, 1H); 7.28 (s, 1H); 7.27 (dd, 2H); 6.86 (t, 2H); 6.45 (q, 1H); 2.62 (bm, 2H); 2.55 (s, 2H); 2.6-2.3 (bm, 2H); 2.40-2.0 (bm, 4H); 2.23 (s, 3H); 1.91 (s, 3H); 1.32 (d, 3H).

Following the same procedure described to obtain example 3, example 99 was prepared.

### EXAMPLE 99

### N-[1-(3-chloro-1-naphthalenyl)ethyl]-2-[4-(4-fluorophenyl)-1-methyl-4-piperidinyl]-N-methylacetamide Hydrochloride (Enantiomer 1)

Starting from example 98 (27 mg), 21 mg of the title compound were obtained as a white solid.

MS (ES/+): m/z=453 [M+H]⁺.

NMR (d₆ DMSO): δ (ppm) 10.2 (bs, 1H); 8.05 (d, 1H); 7.98 (d, 1H); 7.75 (bt, 1H); 7.61 (t, 1H); 7.49 (d, 1H); 7.46 (bm, 2H); 7.41 (bm, 1H); 7.06 (bm, 2H); 6.32 (m, 1H); 3.4 (m, 2H); 2.8 (m, 2H); 2.8-2.6 (bm, 2H); 2.6-2.0 (m, 4H); 2.71 (bs, 3H); 2.09 (bs, 3H); 1.35 (d, 3H).

### EXAMPLE 100

### N-[1-(3-chloro-1-naphthalenyl)ethyl]-2-[4-(4-fluorophenyl)-1-methyl-4-piperidinyl]-N-methylacetamide (Enantiomer 2)

Starting from example 97 (30 mg), 30 mg of the title compound were obtained as a white solid without any chromatographic purification.

MS (ES/+): m/z=453 [M+H]⁺.

NMR (CDCl₃): δ (ppm) 7.81 (d, 1H); 7.73 (s, 1H); 7.72 (d, 1H); 7.48 (t, 1H); 7.41 (t, 1H); 7.28 (s, 1H); 7.27 (dd, 2H); 6.86 (t, 2H); 6.45 (q, 1H); 2.62 (bm, 2H); 2.55 (s, 2H); 2.6-2.3 (bm, 2H); 2.40-2.0 (bm, 4H); 2.23 (s, 3H); 1.91 (s, 3H); 1.32 (d, 3H).

Following the same procedure described to obtain example 1, examples 101, 102 were prepared.

### EXAMPLE 101

### N-[1-(3-chloro-1-naphthalenyl)ethyl]-N-methyl-2-(2-methyl-4-phenyl-4-Piperidinyl)acetamide (Syn Isomer 1, Chain Enantiomer 1)

Starting from intermediate 131 (50 mg), 33 mg of the title compound were obtained as a white foam.

HPLC (walk-up): t_{R}=4.76

NMR (CDCl₃): δ (ppm) 7.82 (d, 1H); 7.72 (s, 1H); 7.71 (d, 1H); 7.49 (td, 1H); 7.45 (td, 1H); 7.22-7.31 (m, 3H); 7.16 (d, 1H); 7.15 (d, 2H); 6.45 (q, 1H); 3.36 (bd, 1H); 3.17 (bt, 1H); 2.82 (bd, 1H); 2.8 (d, 1H); 2.57 (d, 1H); 2.39 (bd, 1H); 2.03 (td, 1H); 1.76 (s, 3H); 1.59 (bt, 1H); 1.43 (d, 3H); 1.4 (bd, 1H); 1.31 (d, 3H).

### EXAMPLE 102

### N-[1-(3-chloro-1-naphthalenyl)ethyl]-N-methyl-2-(2-methyl-4-phenyl-4-piperidinyl)acetamide (Syn Isomer 2, Chain Enantiomer 1)

Starting from intermediate 132 (34 mg), 28 mg of the title compound were obtained as a white foam.

MS (ES/+): m/z=436 [M+H]⁺.

NMR (CDCl₃): δ (ppm) 7.82 (d, 1H); 7.72 (s, 1H); 7.71 (d, 1H); 7.49 (td, 1H); 7.45 (td, 1H); 7.22-7.31 (m, 3H); 7.16 (d, 1H); 7.15 (d, 2H); 6.45 (q, 1H); 3.36 (bd, 1H); 3.17 (bt, 1H); 2.82 (bd, 1H); 2.8 (d, 1H); 2.57 (d, 1H); 2.39 (bd, 1H); 2.03 (td, 1H); 1.76 (s, 3H); 1.59 (bt, 1H); 1.43 (d, 3H); 1.4 (bd, 1H); 1.31 (d, 3H).

Following the same procedure described to obtain example 2, examples 103, 104 were prepared.

### EXAMPLE 103

### N-[1-(3-chloro-1-naphthalenyl)ethyl]-2-(1,2-dimethyl-4-phenyl-4-piperidinyl)-N-methylacetamide (Syn Isomer 1, Chain Enantiomer 1)

Starting from example 101 (24 mg), 18 mg of the title compound were obtained as a white foam.

MS (ES/+): m/z=449 [M+H]⁺.

NMR (CDCl₃): δ (ppm) 7.89 (d, 1H); 7.78 (s, 1H); 7.76 (d, 1H); 7.55 (td, 1H); 7.5 (td, 1H); 7.34-7.22 (m, 3H); 7.17 (d, 1H); 7.16 (d, 2H); 6.52 (q, 1H); 3.15 (bm, 1H); 2.74 (bd, 2H); 2.8 (d, 1H); 2.67 (d, 1H); 2.51 (bs, 3H); 2.4 (dm, 1H); 2.2 (td, 1H); 1.78 (s, 3H); 1.81 (tm, 2H); 1.37 (d, 3H); 1.35 (bm, 3H).

[α]_{D}=−119.8 (c=0.54, CHCl₃)

### EXAMPLE 104

### N-[1-(3-chloro-1-naphthalenyl)ethyl]-2-(1,2-dimethyl-4-phenyl-4-piperidinyl)-N-methylacetamide (Syn Isomer 2, Chain Enantiomer 1)

Starting from example 102 (24 mg), 20 mg of the title compound were obtained as a white foam.

MS (ES/+): m/z=449 [M+H]⁺.

NMR (CDCl₃): δ (ppm) 7.87 (d, 1H); 7.75 (s, 1H); 7.71 (d, 1H); 7.5 (td, 1H); 7.48 (td, 1H); 7.34-7.22 (m, 3H); 7.15 (d, 1H); 7.14 (d, 2H); 6.5 (q, 1H); 3.25 (bm, 1H); 2.74 (bm, 2H); 2.8 (d, 1H); 2.67 (d, 1H); 2.61 (bs, 3H); 2.4 (dm, 1H); 2.3 (td, 1H); 1.75 (s, 3H); 1.81 (tm, 2H); 1.4 (bm, 3H); 1.36 (d, 3H).

[α]_{D}=−103.9 (c=0.37, CHCl₃)

Following the same procedure described to obtain example 1, examples 105, 106 was prepared.

### EXAMPLE 105

### N-[1-(3-chloro-1-naphthalenyl)ethyl]-N-methyl-2-(2-methyl-4-phenyl-4-piperidinyl)acetamide (Syn Isomer 1, Chain Enantiomer 2)

Starting from intermediate 133 (38 mg), 28 mg of the title compound were obtained as a white foam.

MS (ES/+): m/z=436 [M+H]⁺.

NMR (CDCl₃): δ (ppm) 7.89 (d, 1H); 7.78 (s, 1H); 7.77 (d, 1H); 7.55 (td, 1H); 7.5 (td, 1H); 7.34-7.22 (m, 3H); 7.18 (d, 1H); 7.16 (d, 2H); 6.5 (q, 1H); 3.47 (bm, 1H); 3.39 (m, 1H); 2.84 (bd, 1H); 2.8 (d, 1H); 2.67 (d, 1H); 2.55 (dm, 1H); 2.03 (td, 1H); 1.86 (s, 3H); 1.81 (tm, 2H); 1.51 (d, 3H); 1.35 (d, 3H).

### EXAMPLE 106

### N-[1-(3-chloro-1-naphthalenyl)ethyl]-N-methyl-2-(2-methyl-4-phenyl-4-piperidinyl)acetamide (Syn Isomer 2, Chain Enantiomer 2)

Starting from intermediate 134 (42 mg), 25 mg of the title compound were obtained as a white foam.

MS (ES/+): m/z=436 [M+H]⁺.

NMR (CDCl₃): δ (ppm) 7.82 (d, 1H); 7.72 (s, 1H); 7.71 (d, 1H); 7.49 (td, 1H); 7.45 (td, 1H); 7.22-7.31 (m, 3H); 7.16 (d, 1H); 7.15 (d, 2H); 6.45 (q, 1H); 3.36 (bd, 1H); 3.17 (bt, 1H); 2.82 (bd, 1H); 2.8 (d, 1H); 2.57 (d, 1H); 2.39 (bd, 1H); 2.03 (td, 1H); 1.76 (s, 3H); 1.59 (bt, 1H); 1.43 (d, 3H); 1.4 (bd, 1H); 1.31 (d, 3H).

Following the same procedure described to obtain example 2, examples 107, 108 were prepared.

### EXAMPLE 107

### N-[1-(3-chloro-1-naphthalenyl)ethyl]-2-(1,2-dimethyl-4-phenyl-4-piperidinyl)-N-methylacetamide (Syn Isomer 1, Chain Enantiomer 2)

Starting from example 105 (24 mg), 22 mg of the title compound were obtained as a white foam.

MS (ES/+): m/z=449 [M+H]⁺.

NMR (CDCl₃): δ (ppm) 7.93 (d, 1H); 7.78 (s, 1H); 7.77 (d, 1H); 7.55 (td, 1H); 7.5 (td, 1H); 7.34-7.22 (m, 3H); 7.15 (d, 1H); 7.14 (d, 2H); 6.52 (q, 1H); 3.25 (bm, 1H); 3.0 (bm, 1H); 2.74 (bd, 1H); 2.8 (d, 1H); 2.67 (d, 1H); 2.61 (bs, 3H); 2.4 (dm, 1H); 2.2 (td, 1H); 1.79 (s, 3H); 1.81 (tm, 2H); 1.4 (bm, 3H); 1.36 (d, 3H).

[α]_{D}=+114.4 (c=0.86, CHCl₃)

### EXAMPLE 108

### N-[1-(3-chloro-1-naphthalenyl)ethyl]-2-(1,2-dimethyl-4-phenyl-4-piperidinyl)-N-methylacetamide (Syn Isomer 2, Chain Enantiomer 2)

Starting from example 106 (24 mg), 20 mg of the title compound were obtained as a white foam.

MS (ES/+): m/z=449 [M+H]⁺.

NMR (CDCl₃): δ (ppm) 7.89 (d, 1H); 7.78 (s, 1H); 7.76 (d, 1H); 7.55 (td, 1H); 7.5 (td, 1H); 7.34-7.22 (m, 3H); 7.17 (d, 1H); 7.16 (d, 2H); 6.52 (q, 1H); 3.15 (bm, 1H); 2.74 (bd, 2H); 2.8 (d, 1H); 2.67 (d, 1H); 2.51 (bs, 3H); 2.4 (dm, 1H); 2.2 (td, 1H); 1.78 (s, 3H); 1.81 (tm, 2H); 1.37 (d, 3H); 1.35 (bm, 3H).

[α]_{D}=+102.3 (c=0.86, CHCl₃).

### EXAMPLE 109

### N-[1-(3-chloro-1-naphthalenyl)ethyl]-2-[4-(4-fluorophenyl)-2-methyl-4-piperidinyl]-N-methylacetamide (Syn Isomer 1, Chain Enantiomer 1)

Intermediate 103 was dissolved in dry DMF (2 mL) and, under a Nitrogen atmosphere and at 0° C., NaH 60% dispersion in mineral oil (20 mg) was added. The mixture was allowed to warm to rt and stirred under these conditions for 20 min. Then methyl iodide was added (0.064 mL) and the solution was stirred overnight at rt. Water and AcOEt were added; the organic phase separated, dried and evaporated under vacuum to give a compound intermediate without any further purification [T.I.c. CH:AcOEt=7:3 Rf=0.29]. TFA (0.5 mL) was added to a solution of this intermediate (103 mg) in anhydrous DCM (2 mL) at 0° C. under a Nitrogen atmosphere. The mixture was stirred 1 h, then aqueous 2M NaOH was added up to basic pH and the resulting solution filtered through a phase separation cartridge with polypropylene frit and concentrated under vacuum. The residue was purified by flash chromatography eluting with DCM 100% to DCM MeOH 7:3 to afford the title compound (56 mg) as a white foam.

MS (ES/+): m/z=453 [M+H]⁺.

NMR (CDCl₃): δ (ppm) 7.81 (d, 1H); 7.74 (s, 1H); 7.73 (d, 1H); 7.5 (td, 1H); 7.43 (td, 1H); 7.28 (d, 1H); 7.22 (dd, 2H); 6.82 (td, 2H); 6.45 (q, 1H); 3.33 (m, 1H); 3.25 (bd, 1H); 2.76 (d, 1H); 2.7 (bt, 1H); 2.63 (d, 1H); 2.48 (bd, 1H); 1.97 (bd, 1H); 1.92 (s, 3H); 1.87 (bt, 1H); 1.63 (bt, 1H); 1.38 (d, 3H); 1.33 (d, 3H).

Following the same procedure described to obtain example 109, examples 110, 111, 112 were prepared.

### EXAMPLE 110

### N-[1-(3-chloro-1-naphthalenyl)ethyl]-2-[4-(4-fluorophenyl)-2-methyl-4-piperidinyl]-N-methylacetamide (Syn Isomer 2, Chain Enantiomer 1)

Starting from intermediate 104 (65 mg), 24 mg of the title compound were obtained as a white foam.

MS (ES/+): m/z=453 [M+H]⁺.

NMR (CDCl₃): δ (ppm) 7.79 (d, 1H); 7.74 (s, 1H); 7.73 (d, 1H); 7.5 (td, 1H); 7.43 (td, 1H); 7.28 (d, 1H); 7.24 (dd, 2H); 6.84 (td, 2H); 6.45 (q, 1H); 3.38 (m, 1H); 3.34 (bd, 1H); 3.16 (bt, 1H); 2.78 (d, 1H); 2.74 (bd, 1H); 2.6 (d, 1H); 2.43 (bd, 1H); 1.96 (bt, 1H); 1.88 (s, 3H); 1.54 (bt, 1H); 1.4 (d, 3H); 1.34 (d, 3H).

### EXAMPLE 111

### N-[1-(3-chloro-1-naphthalenyl)ethyl]-2-[4-(4-fluorophenyl)-2-methyl-4-piperidinyl]-N-methylacetamide (Syn Isomer 1, Chain Enantiomer 2)

Starting from intermediate 105 (100 mg), 59 mg of the title compound were obtained as a white foam.

MS (ES/+): m/z=453 [M+H]⁺.

NMR (CDCl₃): δ (ppm) 7.81 (d, 1H); 7.75 (s, 1H); 7.73 (d, 1H); 7.5 (td, 1H); 7.43 (td, 1H); 7.28 (d, 1H); 7.24 (dd, 2H); 6.82 (td, 2H); 6.45 (q, 1H); 3.25 (m, 1H); 3.25 (bd, 1H); 2.77 (d, 1H); 2.64 (bt, 1H); 2.63 (d, 1H); 2.44 (bd, 1H); 1.92 (s, 3H); 1.81 (td, 1H); 1.56 (bt, 1H); 1.32 (d, 3H); 1.32 (d, 3H); 1.28 (bt, 1H).

### EXAMPLE 112

### N-[1-(3-chloro-1-naphthalenyl)ethyl]-2-[4-(4-fluorophenyl)-2-methyl-4-piperidinyl]-N-methylacetamide (Syn Isomer 2, Chain Enantiomer 2)

Starting from intermediate 106 (87 mg), 50 mg of the title compound were obtained as a white foam.

MS (ES/+): m/z=453 [M+H]⁺.

NMR (CDCl₃): δ (ppm) 7.8 (d, 1H); 7.74 (s, 1H); 7.73 (d, 1H); 7.5 (td, 1H); 7.43 (td, 1H); 7.28 (d, 1H); 7.22 (dd, 2H); 6.83 (td, 2H); 6.45 (q, 1H); 3.33 (m, 1H); 3.25 (bd, 1H); 2.79 (d, 1H); 2.7 (bt, 1H); 2.59 (d, 1H); 2.39 (bd, 1H); 1.9 (bd, 2H); 1.88 (s, 3H); 1.42 (bt, 1H); 1.34 (d, 3H); 1.32 (d, 3H).

Following the same procedure described to obtain example 2, examples 113, 114, 115, 116 were prepared.

### EXAMPLE 113

### N-[1-(3-chloro-1-naphthalenyl)ethyl]-2-[4-(4-fluorophenyl)-1,2-dimethyl-4-piperidinyl]-N-methylacetamide (Syn Isomer 1, Chain Enantiomer 1)

Starting from example 109 (43 mg), 44 mg of the title compound were obtained as a white solid.

MS (ES/+): m/z=467 [M+H]⁺.

NMR (CDCl₃): δ (ppm) 7.87 (d, 1H); 7.77 (s, 1H); 7.76 (d, 1H); 7.53 (td, 1H); 7.47 (td, 1H); 7.3 (d, 1H); 7.22 (dd, 2H); 6.8 (td, 2H); 6.49 (q, 1H); 3.24 (bd, 1H); 2.77 (d, 1H); 2.68 (bd, 1H); 2.64 (d, 1H); 2.61 (bm, 1H); 2.6 (bm, 2H); 2.6 (bs, 3H); 2.41 (bd, 1H); 1.87 (s, 3H); 1.84 (bm, 1H); 1.57 (bm, 1H); 1.37 (d, 3H); 1.37 (d, 3H).

[α]_{D}=−140.4 (c=0.955, CHCl₃)

### EXAMPLE 114

### N-[1-(3-chloro-1-naphthalenyl)ethyl]-2-[4-(4-fluorophenyl)-1,2-dimethyl-4-piperidinyl]-N-methylacetamide (Syn Isomer 2, Chain Enantiomer 1)

Starting from example 110 (19 mg), 19 mg of the title compound were obtained as a white solid.

MS (ES/+): m/z=467 [M+H]⁺.

NMR (CDCl₃): δ (ppm) 7.82 (d, 1H); 7.76 (s, 1H); 7.75 (d, 1H); 7.52 (td, 1H); 7.46 (td, 1H); 7.3 (d, 1H); 7.24 (dd, 2H); 6.83 (td, 2H); 6.47 (q, 1H); 3.15 (bd, 1H); 2.81 (bm, 1H); 2.79 (d, 1H); 2.69 (bt, 1H); 2.61 (d, 1H); 2.57 (bd, 2H); 2.55 (bs, 3H); 2.41 (bd, 1H); 1.86 (s, 3H); 1.84 (bm, 1H); 1.62 (bm, 1H); 1.38 (d, 3H); 1.38 (d, 3H).

[α]_{D}=−91.4 (c=0.507, CHCl₃)

### EXAMPLE 115

### N-[1-(3-chloro-1-naphthalenyl)ethyl]-2-[4-(4-fluorophenyl)-1,2-dimethyl-4-piperidinyl]-N-methylacetamide (Syn Isomer 1, Chain Enantiomer 2)

Starting from example 111 (47 mg), 47 mg of the title compound were obtained as a white solid.

MS (ES/+): m/z=467 [M+H]⁺.

NMR (CDCl₃): δ (ppm) 7.84 (d, 1H); 7.75 (s, 1H); 7.74 (d, 1H); 7.5 (td, 1H); 7.44 (td, 1H); 7.28 (d, 1H); 7.2 (dd, 2H); 6.79 (td, 2H); 6.46 (q, 1H); 3.15 (bd, 1H); 2.76 (d, 1H); 2.65 (bd, 1H); 2.61 (d, 1H); 2.59 (bm, 1H); 2.52 (s, 3H); 2.36 (bd, 1H); 2.12 (bm, 1H); 1.86 (s, 3H); 1.84 (bm, 1H); 1.6 (bm, 1H); 1.34 (d, 3H); 1.34 (d, 3H).

[α]_{D}=+134.6 (c=0.935, CHCl₃)

### EXAMPLE 116

### N-[1-(3-chloro-1-naphthalenyl)ethyl]-2-[4-(4-fluorophenyl)-1,2-dimethyl-4-piperidinyl]-N-methylacetamide (Syn Isomer 2, Chain Enantiomer 2)

Starting from example 112 (38 mg), 39 mg of the title compound were obtained as a white solid.

MS (ES/+): m/z=467 [M+H]⁺.

NMR (CDCl₃): δ (ppm) 7.81 (d, 1H); 7.75 (s, 1H); 7.73 (d, 1H); 7.5 (td, 1H); 7.44 (td, 1H); 7.28 (d, 1H); 7.24 (dd, 2H); 6.82 (td, 2H); 6.46 (q, 1H); 3 (bd, 1H); 2.79 (d, 1H); 2.63 (bm, 1H); 2.59 (d, 1H); 2.57 (bt, 1H); 2.42 (s, 3H); 2.37 (bd, 2H); 2.07 (bt, 1H); 1.86 (s, 3H); 1.6 (bt, 1H); 1.34 (d, 3H); 1.25 (d, 3H).

[α]_{D}=+91.2 (c=1.135, CHCl₃)

### Pharmacy Examples

### A. Capsules/Tablets

| | | |
|---|---|---|
| Active ingredient | 25.0 | mg |
| PVP | 2.5 | mg |
| Microcrystalline Cellulose | 198.5 | mg |
| Croscarmellose Sodium | 2.5 | mg |
| Magnesium Stearate | 1.5 | mg |

The active ingredient is blended with the other excipients. The blend can be used to fill gelatin capsules or compressed to form tablets using appropriate punches. The tablets can be coated using conventional techniques and coatings.

### B. Tablets

| | | |
|---|---|---|
| Active ingredient | 25.0 | mg |
| Microcrystalline Cellulose | 264.0 | mg |
| Croscarmellose Sodium | 10.0 | mg |
| Magnesium Stearate | 1.0 | mg |

The active ingredient is blended with microcrystalline cellulose and croscarmellose sodium. Magnesium stearate is then added to the previous blend. The mixture thus obtained can be compressed using appropriate punches and the tablets coated using conventional techniques and coatings.

### C) Infusion

| | |
|---|---|
| Active ingredient | 2-50 mg/ml |
| Buffer solution pH 4.5 suitable for infusion | qs to 100 ml |
| (e.g. sodium citrate in NaCl 0.9% or 5% dextrose) | |

The formulation may be packed in glass vials or plastic bag.

### Biology Data

The affinity of the compound of the invention for the NK₁ receptor was determined using the NK₁ receptor binding affinity method measuring in vitro by the compounds' ability to displace [3H]—substance P (SP) from recombinant human NK₁ receptors expressed in Chinese Hamster Ovary (CHO) cell membranes. The affinity values are expressed as negative logarithm of the inhibition constant (Ki) of displacer ligands (pKi).

The pKi values obtained as the average of at least two determinations with representative compounds of the invention are within the range of 9.82 to 6.52.

The affinity of the compound of the invention for the serotonin transporter was determined using the hSERT binding affinity method and measuring in vitro the compounds' ability to displace [³H]—citalopram from recombinant human serotonin transporter expressed in Porcine Epithelial Kidney LLCPK cell membranes. The affinity values are expressed as negative logarithm of the inhibition constant (Ki) of displacer ligands (pKi).

The pKi values obtained as the average of at least two determinations with representative compounds of the invention are within the range of 9.71 to 6.54.

1. A compound of formula (I) 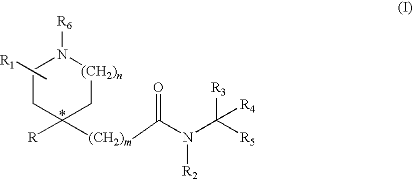
wherein R is a radical selected from 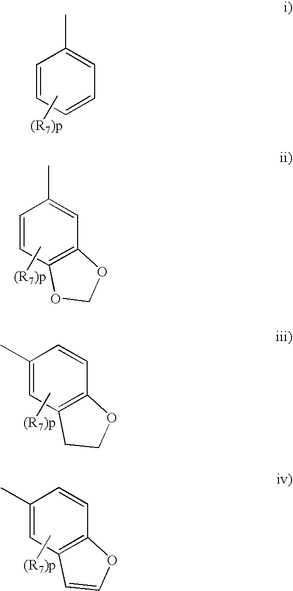
in which R₇ is halogen, cyano, C₁₋₄ alkyl, C₁₋₄ alkoxy, trifluoromethyl or trifluoromethoxy;
p is an integer from 0 to 3;
R₁ is hydrogen, halogen, cyano, C₂₋₄ alkenyl, C₁₋₄ alkyl optionally substituted by halogen, cyano or C₁₋₄ alkoxy;
R₂ is hydrogen or C₁₋₄ alkyl;
R₃ and R₄ independently are hydrogen, C₁₋₄ alkyl or R₃ together with
R₄ is C₃₋₇ cycloalkyl;
R₅ is:
phenyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl,
naphthyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl,
a 9 to 10 membered fused bicyclic heterocyclic group substituted by 1 to 3 groups independently selected from trifluoromethyl, C₁₋₄ alkyl, cyano, C₁₋₄ alkoxy, trifluoromethoxy, halogen or (SO)rC₁₋₄ alkyl or
R₅ is a 5 or 6 membered heteroaryl group substituted by 1 to 3 groups independently selected from trifluoromethyl, C₁₋₄ alkyl, cyano, C₁₋₄ alkoxy, trifluoromethoxy, halogen or (SO)rC₁₋₄ alkyl;
R₆ is hydrogen or (CH₂)qR₈;
R₈ is hydrogen, C₃₋₇ cycloalkyl, C₁₋₄ alkoxy, amine, C₁₋₄ alkylamine, (C₁₋₄ alkyl)₂amine, OC(O)NR₉R₁₀ or C(O)NR₉R₁₀;
R₉ and R₁₀ independently are hydrogen, C₁₋₄ alkyl or C₃₋₇ cycloalkyl;
m is zero or 1;
n is 1 or 2;
q is an integer from 1 to 4;
r is 1 or 2;
provided that when R₅ is phenyl substituted by 1 to 3 groups independently selected from trifluoromethyl, C₁₋₄ alkyl, cyano, C₁₋₄ alkoxy, trifluoromethoxy, halogen or (SO)rC₁₋₄ alkyl, R is not the radical i) 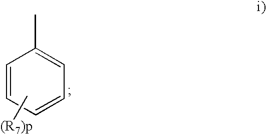
or a pharmaceutically acceptable salt or solvate thereof.

2. A compound as claimed in claim 1 wherein m is 1.

3. A compound as claimed in claim 1 wherein n is 1.

4. A compound as claimed in claim 1 wherein R₆ is hydrogen or C₁₋₄ alkyl.

5. A compound as claimed in claim 1 wherein R₁ is hydrogen, C₂₋₄ alkenyl, halogen or C₁₋₄ alkyl.

6. A compound as claimed in claim 1 wherein R₂, R₃ and R₄ are independently hydrogen or methyl.

7. A compound as claimed in claim 1 wherein R₅ is phenyl substituted by one or two groups selected from fluorine, bromine, chlorine, cyano, or methyl, naphthyl substituted by one or two groups selected from fluorine, bromine, chlorine, cyano, or methyl, benzofuranyl substituted by one or two groups selected from fluorine, bromine, chlorine, cyano, or methyl, or R₅ is furanyl substituted by one or two groups selected from fluorine, bromine, chlorine, cyano, or methyl.

8. A compound as claimed in claim 1 wherein R is phenyl in which R₇ is halogen, cyano, C₁₋₄ alkyloxy, trifluoromethyl or C₁₋₄ alkyl and within this class p is 0 or an integer from 1 to 2 or R is a group selected from 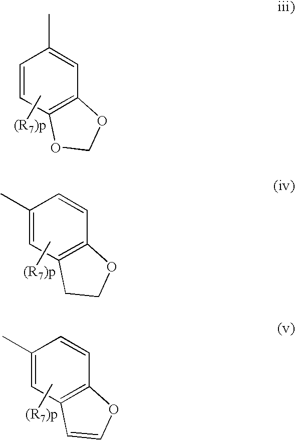
wherein p is 0.

9. A compound as claimed in claim 1 wherein n and m are 1, R₂ is hydrogen or methyl, R₃ is hydrogen, R₄ is hydrogen or methyl, R₅ is phenyl substituted by one or two groups selected from fluorine, bromine or chlorine, cyano, or methyl, 1-naphthyl substituted by one or two groups selected from fluorine, bromine or chlorine, cyano, or methyl, or R₅ is benzofuran-7-yl substituted by a fluorine, bromine or chlorine, cyano, or methyl, R₆ is hydrogen or methyl, R₁ is hydrogen, ethenyl, fluorine or methyl at the 1 or 2 position of the piperidine ring and R is phenyl in which R₇ is fluorine, methoxy, cyano or methyl and p is 0 or an integer from 1 to 2 or R is a group selected from 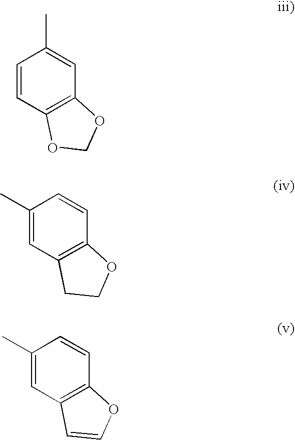
wherein p is 0.

10. A compound selected from:
N-[1-(3-chloro-1-naphthalenyl)ethyl]-2-[4-(4-fluorophenyl)-1-methyl-4-piperidinyl]-N-methylacetamide (Enantiomer 1);
N-[1-(3-chloro-1-naphthalenyl)ethyl]-N-methyl-2-(1-methyl-4-phenyl-4-piperidinyl)acetamide (Enantiomer 1);
N-[1-(3-chloro-1-naphthalenyl)ethyl]-N-methyl-2-(1-methyl-4-phenyl-4-piperidinyl)acetamide (Enantiomer 2);
2-[4-(1-benzofuran-5-yl)-1-methyl-4-piperidinyl]-N-[1-(3-chloro-1-naphthalenyl)ethyl]-N-methylacetamide (Enantiomer 1);
N-[1-(3-chloro-1-naphthalenyl)ethyl]-N-methyl-2-{1-methyl-4-[4-(methyloxy)phenyl]-4-piperidinyl}acetamide (Enantiomer 1);
N-[1-(3-chloro-1-naphthalenyl)ethyl]-2-[4-(4-fluorophenyl)-1,2-dimethyl-4-piperidinyl]-N-methylacetamide (Syn isomer 2, chain enantiomer 1);
N-[1-(3-chloro-1-naphthalenyl)ethyl]-2-(1,2-dimethyl-4-phenyl-4-piperidinyl)-N-methylacetamide (Syn isomer 2, chain enantiomer 1);
and pharmaceutically acceptable salts or solvates thereof.

11. A process (A) for the preparation of a compound as claimed in claim 1 which comprises reacting an activated derivative of the carboxylic acid of formula (II) wherein R₆ is a nitrogen protecting group or (CH₂)_{q}R₈, with amine (III) 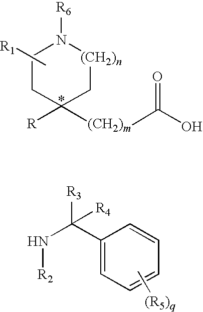
wherein R₂ is hydrogen, C₁₋₄ alkyl or a nitrogen protecting group, followed where necessary by removal of any nitrogen protecting group; or
a process B for the preparation of a compound of formula (I) wherein R₂ is C₁₋₄ alkyl which comprises the reaction of a compound of formula (I), wherein R₂ is hydrogen, with (C₁₋₄ alkyl)L, wherein L is a suitable leaving group selected from iodine, bromine, in the presence of a base.

12-14. (canceled)

15. A pharmaceutical composition comprising a compound as claimed in claim 1 in admixture with one or more pharmaceutically acceptable carriers or excipients.

16. (canceled)

17. N-[1-(3-chloro-1-naphthalenyl)ethyl]-2-[4-(4-fluorophenyl)-1-methyl-4-piperidinyl]-N-methylacetamide or a pharmaceutically acceptable salt thereof.

18. An enantiomer of the compound as claimed in claim 17.

19. The compound as claimed in claim 17, which is N-[1-(3-chloro-1-naphthalenyl)ethyl]-2-[4-(4-fluorophenyl)-1-methyl-4-piperidinyl]-N-methylacetamide hydrochloride.

20. An enantiomer of the compound as claimed in claim 19.

21. The pharmaceutical composition as claimed in claim 15 wherein the compound is N-[1-(3-chloro-1-naphthalenyl)ethyl]-2-[4-(4-fluorophenyl)-1-methyl-4-piperidinyl]-N-methylacetamide or a pharmaceutically acceptable salt thereof.

22. The pharmaceutical composition as claimed in claim 15, wherein the compound is N-[1-(3-chloro-1-naphthalenyl)ethyl]-2-[4-(4-fluorophenyl)-1-methyl-4-piperidinyl]-N-methylacetamide hydrochloride.

23. The pharmaceutical composition as claimed in claim 15 wherein the compound is an enantiomer of N-[1-(3-chloro-1-naphthalenyl)ethyl]-2-[4-(4-fluorophenyl)-1-methyl-4-piperidinyl]-N-methylacetamide or a pharmaceutically acceptable salt thereof.

24. A method for the treatment of inflammation in rheumatoid arthritis in a mammal in need thereof, comprising administering an effective amount of a compound as claimed in claim 1.

25. The method according to claim 24, wherein the compound is N-[1-(3-chloro-1-naphthalenyl)ethyl]-2-[4-(4-fluorophenyl)-1-methyl-4-piperidinyl]-N-methylacetamide or a pharmaceutically acceptable salt thereof.

26. The method according to claim 24, wherein the compound is an enantiomer of N-[1-(3-chloro-1-naphthalenyl)ethyl]-2-[4-(4-fluorophenyl)-1-methyl-4-piperidinyl]-N-methylacetamide or a pharmaceutically acceptable salt thereof.

27. A method for the treatment of a depressive state in a mammal in need thereof, comprising administering a compound as claimed in claim 1.

28. The method as claimed in claim 27, wherein said depressive state is Major Depressive Disorder.

29. The method as claimed in claim 27, wherein the compound is N-[1-(3-chloro-1-naphthalenyl)ethyl]-2-[4-(4-fluorophenyl)-1-methyl-4-piperidinyl]-N-methylacetamide or a pharmaceutically acceptable salt thereof.

30. The method as claimed in claim 27, wherein the compound is an enantiomer of N-[1-(3-chloro-1-naphthalenyl)ethyl]-2-[4-(4-fluorophenyl)-1-methyl-4-piperidinyl]-N-methylacetamide or a pharmaceutically acceptable salt thereof.

31. A method for the treatment of anxiety in a mammal in need thereof, comprising administering a compound as claimed in claim 1.

32. The method as claimed in claim 31, wherein the compound is N-[1-(3-chloro-1-naphthalenyl)ethyl]-2-[4-(4-fluorophenyl)-1-methyl-4-piperidinyl]-N-methylacetamide or a pharmaceutically acceptable salt thereof.

33. The method as claimed in claim 31, wherein the compound is an enantiomer of N-[1-(3-chloro-1-naphthalenyl)ethyl]-2-[4-(4-fluorophenyl)-1-methyl-4-piperidinyl]-N-methylacetamide or a pharmaceutically acceptable salt thereof.

34. A method for the treatment of emesis in a mammal in need thereof, comprising administering a compound as claimed in claim 1.

35. The method as claimed in claim 34, wherein the compound is N-[1-(3-chloro-1-naphthalenyl)ethyl]-2-[4-(4-fluorophenyl)-1-methyl-4-piperidinyl]-N-methylacetamide or a pharmaceutically acceptable salt thereof.

36. The method as claimed in claim 34, wherein the compound is an enantiomer of N-[1-(3-chloro-1-naphthalenyl)ethyl]-2-[4-(4-fluorophenyl)-1-methyl-4-piperidinyl]-N-methylacetamide or a pharmaceutically acceptable salt thereof.
